(19) United States
(12) Patent Application Publication
(10) Pub. No.: US 2010/0267717 A1
(43) Pub. Date: Oct. 21, 2010

(54) Novel Heterocyclic NF-kB Inhibitors

(75) Inventors: Johann Leban, Germering (DE); Harald Schmitt, Mainz (DE); Kristina Wolf, Muenchen (DE); Stefano Pegoraro, Planegg (DE); Andreas Wuzik, Untermeitingen (DE)

Correspondence Address: Baker Donelson Bearman Caldwell & Berkowitz PC;920 Massachusetts Avenue, Suite 900, Washington, DC, 20001, US

(73) Assignee: 4SC AG, Martinsried (DE)

(21) Appl. No.: 11/686,301

(22) Filed: Mar. 14, 2007

Related U.S. Application Data
(63) Continuation-in-part of application No. 11/375,259, filed on Mar. 15, 2006, now Pat. No. 7,601,745.
Application No. 11/375,259 is a continuation-in-part of application No. 11/192,009, filed on Jul. 29, 2005.

(51) Int. Cl. A61K 31/5377; C07D 401/14; A61K 31/497; A61K 31/496; C07D 487/04; A61K 31/53; C07D 417/14; A61P 9/10; A61P 25/16; A61P 25/28; A61P 25/08; A61P 17/06; A61P 17/00; A61P 17/02; A61P 31/14; A61P 31/18; A61P 31/16; A61P 19/10

(52) U.S. Cl. 514/234.5; 544/405; 514/255.05; 544/364; 514/253.1; 544/184; 514/243; 544/133; 544/124

(57) ABSTRACT

The present invention relates in one embodiment to compounds of formula (Ih) and/or a pharmaceutically acceptable salt thereof with an acid or a base, and/or a pharmaceutically acceptable prodrug and/or a stereoisomer thereof,

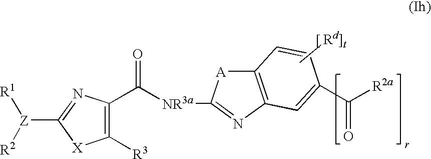

wherein
A is NR^{2′}, S or O;
R^{3a} is H, OH, SH, NH₂, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, alkyl, cycloalkyl, hydroxyalkyl, haloalkyl, haloalkyloxy, alkoxy, alkylamino, hydroxyalkylamino, halogen, aryl, or heteroaryl;
R³ is H, —C(O)NR^{a}R^{b}, halogen, alkyl, haloalkyl, aryl, heteroaryl, OH, SH, NR^{4′}OR^{5′}, NH₂, hydroxyalkylamino, alkylamino, alkoxy, cycloalkyl, heterocycloalkyl, hydroxyalkyl, or haloalkyloxy;
X is NR^{2′}, O, or S;
Z is N or CR^{2′}.

### CROSS REFERENCE TO RELATED APPLICATIONS

The present application claims priority from Continuation-in-Part application Ser. No. 11/375,259 filed Mar. 15, 2006 and Ser. No. 11/192,009 filed Jul. 29, 2005 and U.S. Provisional Application No. 60/612,794 filed Sep. 27, 2004, the contents of which are incorporated herein by reference in their entireties.

### BACKGROUND OF THE INVENTION

1. Field of the Invention

The present invention relates generally to compounds of the general formula (I), (Ia), (Ib), (Ic), (Ih), (II), or (III) and/or a stereoisomer thereof and/or a possible pharmaceutically acceptable salt thereof with an acid or a base, and/or a pharmaceutically acceptable prodrug of these compounds, for example, for use as a medicament. Compounds of the invention are exceptionally useful for the treatment of diseases associated with abnormal and hyperproliferation of cells in mammals, especially in humans. In particular, they are useful for the treatment of diseases characterized by a hyperproliferation of T-cells. The present invention relates to compounds which are suitable for the therapy of diseases that can be treated by modulating cellular pathways in eukaryotes, e.g. cancer, immunological or inflammatory disorders, and viral infections, to further processes for the preparation of these compounds, and to their use.

2. Description of Related Art

T-cell homeostasis is critical for the maintenance of immune tolerance. Defects in T-cell homeostasis can lead to autoimmune pathology. Autoimmune diseases include a large spectrum of clinically distinct entities that share a common aetiology, a misguided, self-directed immune response. This immune response can also be the consequence of an organ transplant. Evidence suggests a prime role of T-cell reactivity in autoimmune diseases. Measuring proliferative responses in T-lymphocytes is a widely used assay to measure immune competence (Killestein, J. et al. J. Neuroimmunol. 133,217-24, 2002).

We used a nonradioactive technique for the measurement of in vitro T-cell proliferation (Messele, T. et al. Clinical and Diagnostic Laboratory Immunology 687-692, 2000). Peripheral blood mononuclear cells (PBMCs) were isolated from human blood obtained from volunteer donators. PBMCs were isolated by centrifugation in ACCUSPIN tubes using HISTOPAQUE. PBMCs were stimulated with PHA and cell proliferation was measured with a Roche colorimetric BromUridin incorporation ELISA kit.

Regulation of the immune response is controlled by a variety of signalling pathways such as T-cell or TNF receptor signalling (Chen, G. et al. Science 296, 1634-1635, 2002). To further characterize targets of compounds which we found active in the T-cell proliferation assay, we tested the compounds on their ability to inhibit the human proteasome.

The major neutral proteolytic activity in the cytosol and nucleus is the proteasome, a 20S (700 kDa) particle with multiple peptidase activities. The continual turnover of cellular proteins by the ubiquitin-proteasome pathway is used by the immune system to screen for the presence of abnormal intracellular proteins (Dantuma, N. P. et al. Nat. Biotechnol. 2000, 18(5), 538-43; Goldberg, A L. et al. Nature 357, 375, 1993). The ubiquitin-proteasome pathway plays an essential role in the regulation of NF-κB activity, being responsible for the degradation of the inhibitor IκB-α. In order to be targeted for degradation by the proteasome, IκB-α must first undergo selective phosphorylation at serine residues 32 and 36, followed by ubiquitinylation (Chen, Z J. et al. Cell 84, 853-862, 1996; Brown, K. et al. Science 267, 1485, 1995). NF-κB, a transcription factor, regulates the transcription of an important set of genes, involved in inflammatory responses (Baeuerle, P A. et al. Cell 87, 1, 13-20, 1996). Proteasome inhibitors block IκB-α degradation and NF-κB activation (Traeckner et al. EMBO J. 13, 5433, 1994).

Literature describing proteasome inhibitors has been described in reviews (Adams, J. et al. Ann. Rev. Med. Chem. 31, 279-288, 1996) and in patents U.S. Pat. No. 6,117,887, U.S. Pat. No. 5,834,487, WO 00/004954, WO 00/04954, WO 00/170204, WO 00/33654, WO 00/64863, WO 00/114324, WO 99/15183, WO 99/37666.

Here we describe novel chemical entities with proteasome inhibitory activity. NF-κB (Nuclear Factor-κB) is a eukaryotic transcription factor of the rel family, which is located in the cytoplasm in an inactive complex, as a homo- or heterodimer. Predominantly it exists as a heterodimer composed of p50 and p65 subunits, bound to inhibitory proteins of the IκB family, usually IκB-α (D. Thanos et al., Cell 80, 529, 1995). NF-κB is activated in response to different stimuli, among which inflammatory cytokines, UV radiation, phorbol esters, bacterial and viral infections. Stimulation triggers the release of NF-κB from IκB in consequence of the phosphorylation and the following degradation of the IκB-α protein (P. A. Baeuerle et al., Annu. Rev. Immunol. 12, 141, 1995) by the proteasome. Once it is set free, NF-κB translocates in the nucleus where it binds to the DNA at specific κB-sites and induces the transcription of a variety of genes encoding proteins involved in controlling the immune and inflammatory responses, amongst others interleukins, TNF-α, the NO-synthase and the cyclooxygenase 2 (S. Grimm et al., J. Biochem. 290, 297, 1993). Accordingly, NF-κB is considered an early mediator of the immune and inflammatory responses and it is involved in the control of cell proliferation and in the pathogenesis of various human diseases, such as rheumatoid arthritis (H. Beker et al., Clin. Exp. Immunol. 99, 325, 1995), ischemia (A. Salminen et al., Biochem. Biophys. Res. Comm. 212, 939, 1995), arteriosclerosis (A. S. Baldwin, Annals Rev. Immunol. 212, 649, 1996), as well as in the pathogenesis of AIDS. Inhibition of NF-κB mediated gene transcription can be accomplished through inhibition of phosphorylation of the inhibitory protein IκB, inhibition of IκB degradation, inhibition of NF-κB (p50/p65) nuclear translocation, the inhibition of NF-κB-DNA binding or NF-κB-mediated DNA transcription (J. C. Epinat et al., Oncogene 18, 6896, 1999).

### SUMMARY OF THE INVENTION

The present invention relates to compounds of the general formula (I) or a salt or a physiologically functional derivative or a stereoisomer thereof,

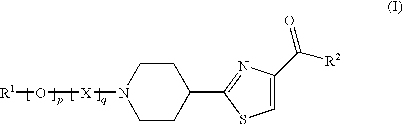

wherein
R¹ is independently hydrogen, alkyl, cycloalkyl, hydroxyalkyl, haloalkyl, haloalkyloxy, aryl, substituted aryl, heteroaryl, substituted heteroaryl, arylalkyl or substituted arylalkyl;
R² is independently —NR³R⁴,

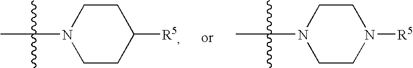

R³ is independently alkyl, cycloalkyl, alkoxy, alkylamine, —OH, —SH, alkylthio, hydroxyalkyl, haloalkyl, haloalkyloxy, aryl or heteroaryl;
R⁴ is independently alkyl, cycloalkyl, alkoxy, alkylamine, alkylthio, hydroxyalkyl, haloalkyl, haloalkyloxy, aryl or heteroaryl;
R⁵ is independently H, COR⁶, CO₂R⁶, SOR⁶, SO₂R⁶, SO₃R⁶, alkyl, cycloalkyl, alkoxy, —NH₂, alkylamine, —NR⁷COR⁶, halogen, —OH, —SH, alkylthio, hydroxyalkyl, haloalkyl, haloalkyloxy, aryl or heteroaryl;
R⁶ is independently H, alkyl, cycloalkyl, —NH₂, alkylamine, aryl or heteroaryl;
R⁷ is independently H, alkyl, cycloalkyl, alkoxy, —OH, —SH, alkylthio, hydroxyalkyl, aryl, or heteroaryl;
p is 0, or 1;
q is 0, or 1;
X is CO or SO₂;

wherein

an alkyl group, if not stated otherwise, denotes a linear or branched C₁-C₆-alkyl, preferably a linear or branched chain of one to five carbon atoms, a linear or branched C₂-C₆-alkenyl or a linear or branched C₂-C₆-alkinyl group, which can optionally be substituted by one or more substituents R′;

the C₁-C₆-alkyl, C₂-C₆-alkenyl and C₂-C₆-alkinyl residue may be selected from the group comprising —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C(R′)₃, —C₂(R′)₅, —CH₂—C(R′)₃, —C₃(R′)₇, —C₂H₄—C(R′)₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkyloxy, aryl, arylalkyl or heteroaryl;

R″ is independently H, haloalkyl, hydroxyalkyl, alkyl, cycloalkyl, aryl, heteroaryl or aminoalkyl;

a cycloalkyl group denotes a non-aromatic ring system containing three to eight carbon atoms, preferably four to eight carbon atoms, wherein one or more of the carbon atoms in the ring can be substituted by a group E, E being O, S, SO, SO₂, N, or NR″, R″ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅, morpholine-4-yl, piperazinyl, 1-alkylpiperazine-4-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an S-alkyl group, the alkyl group being as defined above;

a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R_{10″}, —C₂(R¹⁰)₅, —CH₂—C(R¹⁰)₃, —CH₂—CR¹⁰(R^{10′})₂, —CH₂—CR¹⁰(R^{10′})R^{10″}, —C₃(R¹⁰)₇, or —C₂H₄—C(R¹⁰)₃, wherein R¹⁰, R^{10′}, R^{10″} represent F, Cl, Br or I, preferably F;

a hydroxyalkyl group denotes an HO-alkyl group, the alkyl group being as defined above;

a haloalkyloxy group denotes an alkoxy group which is substituted by one to five halogen atoms, the alkyl group being as defined above; the haloalkyloxy group is preferably a) —OC(R¹⁰)₃, —OCR¹⁰(R^{10′})₂, —OCR¹⁰(R^{10′})R^{10″}, —OC₂(R¹⁰)₅, —OCH₂—C(R¹⁰)₃, —OCH₂—CR¹⁰(R^{10′})₂, —OCH₂—CR¹⁰(R^{10′})R^{10″}, —OC₃(R¹⁰)₇ or —OC₂H₄—C(R¹⁰)₃, wherein R¹⁰, R^{10′}, R^{10″} represent F, Cl, Br or I, preferably F;

a hydroxyalkylamino group denotes an (HO-alkyl)₂—N— group or HO-alkyl-NH— group, the alkyl group being as defined above;

an alkylamino group denotes an HN-alkyl or N-dialkyl group, the alkyl group being as defined above;

a halogen group is chlorine, bromine, fluorine or iodine;

an aryl group denotes an aromatic group having five to fifteen carbon atoms, which can optionally be substituted by one or more substituents R′, where R′ is as defined above; the aryl group is preferably a phenyl group, -o-C₆H₄—R′, -m-C₆H₄—R′, -p-C₆H₄—R′, 1-naphthyl, 2-naphthyl, 1-anthracenyl or 2-anthracenyl;

a heteroaryl group denotes a 5- or 6-membered heterocyclic group which contains at least one heteroatom like O, N, S. This heterocyclic group can be fused to another ring. For example, this group can be selected from a thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1-imidazolyl, 2-imidazolyl, 1,2,5-thiadiazol-4-yl, 4-imidazolyl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 3-pyridazinyl, 4-pyridazinyl, 2-pyrazinyl, 1-pyrazolyl, 3-pyrazolyl, 4-pyrazolyl, 1H-tetrazol-2-yl, 1H-tetrazol-3-yl, tetrazolyl, 2-indolyl, 3-indolyl, 2-indolinyl, 3-indolinyl, benzo[b]furanyl, benzo[b]thiophenyl, benzimidazolyl, benzothiazolyl, quinazolinyl, quinoxazolinyl, quinolinyl, tetrahydroquinolinyl, isoquinolinyl, tetrahydro-thieno[3,4-d]imidazol-2-one, pyrazolo[5,1-c][1,2,4]triazine, or tetrahydroisoquinolinyl group. This heterocyclic group can optionally be substituted by one or more substituents R′, wherein R′ is as defined above.

The invention also provides a pharmaceutical composition comprising a compound of formula (I), in free form or in the form of pharmaceutically acceptable salts and physiologically functional derivatives, together with a pharmaceutically acceptable diluent or carrier therefore.

The term “physiologically functional derivative” as used herein refers to compounds which are not pharmaceutically active themselves but which are transformed into their pharmaceutical active form in vivo, i.e. in the subject to which the compound is administered. Examples of physiologically functional derivatives are prodrugs.

In addition, the present invention provides methods for preparing the compounds of the invention such as compounds of formula (I).

Additional objects, features and advantages of the invention will be set forth in the description which follows, and in part, will be obvious from the description, or may be learned by practice of the invention. Objects, features and advantages of the invention may be realized and obtained by means of the instrumentalities and combination particularly pointed out in the appended claims.

### DETAILED DESCRIPTION OF A PREFERRED EMBODIMENT

The compounds of formula (I) may be obtained via various methods.

Piperidin-4yl-thiazole-4-carboxamide can be prepared by various methods described in the literature. One such example is the oxidation of the appropriate 2,5-dihydrothiazoles as described in Houben-Weyl, 2002, 730. The dihydrothiazoles can also synthesised by methods described in the same reference or described in You, S., Razavi, H., Kelly, J. W. Angew. Chem. 2003, 115, 87 or Katritzky, A R., Cai, C., Suzuki, K., Singh, S K. J. Org. Chem. 2004, 69, 811-814 and references in both papers. Alternative methods were described by Yasuchika, S. et. al. Heterocycles, Vol. 57, No. 5, 2002, which are incorporated herein by reference in their entireties.

In a preferred embodiment of the invention, in the compounds of formula (I),

R¹ is:

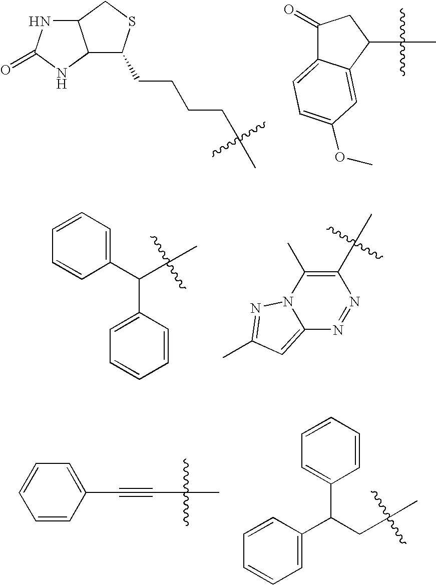

In a preferred embodiment of the invention, in the compounds of formula (I), R⁵ is:

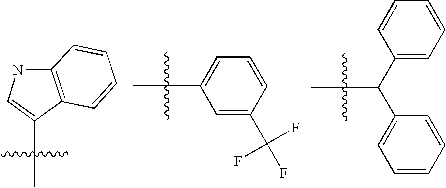

In another preferred embodiment, in the compounds of formula (I), R³, R⁴ and R⁷ is H.

In a preferred embodiment of the invention, in the compounds of formula (I), R² is:

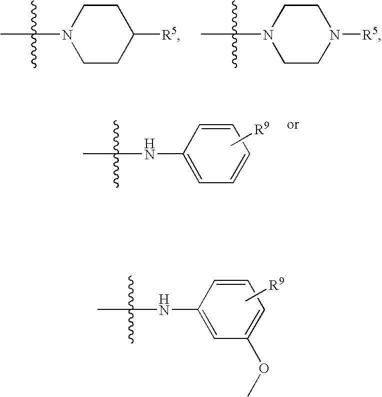

In a preferred embodiment of the invention, in the compounds of formula (I), R¹ is substituted aryl, p is 0, q is 1, X is CO, and

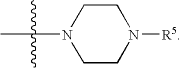

R² is

In a preferred embodiment of the invention, in the compounds of formula (I), R¹ is benzyl, p is 0, q is 0, and

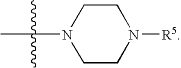

R² is

The present invention relates to compounds of the general formula (Ia) or a pharmaceutically acceptable salts thereof with an acid or a base, or pharmaceutically acceptable prodrugs or a stereoisomer thereof,

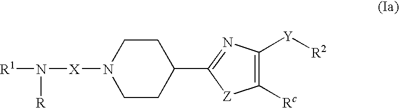

wherein
R is independently hydrogen, alkyl, cycloalkyl, hydroxyalkyl, haloalkyl, haloalkyloxy, aryl, or heteroaryl;
R¹ is independently alkyl, cycloalkyl, hydroxyalkyl, haloalkyl, haloalkyloxy, aryl, or heteroaryl;
X is CO, CS or SO₂;
Y is CO, CS or SO₂;
Z is NR^{2′}, S, or O;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R^{e} is independently H, OH, SH, NROR¹, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R⁷, R^{7′} independently represent H, alkyl, cycloalkyl, haloalkyl, hydroxyalkyl, hydroxyalkylamino, alkylamino, heteroaryl or aryl;
R⁸ is H, NH₂, alkyl, cycloalkyl, haloalkyl, hydroxyalkyl, hydroxyalkylamino, alkylamino, heteroaryl or aryl;
R^{e} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}benzthiazolyl, —C(NR^{4″})NR^{4′}benz-oxazolyl, or heteroaryl;

R^{4′}, R^{4″}, R^{5′} independently represent H, alkyl, cycloalkyl, haloalkyl, hydroxyalkyl, hydroxyalkylamino, alkylamino, —C(NR⁷)NR^{7′}R⁸, —(CH₂)ₚaryl, —CH₂)ₚNR⁷R⁸, —C(O)NR⁷R⁸, —N═CR⁷R⁸, —NR⁷C(O)R⁸, halogen, heteroaryl, or aryl
p is 1 to 6;
q is 1 to 6;
R² is independently

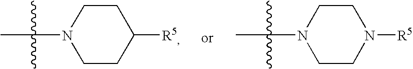

R⁵ is independently H, COR⁶, CO₂R⁶, SOR⁶, SO₂R⁶, SO₃R⁶, alkyl, cycloalkyl, alkoxy, —NH₂, alkylamine, —NR⁷COR⁶, halogen, —OH, —SH, alkylthio, hydroxyalkyl, haloalkyl, haloalkyloxy, aryl or heteroaryl;
R⁶ is independently H, alkyl, cycloalkyl, amino, haloalkyl, hydroxyalkyl, hydroxyalkylamino, alkylamino, aryl or heteroaryl;

wherein

an alkyl group, if not stated otherwise, denotes a linear or branched C₁-C₆-alkyl, preferably a linear or branched chain of one to five carbon atoms, a linear or branched C₂-C₆-alkenyl or a linear or branched C₂-C₆-alkinyl group, which can optionally be substituted by one or more substituents R′;

the C₁-C₆-alkyl, C₂-C₆-alkenyl and C₂-C₆-alkinyl residue may be selected from the group comprising —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C(R′)₃, —C₂(R′)₅, —CH₂—C(R′)₃, —C₃(R′)₇, —C₂H₄—C(R′)₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH—CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;

R″ is independently H, haloalkyl, hydroxyalkyl, alkyl, cycloalkyl, aryl, or heteroaryl;

a cycloalkyl group denotes a non-aromatic ring system containing three to eight carbon atoms, preferably four to eight carbon atoms, wherein one or more of the carbon atoms in the ring can be substituted by a group E, E being O, S, SO, SO₂, N, or NR″, R″ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅, morpholine-4-yl, piperazinyl, 1-alkylpiperazine-4-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an 5-alkyl group, the alkyl group being as defined above;

a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂—C(R¹⁰)₃, —CH₂—CR¹⁰(R^{10′})₂, —CH₂—CR¹⁰(R^{10′})R^{10″}, —C₃(R¹⁰)₇, or —C₂H₄—C(R¹⁰)₃, wherein R¹⁰, R^{10′}, R^{10″} represents F, Cl, Br or I, preferably F;

a hydroxyalkyl group denotes an HO-alkyl group, the alkyl group being as defined above;

a haloalkyloxy group denotes an alkoxy group which is substituted by one to five halogen atoms, the alkyl group being as defined above; the haloalkyloxy group is preferably a —OC(R¹⁰)₃, —OCR¹⁰(R^{10′})₂, —OCR¹⁰(R^{10′})R^{10″}, —OC₂(R¹⁰)₅, —OCH₂—C(R¹⁰)₃, —OCH₂—CR¹⁰(R^{10′})₂, —OCH₂—CR¹⁰(R^{10′})R^{10″}, —OC₃(R¹⁰)₇ or —OC₂H₄—C(R¹⁰)₃, wherein R¹⁰, R^{10′}, R^{10″} represent F, Cl, Br or I, preferably F;

a hydroxyalkylamino group denotes an (HO-alkyl)₂-N— group or HO-alkyl-NH— group, the alkyl group being as defined above;

an alkylamino group denotes an HN-alkyl or N-dialkyl group, the alkyl group being as defined above;

a halogen group is chlorine, bromine, fluorine or iodine;

an aryl group denotes an aromatic group having five to fifteen carbon atoms, which can optionally be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl;

a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, tetra-hydrothieno[3,4-d]imidazol-2-one, pyrazolo[5,1-c][1,2,4]triazine, 2,3-dihydrobenzo[1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, imidazopyridazine, imidazopyrimidine, imidazopyridine, imidazolotriazine, triazolotriazine, triazolopyridine, triazolopyrazine, triazolopyrimidine, or 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

The present invention relates to compounds of the general formula (Ib) or pharmaceutically acceptable salts thereof with an acid or a base, or pharmaceutically acceptable prodrugs or a stereoisomer thereof,

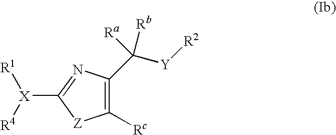

wherein
R¹ is —C(O)R⁷, —C(O)CHR⁷R⁸, —C(O)NR⁷R⁸, —C(O)OR⁷, —R⁷C(O)R⁸, or —C(S)R⁷;
R⁹ independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl hydroxyalkylamino, alkoxy, alkylthio, —O(CH₂)ₚ[O(CH₂)ₚ]_{q}OCH₃, —C(NR^{4″})NR^{4′}-benzimidazolyl, —C(NR^{4″})NR^{4′}benzthiazolyl, —C(NR^{4″})NR^{4′}benzoxazolyl, —(CH₂)ₚNR⁷COR⁸, or heteroaryl;
R⁴ is H, alkyl, cycloalkyl, haloalkyl, hydroxyalkyl, hydroxyalkylamino, alkylamino, heteroaryl, aryl,
or R¹ and R⁴ together with the X to which they are attached form a 3 to 8 membered saturated or at least partially unsaturated monocyclic or polycyclic ring system, wherein at least one ring atom is a heteroatom selected from O, N, and S, and the ring optionally has one or more substituents R⁹;
X is N, or CR^{2′};
Y is CO, CS or SO₂;
Z is NR^{2″}, S, or O;
R^{2″} is H, alkyl, —C(O)NR⁷, —C(O)R^{e}, cycloalkyl, haloalkyl, hydroxyalkyl, hydroxyalkylamino, alkylamino, heteroaryl, or aryl;
R^{2′} is H, alkyl, —C(O)NR^{4′}, —C(O)R^{4′}, cycloalkyl, haloalkyl, hydroxyalkyl, hydroxy-alkylamino, alkylamino, heteroaryl, or aryl;
R^{4′}, R^{4″}, R^{5′} independently represent H, alkyl, cycloalkyl, haloalkyl, hydroxyalkyl, hydroxyalkylamino, alkylamino, —C(NR⁷)NR^{7′}R⁸, —(CH₂)ₚaryl, —CH₂)ₚNR⁷R⁸, —C(O)NR⁷R⁸, —N═CR⁷R⁸, —NR⁷C(O)R⁸, halogen, heteroaryl, or aryl
p is 1 to 6;
q is 1 to 6;
R^{a} is independently H, OH, SH, NH₂, alkyl, cycloalkyl, hydroxyalkyl, haloalkyl, haloalkyloxy, alkoxy, alkylamino, hydroxyalkylamino, halogen, aryl, or heteroaryl;
R^{b} is independently H, OH, SH, NH₂, alkyl, cycloalkyl, hydroxyalkyl, haloalkyl, haloalkyloxy, alkoxy, alkylamino, hydroxyalkylamino, halogen, aryl, or heteroaryl;
R^{c} is independently H, OH, SH, NR^{4′}OR^{5′}, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R⁷, R^{7′} independently represent H, alkyl, cycloalkyl, haloalkyl, hydroxyalkyl, hydroxyalkylamino, alkylamino, heteroaryl or aryl;
R⁸ is H, NH₂, alkyl, cycloalkyl, haloalkyl, hydroxyalkyl, hydroxyalkylamino, alkylamino, heteroaryl or aryl;
R^{e} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, hydroxyalkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, aryl or heteroaryl;
R² is independently

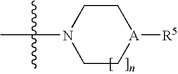

A is N, O, or CR^{2′};
R⁵ is independently H, SOR⁷, SO₂R⁷, SO₃R⁷, —C(O)R⁷, —C(O)CHR⁷R⁸, —C(O)NR⁷R⁸, —C(O)OR⁷, —R⁷C(O)R⁸, —C(S)R⁷, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R^{7′}, alkyl, cycloalkyl, alkoxy, —NH₂, alkylamino, hydroxyalkylamino, halogen, —OH, —SH, alkylthio, hydroxyalkyl, haloalkyl, haloalkyloxy, aryl or heteroaryl;
n is 0 to 2;

wherein

an alkyl group, if not stated otherwise, denotes a linear or branched C₁-C₆-alkyl, preferably a linear or branched chain of one to five carbon atoms, a linear or branched C₂-C₆-alkenyl or a linear or branched C₂-C₆-alkinyl group, which can optionally be substituted by one or more substituents R′;

the C₁-C₆-alkyl, C₂-C₆-alkenyl and C₂-C₆-alkinyl residue may be selected from the group comprising —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C(R′)₃, —C₂(R′)₅, —CH₂—C(R′)₃, —C₃(R′)₇, —C₂H₄—C(R′)₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;

R″ is independently H, haloalkyl, hydroxyalkyl, alkyl, cycloalkyl, aryl, or heteroaryl;

a cycloalkyl group denotes a non-aromatic ring system containing three to eight carbon atoms, preferably four to eight carbon atoms, wherein one or more of the carbon atoms in the ring can be substituted by a group E, E being O, S, SO, SO₂, N, or NR″, R″ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅, morpholine-4-yl, piperazinyl, 1-alkylpiperazine-4-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an S-alkyl group, the alkyl group being as defined above;

a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂—C(R¹⁰)₃, —CH₂—CR¹⁰(R^{10′})₂, —CH₂—CR¹⁰(R^{10′})R^{10″}, —C₃(R¹⁰)₇, or —C₂H₄—C(R¹⁰)_{3,} wherein R¹⁰, R^{10′}, R^{10″} represent F, Cl, Br or I, preferably F;

a hydroxyalkyl group denotes an HO-alkyl group, the alkyl group being as defined above;

a haloalkyloxy group denotes an alkoxy group which is substituted by one to five halogen atoms, the alkyl group being as defined above; the haloalkyloxy group is preferably a —OC(R¹⁰)₃, —OCR¹⁰(R^{10′})₂, —OCR¹⁰(R^{10′})R^{10″}, —OC₂(R¹⁰)₅, —OCH₂—C(R¹⁰)₃, —OCH₂—CR¹⁰(R^{10′})₂, —OCH₂—CR¹⁰(R^{10′})R^{10″}, —OC₃(R¹⁰)₇ or —OC₂H₄—C(R¹⁰)₃, wherein R¹⁰, R^{10′}, R^{10″} represent F, Cl, Br or I, preferably F;

a hydroxyalkylamino group denotes an (HO-alkyl)₂-N— group or HO-alkyl-NH— group, the alkyl group being as defined above;

an alkylamino group denotes an HN-alkyl or N-dialkyl group, the alkyl group being as defined above;

a halogen group is chlorine, bromine, fluorine or iodine;

an aryl group denotes an aromatic group having five to fifteen carbon atoms, which can optionally be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl;

a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 6-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, tetrahydro-thieno[3,4-d]imidazol-2-one, pyrazolo[5,1-c][1,2,4]triazine, imidazopyridazine, imidazopyrimidine, imidazopyridine, imidazolotriazine, triazolotriazine, 2,3-dihydrobenzo[1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, triazolopyridine, triazolopyrazine, triazolopyrimidine, 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

The present invention relates to compounds of the general formula (Ic) or pharmaceutically acceptable salts thereof with an acid or a base, or pharmaceutically acceptable prodrugs or a stereoisomer thereof,

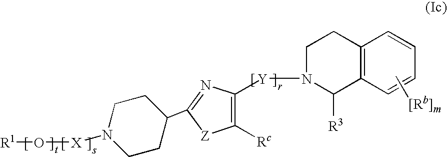

wherein
R¹ independently represents H, alkyl, cycloalkyl, hydroxyalkyl, haloalkyl, haloalkyloxy, aryl, or heteroaryl;
X is CO, CS or SO₂;
Y is CO, CS or SO₂;
Z is NR^{2″}, S, or O;
R^{2″} is H, alkyl, —C(O)NR⁷, —C(O)R^{e}, cycloalkyl, haloalkyl, hydroxyalkyl, hydroxyalkylamino, alkylamino, heteroaryl, or aryl;
R⁴′, R^{4″}, R^{5′} independently represent H, alkyl, cycloalkyl, haloalkyl, hydroxyalkyl, hydroxyalkylamino, alkylamino, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, halogen, heteroaryl, or aryl
p is 1 to 6;
q is 1 to 6;
m is 0 to 4;
r is 0, or 1;
t is 0, or 1;
s is 0, or 1;
R^{b} is independently H, OH, SH, NR^{4′}OR^{5′}, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{c} is independently H, OH, SH, NR^{4′}OR^{5′}, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R⁷, R^{7′} independently represent H, alkyl, cycloalkyl, haloalkyl, hydroxyalkyl, hydroxyalkylamino, alkylamino, heteroaryl or aryl;
R⁸ is H, NH₂, alkyl, cycloalkyl, haloalkyl, hydroxyalkyl, hydroxyalkylamino, alkylamino, heteroaryl or aryl;
R^{e} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, hydroxyalkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benz-oxazolyl, aryl or heteroaryl;
R³ is independently H, OH, SH, NR^{4′}OR^{5′}, NH₂, hydroxyalkylamino, alkylamino, halogen, CONR^{d}R^{e}, alkoxy, alkyl, cycloalkyl, hydroxyalkyl, haloalkyl, haloalkyloxy, aryl, or heteroaryl;

an alkyl group, if not stated otherwise, denotes a linear or branched C₁-C₆-alkyl, preferably a linear or branched chain of one to five carbon atoms, a linear or branched C₂-C₆-alkenyl or a linear or branched C₂-C₆-alkinyl group, which can optionally be substituted by one or more substituents R′;

the C₁-C₆-alkyl, C₂-C₆-alkenyl and C₂-C₆-alkinyl residue may be selected from the group comprising —CH₃, —C₂H₅, —CH═CH₂, —C≡CH, —C₃H₇, —CH(CH₃)₂, —CH₂—CH═CH₂, —C(CH₃)═CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C(R′)₃, —C₂(R′)₅, —CH₂—C(R′)₃, —C₃(R′)₇, —C₂H₄—C(R′)₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;

R″ is independently H, haloalkyl, hydroxyalkyl, alkyl, cycloalkyl, aryl, or heteroaryl;

a cycloalkyl group denotes a non-aromatic ring system containing three to eight carbon atoms, preferably four to eight carbon atoms, wherein one or more of the carbon atoms in the ring can be substituted by a group E, E being O, S, SO, SO₂, N, or NR″, R″ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅, morpholine-4-yl, piperazinyl, 1-alkylpiperazine-4-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an S-alkyl group, the alkyl group being as defined above;

an haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂—C(R¹⁰)₃, —CH₂—CR¹⁰(R^{10′})₂, —CH₂—CR¹⁰(R^{10′})R^{10″}, —C₃(R¹⁰)₇, or —C₂H₄—C(R¹⁰)₃, wherein R¹⁰, R^{10′}, R^{10″} represent F, Cl, Br or I, preferably F;

a hydroxyalkyl group denotes an HO-alkyl group, the alkyl group being as defined above;

an haloalkyloxy group denotes an alkoxy group which is substituted by one to five halogen atoms, the alkyl group being as defined above; the haloalkyloxy group is preferably a) —OC(R¹⁰)₃, —OCR¹⁰(R^{10′})₂, —OCR¹⁰(R^{10′})R^{10″}, —OC₂(R¹⁰)₅, —OCH₂—C(R¹⁰)₃, —OCH₂—CR¹⁰(R^{10′})₂, —OCH₂—CR¹⁰(R^{10′})R^{10″}, —OC₃(R¹⁰)₇ or —OC₂H₄—C(R¹⁰)₃, wherein R¹⁰, R^{10′}, R^{10″} represent F, Cl, Br or I, preferably F;

a hydroxyalkylamino group denotes an (HO-alkyl)₂-N— group or HO-alkyl-NH— group, the alkyl group being as defined above;

an alkylamino group denotes an HN-alkyl or N-dialkyl group, the alkyl group being as defined above;

a halogen group is chlorine, bromine, fluorine or iodine;

an aryl group denotes an aromatic group having five to fifteen carbon atoms, which can optionally be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl;

a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 6-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydro-thieno[3,4-d]imidazol-2-one, pyrazolo[5,1-c][1,2,4]triazine, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, imidazopyridazine, imidazopyrimidine, imidazopyridine, imidazolotriazine, triazolotriazine, triazolopyridine, triazolopyrazine, triazolopyrimidine, 2,3-dihydrobenzo[1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

The present invention also relates to compounds of the general formula (III) or pharmaceutically acceptable salts thereof with an acid or a base, or pharmaceutically acceptable prodrugs or a stereoisomer thereof,

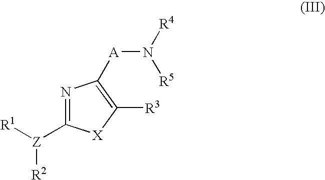

wherein
R¹ is —C(O)R^{7a}, —C(O)CHR⁷R⁸, —C(O)NR⁷R⁸, —C(O)OR⁷, —R⁷C(O)R⁸, or —C(S)R^{7b};
R² is H, alkyl, cycloalkyl, heterocycloalkyl, haloalkyl, hydroxyalkyl, hydroxyalkylamino, alkylamino, heteroaryl,
or R¹ and R² together with the N-atom or the C-atom to which they are attached form a 3 to 8 membered saturated or at least partially unsaturated monocyclic or polycylic ring system, wherein at least one or more of the carbon atoms in the ring is a heteroatom selected from O, N, and S, and the ring can be substituted by one or more R^{9;}
R³ is H, —C(O)NR^{a}R^{b}, halogen, alkyl, haloalkyl, aryl, heteroaryl, OH, SH, NR^{4′}OR^{5′}, NH₂, hydroxyalkylamino, alkylamino, alkoxy, cycloalkyl, heterocycloalkyl, hydroxyalkyl, or haloalkyloxy;
R⁴ is H, OH, SH, NH₂, alkoxy, haloalkoxy, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, heteroaryl, or aryl;
R⁵ is halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, heteroaryl, or aryl;
R^{a} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, heteroaryl, or aryl;
R^{b} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, alkylamino, alkoxy, alkylthio, halogen, haloalkyl, haloalkyloxy, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, hydroxyalkyl, hydroxycycloalkyl, hydroxyalkylamino, heterocycloalkyl, aryl or heteroaryl;
R^{4′}, R^{4″}, R^{5′} independently are H, halogen, alkyl, —C(NR⁷)NR^{7′}R⁸, —(CH₂)ₚaryl, haloalkyl, —CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino heteroaryl, or aryl;
R^{7a} is cycloalkyl, haloalkyl, hydroxyalkyl, hydroxyalkylamino, heteroaryl, or aryl;
R^{7b} is H, halogen, alkyl, cycloalkyl, heterocycloalkyl, haloalkyl, hydroxyalkyl, hydroxyalkylamino, heteroaryl, or aryl;
A is CO or SO₂;
X is NR^{2′}, O, or S;
Z is N or CR^{2′};
R^{2′} is H, alkyl, —C(O)NR⁷, —C(O)R^{b}, cycloalkyl, heterocycloalkyl, haloalkyl, hydroxyalkyl, hydroxyalkylamino, alkylamino, heteroaryl, or aryl;
p is 1 to 6;
q is 1 to 6;
R⁹ independently represents H, —CN, —OH, —SH, alkoxy, alkylthio, —CO₂R^{4′}, —C(O)R^{4a}, —C(O)NR⁷R⁸, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, halogen, haloalkyl, alkylamino, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, hydroxycycloalkyl, hydroxyalkylamino, haloalkyloxy, heterocycloalkyl, —(CH₂)ₚNR⁷COR⁸, aryl, or heteroaryl;
R^{4a} is H, C₁-C₆-alkyl, C₂-C₆-alkenyl, cycloalkyl, haloalkyl, hydroxyalkyl, hydroxyalkylamino, alkylamino, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, halogen, heteroaryl, or aryl;

wherein

an C₁-C₆-alkyl group, if not stated otherwise, denotes a linear or branched C₁-C₆-alkyl, preferably a linear or branched chain of one to five carbon atoms, which can optionally be substituted by one or more substituents R′;

an C₂-C₆-alkenyl group, if not stated otherwise, denotes a linear or branched C₂-C₆-alkenyl, preferably a linear or branched chain of two to six carbon atoms, which can optionally be substituted by one or more substituents R′;

an alkyl group, if not stated otherwise, denotes a linear or branched C₁-C₆-alkyl, preferably a linear or branched chain of one to six carbon atoms, a linear or branched C₂-C₆-alkenyl or a linear or branched C₂-C₆-alkynyl group, which can be substituted by one or more substituents R′;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;

R″ is independently H, haloalkyl, hydroxyalkyl, alkyl, cycloalkyl, aryl, or heteroaryl;

a cycloalkyl group denotes a non-aromatic ring system containing three to eight carbon atoms, preferably four to eight carbon atoms, wherein one or more of the carbon atoms in the ring can be substituted by a group R′ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅;

a heterocycloalkyl group denotes a non-aromatic ring system containing two to ten carbon atoms and at least one heteroatom like O, N, or S, wherein one or more of the carbon atoms in the ring can be substituted by R′ being as defined above; preferred heterocycloalkyl groups are morpholine-4-yl, piperazinyl, 1-alkylpiperazine-4-yl, piperidinyl, pyrrolidinyl, azepane-1-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an S-alkyl group, the alkyl group being as defined above;

a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂C(R¹⁰)₃, —CH₂CR¹⁰(R^{10′})₂, —CH₂CR¹⁰(R^{10′})R^{10″}, —C₃(R¹⁰)₇, or —C₂H₄C(R¹⁰)₃, wherein R¹⁰, R^{10′}, R^{10″} represent F, Cl, Br or I, preferably F;

a hydroxyalkyl group denotes an HO-alkyl group, the alkyl group being as defined above;

a haloalkyloxy group denotes an alkoxy group which is substituted by one to five halogen atoms, the alkyl group being as defined above; the haloalkyloxy group is preferably a —OC(R¹⁰)₃, —OCR¹⁰(R^{10′})₂, —OCR¹⁰(R^{10′})R^{10″}, —OC₂(R¹⁰)₅, —OCH₂C(R¹⁰)₃, —OCH₂CR¹⁰(R^{10′})₂, —OCH₂CR¹⁰(R^{10′})R^{10″}, —OC₃(R¹⁰)₇, or —OC₂H₄C(R¹⁰)₃, wherein R¹⁰, R^{10′}, R^{10″} represent F, Cl, Br or I, preferably F;

a hydroxyalkylamino group denotes an (HO-alkyl)₂-N— group or HO-alkyl-NH— group, the alkyl group being as defined above;

an alkylamino group denotes an HN-alkyl or N-dialkyl group, the alkyl group being as defined above;

a halogen group is fluorine, chlorine, bromine, or iodine;

an aryl group denotes an aromatic group having five to fifteen carbon atoms, which can be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl, R′ being as defined above;

an arylamino group denotes an HN-aryl or N-diaryl group, the aryl group being as defined above;

a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 6-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydro-thieno[3,4-d]imidazol-2-one, pyrazolo[5,1-c][1,2,4]triazine, 2,3-dihydrobenzo[1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, imidazopyridazine, imidazo-pyrimidine, imidazopyridine, imidazolotriazine, triazolotriazine, triazolopyridine, triazolopyrazine, triazolopyrimidine, 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

A preferred embodiment of the invention, in the compounds of formula (III) are compounds of the general formula (Ih) or pharmaceutically acceptable salts thereof with an acid or a base, or pharmaceutically acceptable prodrugs or a stereoisomer thereof,

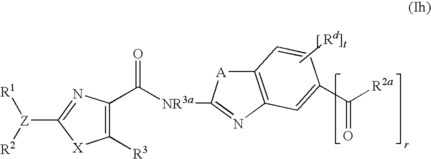

wherein
A is NR^{2′}, S or O;
t is 0 to 4;
r is 0, or 1;
R^{2a} is independently H, OH, SH, NH₂, alkyl, cycloalkyl, hydroxyalkyl, haloalkyl, haloalkyloxy, alkoxy, alkylamino, hydroxyalkylamino, halogen, aryl, or heteroaryl;
R^{3a} is independently H, OH, SH, NH₂, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, alkyl, cycloalkyl, hydroxyalkyl, haloalkyl, haloalkyloxy, alkoxy, alkylamino, hydroxyalkylamino, halogen, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl.
R¹ is —C(O)R^{7a}, —C(O)CHR⁷R⁸, —C(O)NR⁷R⁸, —C(O)OR⁷, —R⁷C(O)R⁸, or —C(S)R^{7b};
R² is H, alkyl, cycloalkyl, heterocycloalkyl, haloalkyl, hydroxyalkyl, hydroxyalkylamino, alkylamino, or heteroaryl;
or R¹ and R² together with the N-atom or the C-atom to which they are attached form a 3 to 8 membered saturated or at least partially unsaturated monocyclic or polycylic ring system, wherein at least one or more of the carbon atoms in the ring is a heteroatom selected from O, N, and S, and the ring can be substituted by one or more R^{9;}
R^{4a} is H, C₁-C₆-alkyl, C₂-C₆-alkenyl, cycloalkyl, haloalkyl, hydroxyalkyl, hydroxyalkylamino, alkylamino, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, halogen, heteroaryl, or aryl;
R³ is H, —C(O)NR^{a}R^{b}, halogen, alkyl, haloalkyl, aryl, heteroaryl, OH, SH, NR^{4′}OR^{5′}, NH₂, hydroxyalkylamino, alkylamino, alkoxy, cycloalkyl, heterocycloalkyl, hydroxyalkyl, or haloalkyloxy;
R^{a} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, heteroaryl, or aryl;
R^{b} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, alkylamino, alkoxy, alkylthio, halogen, haloalkyl, haloalkyloxy, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, hydroxyalkyl, hydroxycycloalkyl, hydroxyalkylamino, heterocycloalkyl, aryl or heteroaryl;
R^{4′}, R^{4″}, R^{5′} independently are H, halogen, alkyl, —C(NR⁷)NR^{7′}R⁸, —(CH₂)ₚaryl, haloalkyl, —CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino heteroaryl, or aryl;
R^{7a} is cycloalkyl, haloalkyl, hydroxyalkyl, hydroxyalkylamino, heteroaryl, or aryl;
R^{7b} is H, halogen, alkyl, cycloalkyl, heterocycloalkyl, haloalkyl, hydroxyalkyl, hydroxyalkylamino, heteroaryl, or aryl;
X is NR^{2′}, O, or S;
Z is N or CR^{2′};
R^{2′} is H, alkyl, —C(O)NR⁷, —C(O)R^{b}, cycloalkyl, heterocycloalkyl, haloalkyl, hydroxyalkyl, hydroxyalkylamino, alkylamino, heteroaryl, or aryl;
p is 1 to 6;
q is 1 to 6;
R⁹ independently represents H, —CN, —OH, —SH, alkoxy, alkylthio, —CO₂R^{4′}, —C(O)R^{4a}, —C(O)NR⁷R⁸, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, halogen, haloalkyl, alkylamino, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, hydroxycycloalkyl, hydroxyalkylamino, haloalkyloxy, heterocycloalkyl, —(CH₂)ₚNR⁷COR⁸, aryl, or heteroaryl;

wherein

a C₁-C₆-alkyl group, if not stated otherwise, denotes a linear or branched C₁-C₆-alkyl, preferably a linear or branched chain of one to five carbon atoms, which can optionally be substituted by one or more substituents R′;

a C₂-C₆-alkenyl group, if not stated otherwise, denotes a linear or branched C₂-C₆-alkenyl, preferably a linear or branched chain of two to six carbon atoms, which can optionally be substituted by one or more substituents R′;

an alkyl group, if not stated otherwise, denotes a linear or branched C₁-C₆-alkyl, preferably a linear or branched chain of one to six carbon atoms, a linear or branched C₂-C₆-alkenyl or a linear or branched C₂-C₆-alkynyl group, which can be substituted by one or more substituents R′;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;

R″ is independently H, haloalkyl, hydroxyalkyl, alkyl, cycloalkyl, aryl, or heteroaryl;

a cycloalkyl group denotes a non-aromatic ring system containing three to eight carbon atoms, preferably four to eight carbon atoms, wherein one or more of the carbon atoms in the ring can be substituted by a group R′ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅;

a heterocycloalkyl group denotes a non-aromatic ring system containing two to ten carbon atoms and at least one heteroatom selected from O, N, and S, wherein one or more of the carbon atoms in the ring can be substituted by R′ being as defined above; preferred heterocycloalkyl groups are morpholine-4-yl, piperazinyl, 1-alkylpiperazine-4-yl, piperidinyl, pyrrolidinyl, azepane-1-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an S-alkyl group, the alkyl group being as defined above;

a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂C(R¹⁰)₃, —CH₂CR¹⁰(R^{10′})₂, —CH₂CR¹⁰(R^{10′})R^{10″}, —C₃(R¹⁰)₇, or —C₂H₄C(R¹⁰)₃, wherein R¹⁰, R^{10′}, R^{10″} represent F, Cl, Br or I, preferably F;

a hydroxyalkyl group denotes an HO-alkyl group, the alkyl group being as defined above;

a haloalkyloxy group denotes an alkoxy group which is substituted by one to five halogen atoms, the alkyl group being as defined above; the haloalkyloxy group is preferably a —OC(R¹⁰)₃, —OCR¹⁰(R^{10′})₂, —OCR¹⁰(R^{10′})R^{10″}, —OC₂(R¹⁰)₅, —OCH₂C(R¹⁰)₃, —OCH₂CR¹⁰(R^{10′})₂, —OCH₂CR¹⁰(R^{10′})R^{10″}, —OC₃(R¹⁰)₇, or —OC₂H₄C(R¹⁰)₃, wherein R¹⁰, R^{10′}, R^{10″} represent F, Cl, Br or I, preferably F;

a hydroxyalkylamino group denotes an (HO-alkyl)₂-N— group or HO-alkyl-NH— group, the alkyl group being as defined above;

an alkylamino group denotes an HN-alkyl or N-dialkyl group, the alkyl group being as defined above;

a halogen group is fluorine, chlorine, bromine, or iodine;

an aryl group denotes an aromatic group having five to fifteen carbon atoms, which can be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl, R′ being as defined above;

an arylamino group denotes an HN-aryl or N-diaryl group, the aryl group being as defined above;

a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 6-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydro-thieno[3,4-d]imidazol-2-one, pyrazolo[5,1-c][1,2,4]triazine, 2,3-dihydrobenzo[1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, imidazopyridazine, imidazo-pyrimidine, imidazopyridine, imidazolotriazine, triazolotriazine, triazolopyridine, triazolopyrazine, triazolopyrimidine, 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

In formula (Ih), the following substituents are preferred, alone or in combination:

X is preferably S or O, more preferably S.

R^{3a} is preferably H.

In a preferred embodiment, Z is CR^{2′} and R¹ and R² together with the C-atom to which they are attached form a 6 membered saturated ring. In another preferred embodiment, Z is N and R¹ and R² together with the N-atom to which they are attached form a 6 membered saturated ring.

R⁹ is preferably heteroaryl, aryl or benzyl, more preferably heteroaryl. R⁹ is even more preferably thienopyrimidine, quinazoline, purine, pyrazolopyrimidine or triazolpyrimidine. Even more preferably, R⁹ is thienopyrimidine.

t is preferably 0.

r is preferably 1.

R^{2a} is preferably OH, alkyl, aryl or heteroaryl, more preferably alkyl, especially methyl.

The present invention relates also to compounds of the general formula (II) or pharmaceutically acceptable salts thereof with an acid or a base, or pharmaceutically acceptable prodrugs, or a stereoisomer thereof,

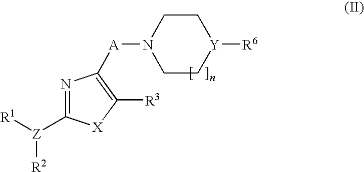

wherein
R¹ is —C(O)R⁷, —C(O)CHR⁷R⁸, —C(O)NR⁷R⁸, —C(O)OR⁷, —R⁷C(O)R⁸, or —C(S)R⁷;
R² is H, alkyl, cycloalkyl, heterocycloalkyl, haloalkyl, hydroxyalkyl, alkylamino, hydroxyalkylamino, or heteroaryl;
or R¹ and R² together with the N-atom or the C-atom to which they are attached form a 3 to 8 membered saturated or at least partially unsaturated monocyclic or polycyclic ring system, wherein at least one or more of the carbon atoms in the ring is a heteroatom selected from O, N, and S, and the ring can be substituted by one or more R⁹;
R³ is H, —C(O)NR^{a}R^{b}, halogen, alkyl, haloalkyl, aryl, heteroaryl, OH, SH, NR^{4′}OR^{5′}, NH₂, hydroxyalkylamino, alkylamino, alkoxy, cycloalkyl, heterocycloalkyl, hydroxyalkyl, or haloalkyloxy;
R^{a} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, heteroaryl, or aryl;
R^{b} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, 13 N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheterocycle, alkyl, cycloalkyl, alkylamino, alkoxy, alkylthio, halogen, haloalkyl, haloalkyloxy, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, hydroxyalkyl, hydroxyalkylamino, aryl, heterocycloalkyl, or heteroaryl;
R⁶ is halogen, —C(O)R⁷, —C(O)CHR⁷R⁸, —C(O)NR⁷R⁸, —C(O)OR⁷, —R⁷C(O)R⁸, —C(S)R⁷, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R^{7′}, alkyl, cycloalkyl, heterocycloalkyl, haloalkyl, hydroxyalkyl, hydroxyalkylamino, alkylamino, heteroaryl, or aryl;
R⁷, R^{7′}, R⁸ independently are H, halogen, alkyl, cycloalkyl, heterocycloalkyl, haloalkyl, hydroxyalkyl, hydroxyalkylamino, alkylamino, —NHaryl, heteroaryl, or aryl;
A is CO or SO_{2;}
X is NR^{2′}, O, or S;
Y is N, CR^{2′} or if Y is O then R⁶ is absent;
Z is N or CR^{2′}; if Z is CH then X is O or NR^{2′}
R^{2′} is H, alkyl, —C(O)NR², —C(O)R^{b}, cycloalkyl, heterocycloalkyl, haloalkyl, hydroxyalkyl, hydroxyalkylamino, alkylamino, heteroaryl, or aryl;
n is 0 to 2;
p is 1 to 6;
q is 1 to 6;
R⁹ independently represents H, —CN, —OH, —SH, —CO₂R^{4′}, —C(O)R^{4a}, —C(O)NR⁷R⁸, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R⁴⁻, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, heterocycloalkyl, alkylamino, alkoxy, alkylthio, halogen, haloalkyl, haloalkyloxy, hydroxyalkylamino, hydroxyalkyl, hydroxycycloalkyl, aryl, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl, —(CH₂)ₚNR⁷COR⁸, or heteroaryl;
R^{4′}, R^{4″}, R^{5′} independently are H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, heteroaryl, or aryl;
R^{4a} is H, C₁-C₆-alkyl, C₂-C₆-alkenyl, cycloalkyl, haloalkyl, hydroxyalkyl, hydroxyalkylamino, alkylamino, —C(NR⁷)NR^{7′}R⁸, —(CH₂)ₚaryl, —CH₂)ₚNR⁷R⁸, —C(O)NR⁷R⁸, —N═CR⁷R⁸, —NR⁷C(O)R⁸, halogen, heteroaryl, or aryl

wherein

a C₁-C₆-alkyl group, if not stated otherwise, denotes a linear or branched C₁-C₆-alkyl, preferably a linear or branched chain of one to five carbon atoms, which can optionally be substituted by one or more substituents R′;

a C₂-C₆-alkenyl group, if not stated otherwise, denotes a linear or branched C₂-C₆-alkenyl, preferably a linear or branched chain of two to six carbon atoms, which can optionally be substituted by one or more substituents R′;

an alkyl group, if not stated otherwise, denotes a linear or branched C₁-C₆-alkyl, preferably a linear or branched chain of one to six carbon atoms, a linear or branched C₂-C₆-alkenyl or a linear or branched C₂-C₆-alkynyl group, which can be substituted by one or more substituents R′;

R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;

R″ is independently H, haloalkyl, hydroxyalkyl, alkyl, cycloalkyl, aryl, or heteroaryl;

a heterocycle denotes a heterocycloalkyl group or a heteroaryl group;

a cycloalkyl group denotes a non-aromatic ring system containing three to eight carbon atoms, preferably four to eight carbon atoms, wherein one or more of the carbon atoms in the ring can be substituted by a group R′ being as defined above; the C₃-C₈-cycloalkyl residue may be selected from the group comprising -cyclo-C₃H₅, -cyclo-C₄H₇, -cyclo-C₅H₉, -cyclo-C₆H₁₁, -cyclo-C₇H₁₃, -cyclo-C₈H₁₅;

a heterocycloalkyl group denotes a non-aromatic ring system containing two to ten carbon atoms and at least one heteroatom selected from O, N, and S, wherein one or more of the carbon atoms in the ring can be substituted by R′ being as defined above; preferred heterocycloalkyl groups are morpholine-4-yl, piperazinyl, 1-alkylpiperazine-4-yl, piperidinyl, pyrrolidinyl, azocane-1-yl;

an alkoxy group denotes an O-alkyl group, the alkyl group being as defined above; the alkoxy group is preferably a methoxy, ethoxy, isopropoxy, t-butoxy or pentoxy group;

an alkylthio group denotes an S-alkyl group, the alkyl group being as defined above;

a haloalkyl group denotes an alkyl group which is substituted by one to five halogen atoms, the alkyl group being as defined above; the haloalkyl group is preferably a —C(R¹⁰)₃, —CR¹⁰(R^{10′})₂, —CR¹⁰(R^{10′})R^{10″}, —C₂(R¹⁰)₅, —CH₂C(R¹⁰)₃, —CH₂CR¹⁰(R^{10′})₂, —CH₂CR¹⁰(R^{10′})R^{10″}, —C₃(R¹⁰)₇, or —C₂H₄C(R¹⁰)₃, wherein R¹⁰, R^{10′}, R^{10″} represent F, Cl, Br or I, preferably F;

a hydroxyalkyl group denotes an HO-alkyl group, the alkyl group being as defined above;

a haloalkyloxy group denotes an alkoxy group which is substituted by one to five halogen atoms, the alkyl group being as defined above; the haloalkyloxy group is preferably a —OC(R¹⁰)₃, —OCR¹⁰(R^{10′})₂, —OCR¹⁰(R^{10′})R^{10″}, —OC₂(R¹⁰)₅, —OCH₂C(R¹⁰)₃, —OCH₂CR¹⁰(R^{10′})₂, —OCH₂CR¹⁰(R^{10′})R^{10″}, —OC₃(R¹⁰)₇, or —OC₂H₄C(R¹⁰)₃, wherein R¹⁰, R^{10′}, R^{10″} represent F, Cl, Br or I, preferably F;

a hydroxyalkylamino group denotes an (HO-alkyl)₂-N— group or HO-alkyl-NH— group, the alkyl group being as defined above;

an alkylamino group denotes an HN-alkyl or N-dialkyl group, the alkyl group being as defined above;

a halogen group is fluorine, chlorine, bromine, or iodine;

an aryl group denotes an aromatic group having five to fifteen carbon atoms, which can be substituted by one or more substituents R′, where R′ is as defined above; the aryl group is preferably a benzyl group, a phenyl group, -o-C₆H₄—R′, -m-C₆H₄—R′, -p-C₆H₄—R′, 1-naphthyl, 2-naphthyl, 1-anthracenyl or 2-anthracenyl, R′ being as defined above;

a heteroaryl group denotes a 5- or 6-membered heterocyclic group which contains at least one heteroatom selected from O, N, and S. This heterocyclic group can be fused to another aromatic ring. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, benzooxazol-2-yl, benzooxazol-4-yl, benzooxazol-5-yl, benzoisooxazol-3-yl, benzoisooxazol-4-yl, benzoisooxazol-5-yl, 1,2,4-oxadiazol-3-yl, 1,2,4-oxadiazol-5-yl, 1,2,5-oxadiazol-3-yl, 1,2,5-oxadiazol-4-yl, 1,2,4-thiadiazol-3-yl, 1,2,4-thiadiazol-5-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, 6-pyrimidinyl, 2,4-dimethoxy-6-pyrimidinyl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1,3,5-triazol-6-yl, 2,4-dimethoxy-1,3,5-triazol-6-yl, 1H-tetrazol-2-yl, 1H-tetrazol-3-yl, tetrazolyl, acridyl, furazane, indazolyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazol-2-yl, 1H-benzimidazolyl, benzimidazol-4-yl, benzimidazol-5-yl, benzimidazol-6-yl, benzimidazol-7-yl, benzothiazolyl, quinazolinyl, quinoxazolinyl, cinnoline, quinolinyl, tetrahydroquinolinyl, isoquinolinyl, tetrahydro-thieno[3,4-d]imidazol-2-one, pyrazolo[5,1-c][1,2,4]triazine, 2,3-dihydrobenzo[1,4]-dioxin-2-yl, 2,3-dihydrobenzo[1,4]-dioxin-3-yl, 2,3-dihydrobenzo[1,4]-dioxin-5-yl, 2,3-dihydrobenzo[1,4]-dioxin-6-yl, 2,6-dimethoxypyrimidin-3-yl, 2,6-dimethoxypyrimidin-4-yl, tetrahydroisoquinolinyl, purine, phthalazine, pteridine, thiatetraazaindene, thiatriazaindene, isothiazolopyrazine, isothiazolopyrimidine, pyrazolotriazine, pyrazolopyrimidine, imidazopyridazine, imidazopyrimidine, imidazopyridine, imidazolotriazine, triazolotriazine, triazolopyridine, triazolopyrazine, triazolopyrimidine, 4-[1,2,4]triazolo[4,3-a]pyridin-3-yl, 1-furo[2,3-c]pyridin-4-yl, 1-furo[2,3-c]pyridin-5-yl, 1-furo[2,3-c]pyridin-3-yl, and triazolopyridazine group. This heterocyclic group can be substituted by one or more substituents R′, wherein R′ is as defined above.

In a preferred embodiment of the invention, in the compounds of formula (Ia), Z is S, Y is CO, X is CO, R is H, R^{c} is H, and R¹ is aryl, benzyl, or heteroaryl, R² is

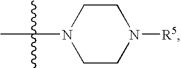

and R⁵ is optionally substituted aryl, benzyl or heteroaryl.

In a preferred embodiment of the invention, in the compounds of formula (Ia), Z is O, Y is CO, X is CO, R is H, R^{c} is H, and R¹ is aryl, benzyl, or heteroaryl, R² is

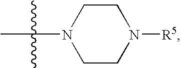

and R⁵ is optionally substituted aryl, benzyl or heteroaryl.

In a preferred embodiment of the invention, in the compounds of formula (Ib), Z is S, Y is CO, X forms a piperidine ring together with R¹ and R⁴, R^{a} and R^{b} is H, R^{c} is H or methyl, R² is

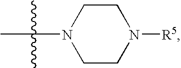

and R⁵ is optionally substituted aryl, benzyl or heteroaryl.

In a preferred embodiment of the invention, in the compounds of formula (Ib), Z is O, Y is CO, X forms a piperidine ring together with R¹ and R⁴, R^{a} and R^{b} is H, R^{c} is H or methyl, R² is

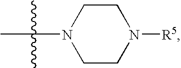

and R⁵ is optionally substituted aryl, benzyl or heteroaryl.

In another preferred embodiment, in the compounds of formula (Ic), r is 1, Y is CO, Z is O, t is 0, s is 1, X is CO, R^{c} is H, methyl, ethyl, methoxy, alkylamino, morpholino, N-methylpiperazine, CF₃, or OCF, R² is

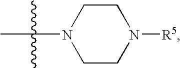

and R⁵ is optionally substituted aryl, benzyl or heteroaryl.

In another preferred embodiment, in the compounds of formula (Ic), r is 1, Y is CO, Z is S, t is 0, s is 0, R^{c} is H, methyl, ethyl, methoxy, alkylamino, morpholino, N-methylpiperazine, CF₃, or OCF, R² is

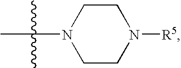

and R⁵ is optionally substituted aryl, benzyl or heteroaryl.

In another preferred embodiment, in the compounds of formula (Ic), r is 1, Y is CO, Z is S, t is 0, s is 1, X is CO, R^{c} is H, methyl, ethyl, methoxy, alkylamino, morpholino, N-methylpiperazine, CF₃, or OCF, R² is

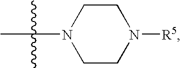

and R⁵ is optionally substituted aryl, benzyl or heteroaryl.

In another preferred embodiment, in the compounds of formula (Ic), r is 1, Y is CO, Z is O, t is 0, s is 0, R^{c} is H, methyl, ethyl, methoxy, alkylamino, morpholino, N-methylpiperazine, CF₃, or OCF, R² is

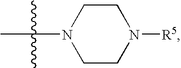

and R⁵ is optionally substituted aryl, benzyl or heteroaryl.

A preferred embodiment of the invention, in the compounds of formula (III), are compounds of the formula (IIIa),

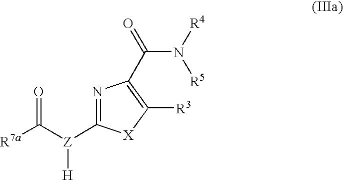

R³, R⁴, R⁵, R^{7a} and Z are defined as above.

Preferably, R³ is H, Me, OMe, CF₃, OCF₃, Cl, OH or SH, more preferably H, Me, OMe, CF₃, OCF₃, even more preferably H.

Preferably, Z is N.

Preferably, R⁴ is H.

Preferably, R^{7a} is aryl or heteroaryl, more preferably aryl, even more preferably phenyl. Further preferably, R^{7a} is substituted by one or two substituents R′, more preferably by one substituent R′. In a particularly preferred embodiment, R^{7a} is 2-trifluormethoxyphenyl. In another particularly preferred embodiment, R^{7a} is 2-methoxyphenyl. In another particularly preferred embodiment, R^{7a} is 2-chlorophenyl. And in yet another particularly preferred embodiment, R^{7a} is 2-methylphenyl.

Preferably, R⁵ is aryl or heteroaryl, more preferably heteroaryl. The heteroaryl contains preferably 2 nitrogen atoms and is particularly preferably 2-imidazolyl or 2-benzoimidazolyl, even more preferably 2-benzoimidazolyl. In another preferred embodiment, the heteroaryl contains 1 nitrogen atom and 1 sulfur atom and is particularly preferably 2-thiazolyl or 2-benzothiazolyl, even more preferably 2-benzothiazolyl. The heteroaryl further preferably is substituted by one to four substituents R′, more preferably by one or two substituents R′. In a particularly preferred embodiment, R⁵ is 5,6-dimethyl-1H-benzoimidazol-2-yl. In another particularly preferred embodiment, R⁵ is 5,6-dimethyl-1H-benzothiazol-2-yl.

Preferably, R′ is —SO₂-alkyl, —NO₂, halogen, —CO₂H, —OH, haloalkyloxy, alkoxy or alkyl. Particularly preferred for R′ are the following: methanesulfonyl, nitro, fluorine, chlorine, bromine, carboxyl group, hydroxyl, trifluormethoxy, methoxy, ethoxy, methyl, ethyl or trifluoroethyl.

In one preferred embodiment X is O. In another preferred embodiment X is S. In yet another preferred embodiment X is NR^{2′}.

In a preferred embodiment of the compound of formula (IIIa), R³ is H, methyl, methoxy, CF₃, or OCF₃; R⁴, R⁵, R^{7a} are defined as above; X is NR^{2′}, O or S; and Z is as defined as above.

Another preferred embodiment of the invention, in the compounds of formula (III), are compounds of the formula (IIIa), with R^{7a}═—NH-aryl.

Another preferred embodiment of the invention, in the compounds of formula (III), are compounds of the formula (IIIb),

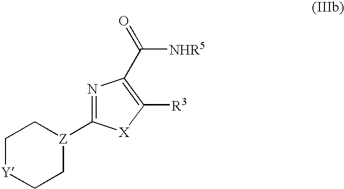

wherein

R³ is H, methyl, methoxy, CF₃, or OCF₃; R⁵ is defined as above; X is NR^{2′}, O or S; if Z is N then X is NR^{2′}, O or S; if Z is CR^{2′} then X is O; Y′ is O or NR^{2′}; R^{2′} is as defined above;

Another preferred embodiment of the invention, in the compounds of formula (III), are compounds of the formula (IIIc),

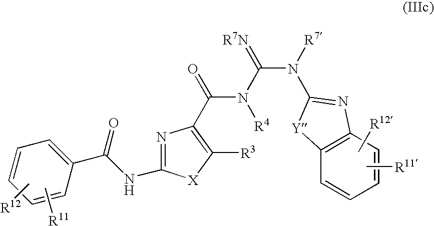

R¹¹, R¹² independently represent H, —CN, —OH, —SH, —CO₂R^{4′}, —C(O)R^{4′}, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, alkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl hydroxyalkyl, hydroxycycloalkyl, hydroxyalkylamino, halogen, haloalkyl, haloalkyloxy, aryl, arylalkyl or a heterocycle; R^{4′}, R^{4″}, R^{5′} are defined as above; R³ is H, methyl, methoxy, CF₃, or OCF₃; X is NR^{2′}, O or S; R⁴ is defined as above;

R⁷ and R^{7′} are defined as above; R^{11′}, R^{12′} independently represents H, —CN, —OH, —SH, —CO₂R^{4′}, —C(O)R^{4′}, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, alkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl hydroxyalkyl, hydroxycycloalkyl, hydroxyalkyl-amino, halogen, haloalkyl, haloalkyloxy, aryl, arylalkyl or a heterocycle; and R^{4′}, R^{4″}, R^{5′} are defined as above.

A more preferred embodiment of the invention, in the compounds of formula (III), are compounds of the formula (IIId),

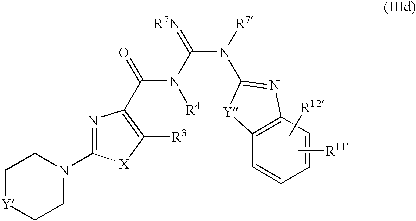

wherein

R³ is H, methyl, methoxy, CF₃, or OCF₃; X is NR^{2′}, O or S; R⁴ is defined as above; R⁷ and R^{7′} are defined as above; Y″ and R^{11′}, R^{12′} are defined as above under formula (IIIc); Y′ is O or NR^{2′} and R^{2′} is as defined above.

Another more preferred embodiment of the invention, in the compounds of formula (III), are compounds of the formula (IIIe),

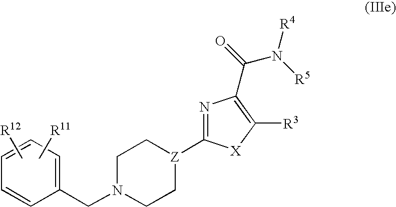

wherein

R³, R⁴ and R⁵ are defined as above; X is O or S; R¹¹ and R¹² are defined as above under formula (IIIc); and if Z is N, X is NR^{2′}, O or S; if Z is CH, X is O.

Another more preferred embodiment of the invention, in the compounds of formula (III), are compounds of the formula (IIIf),

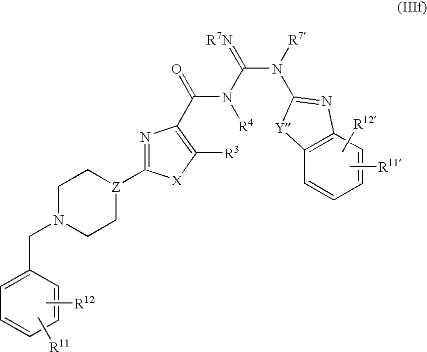

wherein

Z is N or CH; and R³ is H, methyl, methoxy, CF₃, or OCF₃; X is NR^{2′}, O or S; R⁴ is defined as above; R⁷ and R^{7′} are defined as above; R^{2′} is as defined above; Y″ and R¹¹, R^{11′}, R¹², R^{12′} are defined as above under formula (IIIc).

A preferred embodiment of the invention, are compounds of the formula (Iha),

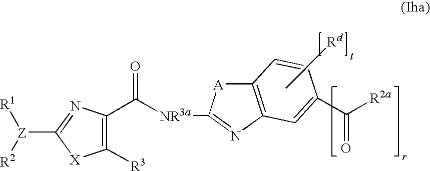

wherein

R¹ is COR^{7a};

R² and R^{3a} are H;

A is NH;

R⁷ is as defined above;

Z is N;

X is NR^{2′}, O or S;

R³ is H, methyl, ethyl, methoxy, amine, alkylamine, morpholino, N-methylpiperazine, CF₃, or OCF₃;

t is 0;

r is 1; and

R^{2a} is optionally substituted aryl, benzyl or heteroaryl.

Another preferred embodiment of the invention, in the compounds of formula (Ih), are compounds of the formula (Iha), wherein R^{7a} is optionally substituted aryl, benzyl or heteroaryl.

Another preferred embodiment of the invention, in the compounds of formula (Ih), are compounds of the formula (Ihb),

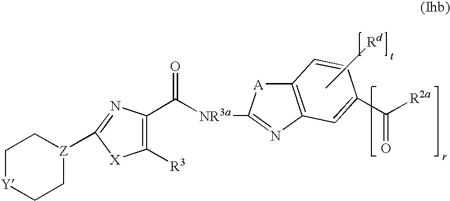

wherein

R^{3a} is H;

A is NH;

X is NR^{2′}, O or S;

Y′ is O or NR^{2′};

R^{2′} is as defined above;

R³ is H, methyl, ethyl, methoxy, amine, alkylamine, morpholino, N-methylpiperazine, CF₃, or OCF₃;

t is 0;

r is 1; and

R^{2a} is optionally substituted aryl, benzyl or heteroaryl.

In formula (Ihb), the following substituents are preferred, alone or in combination:

Z is preferably CR^{2′}.

Y′ is preferably NR²′. In a preferred embodiment, the R^{2′} in the NR^{2′} of Y′ is preferably substituted or unsubstituted heteroaryl. In another preferred embodiment, the R^{2′} in the NR^{2′} of Y′ is aryl. In yet another preferred embodiment, the R^{2′} in the NR^{2′} of Y′ is benzyl. In a more preferred embodiment, the R^{2′} in the NR^{2′} of Y′ is pyrimidine or triazine. In another more preferred embodiment, the R^{2′} in the NR^{2′} of Y′ is a substituted or unsubstituted bicyclic heteroaryl. more preferably thienopyrimidine, quinazoline, purine, pyrazolopyrimidine or triazolpyrimidine, even more preferably thienopyrimidine.

In a preferred embodiment, X is S. In another preferred embodiment, X is O. In yet another preferred embodiment, X is NR^{2′}.

R³ is preferably H.

R^{2a} is preferably aryl or heteroaryl, more preferably phenyl.

Another more preferred embodiment of the invention, in the compounds of formula (Ih), are compounds of the formula (Ihe),

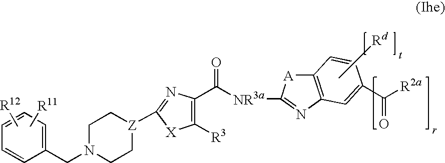

wherein

R^{3a} is H; A is NH; X is NR^{2′}, O or S;

R¹¹ and R¹² independently represent H, —CN, —OH, —SH, —CO₂R^{4′}, —C(O)R^{4′}, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, alkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′}benzthiazolyl, —C(NR^{4″})NR^{4′}benzoxazolyl hydroxyalkyl, hydroxycycloalkyl, hydroxyalkylamino, halogen, haloalkyl, haloalkyloxy, aryl, arylalkyl or a heterocycle;

R^{4′}, R^{4″}, R^{5′} are defined as above;

R^{2′} is as defined above;

R³ is H, methyl, ethyl, methoxy, amine, alkylamine, morpholino, N-methylpiperazine, CF₃, or OCF₃; t is 0; r is 1; and

R^{2a} is optionally substituted aryl, benzyl or heteroaryl.

In formula (Ihe), the following substituents are preferred, alone or in combination:

Z is preferably CR^{2′}.

In a preferred embodiment, X is S. In another preferred embodiment, X is O. In yet another preferred embodiment, X is NR^{2′}.

R¹¹ is preferably —H, —CN, —OH, halogen, haloalkyl, haloalkyloxy, more preferably —CN.

R¹² is preferably H.

R^{2a} is preferably aryl or heteroaryl, more preferably phenyl.

A preferred embodiment of the invention, in the compounds of formula (II), are compounds of the formula (IIa),

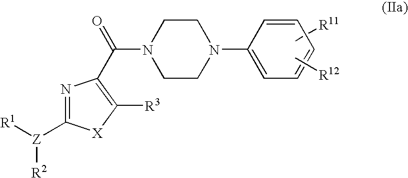

wherein

R¹ and R² are defined as above; Z is defined as above; X is O or S; R³ is H, methyl, methoxy, CF₃, or OCF₃; R¹¹ and R¹² are defined as above under formula (IIIc).

A more preferred embodiment of the invention, in the compounds of formula (II), are compounds of the formula (IIb),

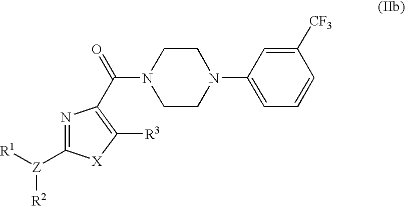

wherein

R¹ and R² are defined as above; Z is defined as above; X is O or S; R³ is H, methyl, methoxy, CF₃, or OCF₃.

Another more preferred embodiment of the invention, in the compounds of formula (II), are compounds of the formula (IIc),

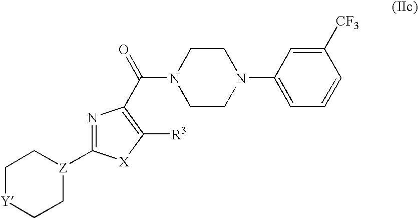

wherein

X is 0 or S; R³ is H, methyl, methoxy, CF₃, or OCF₃; Y′ is NR^{2′}.

Another more preferred embodiment of the invention, in the compounds of formula (II), are compounds of the formula (IId),

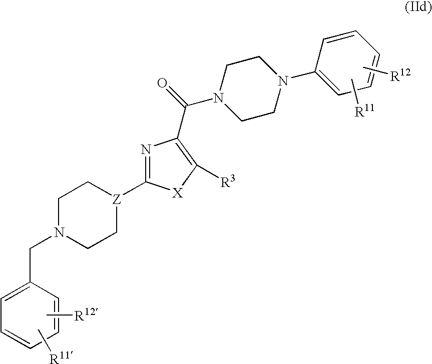

wherein

Z is defined as above; R³ is H, methyl, methoxy, CF₃, or OCF_{3;} R¹¹, R^{11′} and R¹², R^{12′} are defined as above.

In addition, the present invention provides methods for preparing the compounds of the invention such as compounds of formula (Ia), (Ib), (Ic), (Ih), (II), or (III).

The compounds of formula (Ia), (Ib), (Ic), (Ih), (II), or (III) may be obtained via various methods.

Piperidin-4yl-thiazole-4-carboxamide can be prepared by various methods described in the literature. One such example is the oxidation of the appropriate 2,5-dihydrothiazoles as described in Houben-Weyl, 2002, 730. The dihydrothiazoles can also be synthesised by methods described in the same reference or described in You, S., Razavi, H., Kelly, J. W. Angew. Chem. 2003, 115, 87 or Katritzky, A R., Cai, C., Suzuki, K., Singh, S K. J. Org. Chem. 2004, 69, 811-814 and references in both papers. Alternative methods were described by Yasuchika, S. et. al. Heterocycles, Vol. 57, No. 5, 2002.

The compounds of formula (II), (Ic), and (III) carrying a further substituent on the heterocycle can be obtained via various methods described in Organic Syntheses, Coll. Vol. 9, p. 155; and Vol. 74, p. 229; J. Org. Chem., 1975, Vol. 40, No. 10, page 1521, J. Am. Chem. Soc. 96:19/Sep. 18, 1974; J. Org. Chem., 1990, 55, 4484-4487; Chemische Berichte 1968, 101 (1), 302-307; Agricultural Chemistry and Biotechnology, 2002, 45 (1), page 37-42.

One possibility for the synthesis of substituted benzimindazole-substituent of formula (Ih) and (III) is described in Synthesis, 2006, 4, 597-602.

One possibility for the synthesis of compounds of formula (IIIa, c and IIa, b) (see scheme 1) comprises a step of reacting a compound of formula (V) with a compound of formula (VI) under classical amide coupling conditions, like e.g. HBTU, iPr₂NEt, DMF, 0° C. to r.t. to obtain intermediate (VII). Another alternative for this step might be the reaction of (V) with the corresponding acid chloride of (VI) to yield (VII). In a second step, compound (VII) is saponified with a 1 M NaOH solution, obtaining the expected acid (VIII) in almost quantitative yield. This step could be realized under acidic conditions as well. Finally, another amide coupling step (with primary or secondary amines), which works similarily to step 1 described above, completes the synthesis for compounds of type (IIIa, c and IIa, and b).

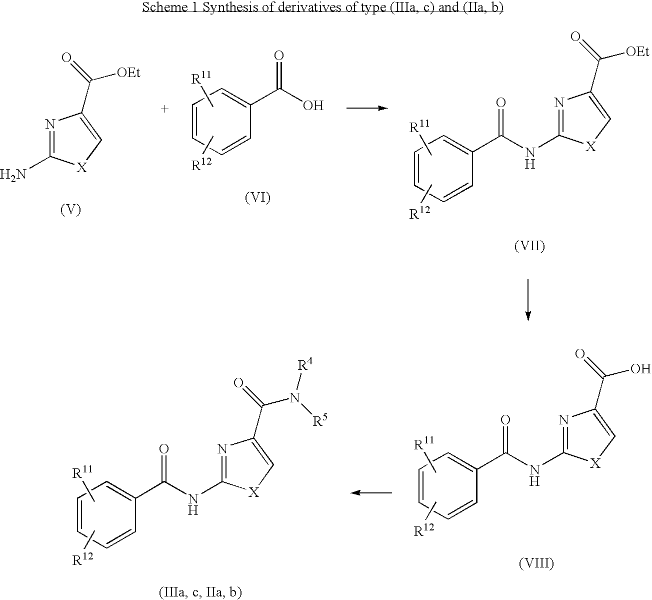

Compounds dealing with structure (IIIb, d, e, f) and (IIc, d) can be synthesized according to the procedure displayed in scheme 2. Herein, a heterocycle (IX) is reacted with a bromocompound (X), by means of a nucleophilic substitution reaction, to gain a bicyclic ester (XI), which is then saponified under standard and well-known conditions to acid (XII), completing the synthesis with another coupling step as described in scheme 1 above.

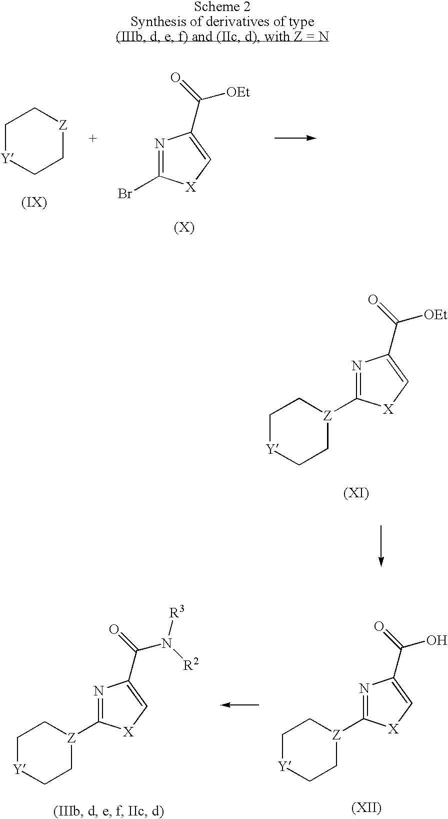

In case of Z═CH, structure (IIIb, d, e, f) and (IIc, d) type compounds can easily be synthesized following a protocol outlined in scheme 3, wherein a heterocycle (XIII) is converted to compound (XV) by a cyclokondensation step. After saponification, (XVI) is coupled with an amine to yield the desired product (IIIb, d, e, f) or (IIc, d).

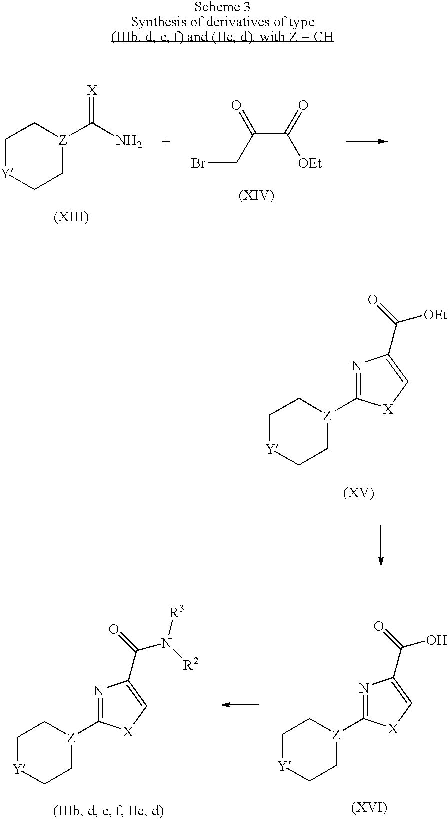

Compounds of the present invention carrying a piperidin-4-yl substituent in the 2-position of the thiazole ring can, for example, be prepared as shown in the following scheme. This synthetic route is partially described in WO 2004/058750.

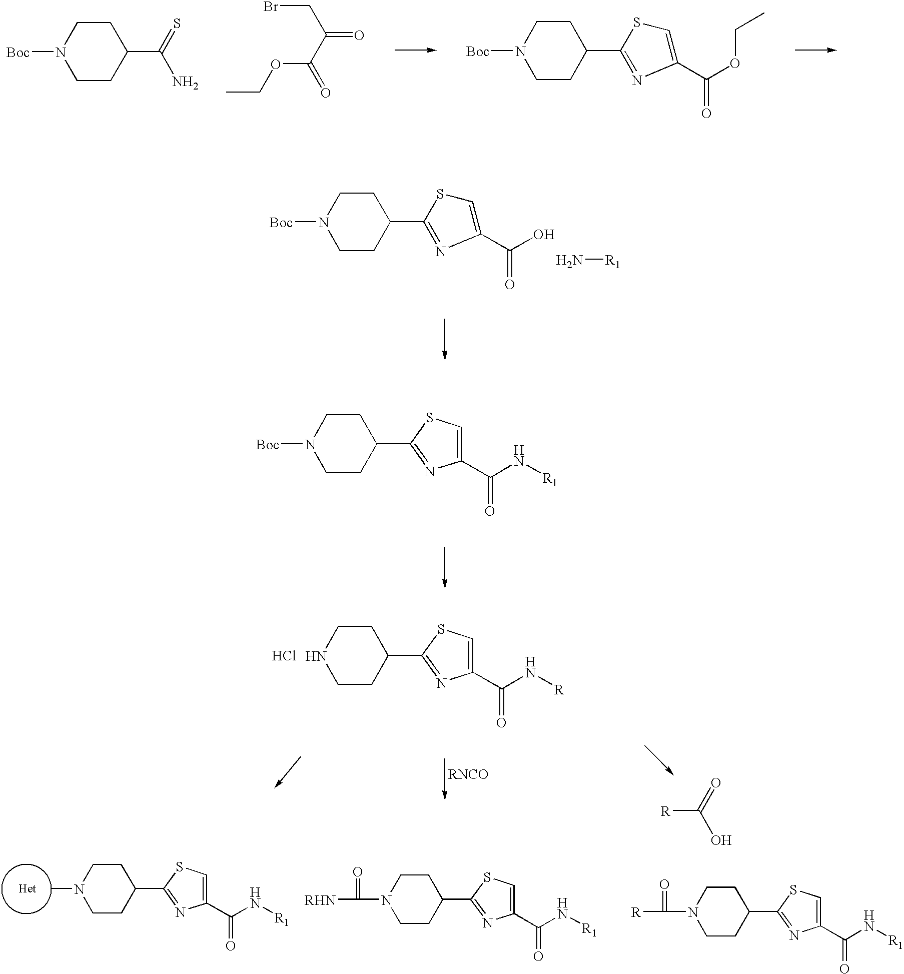

2-(1-(tert-butoxycarbonyl)piperidin-4-yl)thiazole-4-carboxylic acid can be converted to the appropriate R¹ amide by coupling with HBTU, DIPEA in DMF. The different R¹-amines are either commercially available or can be readily synthesized. The Boc-protection group can be removed under standard conditions, such as treatment with TFA for 2 to 3 hours at 0° or with 4 N HCl in dioxane for 2 to 3 hours. The deprotected piperidinoderivative HCl salt can then be converted to the corresponding amides, ureas and N-heterocyclic analogs as follows.

Urea substituted piperidino compounds can be synthesized by coupling with commercially available isocyanates in the presence of DIPEA. Piperidino compounds substituted with heterocycles can be synthesized by standard procedures, such as coupling with the corresponding chloroheterocycle in the presence of a base. Alternatively, piperidino compounds substituted with heterocycles can be obtained by palladium-mediated cross coupling. As a further alternative, hydroxypyridine derivatives can be coupled to the piperidino compound by the HBTU coupling method.

An alternative route to compounds of the present invention carrying a piperidin-4-yl substituent in the 2-position of the thiazole ring is shown in the following scheme.

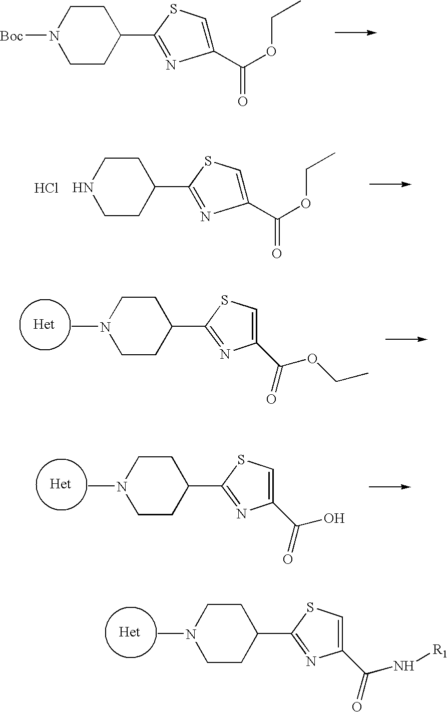

The Boc-protection group can be removed under standard conditions, such as treatment with TFA for 2 to 3 hours at 0° or with 4 N HCl in dioxane for 2 to 3 hours. The deprotected piperidinoderivative HCl salt can then be converted to the corresponding N-heterocyclic analog by various methods as described as described above.

For the compounds of the formula (I), (Ia), (Ib), (Ic), (Ih), (II), or (III) above, the term “stereoisomer” means cis/trans or E/Z isomerism. More particularly, the possible double bond(s) present in the various substituent of the compounds of the present invention can be E or Z configuration. These pure or impure geometrical isomers, alone or as a mixture, form an integral part of the compounds of the formula (I), (Ia), (Ib), (Ic), (Ih), (II), or (III). The term “stereoisomer” includes also all the isomeric forms, alone or as mixture, resulting from the presence of one or more axes and/or centres of symmetry in the molecules, and resulting in the rotation of a beam of polarized light. More particularly, it includes enatiomers and diastereomers, in pure form or as a mixture.

The compounds of the formula (I), (Ia), (Ib), (Ic), (Ih), (II), or (III) to be used according to the invention can form salts with inorganic or organic acids or bases. Examples of pharmaceutically acceptable salts comprise without limitation non-toxic inorganic or organic salts such as acetate derived from acetic acid, aconitate derived from aconitic acid, ascorbate derived from ascorbic acid, benzoate derived from benzoic acid, cinnamate derived from cinnamic acid, citrate derived from citric acid, embonate derived from embonic acid, enantate derived from heptanoic acid, formiate derived from formic acid, fumarate derived from fumaric acid, glutamate derived from glutamic acid, glycolate derived from glycolic acid, chloride derived from hydrochloric acid, bromide derived from hydrobromic acid, lactate derived from lactic acid, maleate derived from maleic acid, malonate derived from malonic acid, mandelate derived from mandelic acid, methanesulfonate derived from methanesulfonic acid, naphtaline-2-sulfonate derived from naphtaline-2-sulfonic acid, nitrate derived from nitric acid, perchlorate derived from perchloric acid, phosphate derived from phosphoric acid, phthalate derived from phthalic acid, salicylate derived from salicylic acid, sorbate derived from sorbic acid, stearate derived from stearic acid, succinate derived from succinic acid, sulphate derived from sulphuric acid, tartrate derived from tartaric acid, toluene-p-sulfate derived from p-toluene-sulfonic acid and others. Such salts can be produced by methods known to someone of skill in the art and described in the prior art.

Other salts like oxalate derived from oxalic acid, which is not considered as pharmaceutically acceptable can be appropriate as intermediates for the production of compounds of the formula (I), (Ia), (Ib), (Ic), (Ih), (II), or (III) or a pharmaceutically acceptable salt thereof or pharmaceutically acceptable prodrugs, or a stereoisomer thereof.

The invention covers the pharmaceutically acceptable salts, as indicated above, but also salts allowing a suitable seperation or crystallization of the compounds of the formula (I), (Ia), (Ib), (Ic), (Ih), (II), or (III), such as the salts obtained with chiral amines.

The compounds of the formula (I), (Ia), (Ib), (Ic), (Ih), (II), or (III) above also comprise the prodrugs of these compounds.

The term “prodrug” as used herein refers to compounds which once administered to the patient are not pharmaceutically active themselves (‘prodrugs’) but which are chemically and/or biologically transformed into their pharmaceutical active form (compounds of formula (I), (Ia), (Ib), (Ic), (Ih), (II), or (III)) in vivo, i.e. in the subject to which the compound is administered. Prodrugs include, for example, compounds of the invention wherein hydroxy, amine or sulfhydryl groups are bonded to any group that, when administered to a patient, cleaves to form the hydroxy, amine or sulfhydryl groups. Thus, representative examples of prodrugs include, but are not limited to, acetate, formate and benzoate derivatives of alcohol, sulfhydryl and amine functional groups of the compounds of the present invention. Further, in the case of carboxylic acid (—COOH), esters may be employed, such as methyl esters, ethyl esters, double esters and the like. Esters may be active in their own right and/or be hydrolysable under in vivo conditions in the human body.

The compounds according to the invention and medicaments prepared therewith are generally useful for the treatment of cell proliferation disorders, for the treatment or prophylaxis of immunological diseases and conditions (as for instance inflammatory diseases, neuroimmunological diseases, autoimmune diseases or other).

The compounds of the present invention are useful for the treatment of diseases which are caused by malignant cell proliferation, such as all forms of solid tumors, leukemias and lymphomas. Therefore the compounds according to the invention and medicaments prepared therewith are generally useful for regulating cell activation, cell proliferation, cell survival, cell differentiation, cell cycle, cell maturation and cell death or to induce systemic changes in metabolism such as changes in sugar, lipid or protein metabolism. They can also be used to support cell generation poiesis, including blood cell growth and generation (prohematopoietic effect) after depletion or destruction of cells, as caused by, for example, toxic agents, radiation, immunotherapy, growth defects, malnutrition, malabsorption, immune dysregulation, anemia and the like or to provide a therapeutic control of tissue generation and degradation, and therapeutic modification of cell and tissue maintenance and blood cell homeostasis.

These diseases and conditions include but are not limited to cancer as hematological (e.g. leukemia, lymphoma, myeloma) or solid tumors (for example breast, prostate, liver, bladder, lung, esophageal, stomach, colorectal, genitourinary, gastrointestinal, skin, pancreatic, brain, uterine, colon, head and neck, cervical, and ovarian, melanoma, astrocytoma, small cell lung cancer, glioma, basal and squameous cell carcinoma, sarcomas as Kaposi's sarcoma and osteosarcoma) or for the treatment of diseases which are cured or relieved by the inhibition of one or several kinases and/or phosphatases. The compounds according to the invention and medicaments prepared therewith are particularly useful for the treatment of prostate cancer, melanoma, ovarial cancer and multiple myeloma.

“Treatment” according to the present invention is intended to mean complete or partial healing of a disease, or alleviation of a disease or stop of progression of a given disease.

Thus, in one embodiment, the invention relates to the use of the compounds of the formula (I), (Ia), (Ib), (Ic), (Ih), (II), or (III) or a pharmaceutically acceptable salt or pharmaceutically acceptable prodrugs, or a stereoisomer thereof if desired with appropriate adjuvants and additives for the production of a medicament for the treatment or prevention of a disease characterized by hyperproliferation of keratinocytes and/or T cells, especially inflammatory disorders and immune disorders, preferably selected from the group consisting of Addison's disease, alopecia areata, Ankylosing spondylitis, haemolytic anemia (anemia haemolytica), pernicious anemia (anemia pemiciosa), aphthae, aphthous stomatitis, arthritis, arteriosclerotic disorders, osteoarthritis, rheumatoid arthritis, aspermiogenese, asthma bronchiale, auto-immune asthma, auto-immune hemolysis, Bechet's disease, Boeck's disease, inflammatory bowel disease, Burkitt's lymphoma, Crohn's disease, chorioiditis, colitis ulcerosa, Coeliac disease, cryoglobulinemia, dermatitis herpetiformis, dermatomyositis, insulin-dependent type I diabetes, juvenile diabetes, idiopathic diabetes insipidus, insulin-dependent diabetes mellisis, autoimmune demyelinating diseases, Dupuytren's contracture, encephalomyelitis, encephalomyelitis allergica, endophthalmia phacoanaphylactica, enteritis allergica, autoimmune enteropathy syndrome, erythema nodosum leprosum, idiopathic facial paralysis, chronic fatigue syndrome, febris rheumatica, glomerulo nephritis, Goodpasture's syndrome, Graves' disease, Harnman-Rich's disease, Hashimoto's disease, Hashimoto's thyroiditis, sudden hearing loss, sensoneural hearing loss, hepatitis chronica, Hodgkin's disease, haemoglobinuria paroxysmatica, hypogonadism, ileitis regionalis, iritis, leucopenia, leucemia, lupus erythematosus disseminatus, systemic lupus erythematosus, cutaneous lupus erythematosus, lymphogranuloma malignum, mononucleosis infectiosa, myasthenia gravis, traverse myelitis, primary idiopathic myxedema, nephrosis, ophthalmia symphatica, orchitis granulomatosa, pancreatitis, pemphigus, pemphigus vulgaris, polyarteritis nodosa, polyarthritis chronica primaria, polymyositis, polyradiculitis acuta, psoriasis, purpura, pyoderma gangrenosum, Quervain's thyreoiditis, Reiter's syndrome, sarcoidosis, ataxic sclerosis, progressive systemic sclerosis, scleritis, sclerodermia, multiple sclerosis, sclerosis disseminata, acquired spenic atrophy, infertility due to antispermatozoan antibodies, thrombocytopenia, idiopathic thrombocytopenia purpura, thymoma, acute anterior uveitis, vitiligo, AIDS, HIV, SCID and Epstein Barr virus associated diseases such as Sjorgren's syndrome, virus (AIDS or EBV) associated B cell lymphoma, parasitic diseases such as Leishmania, and immunesuppressed disease states such as viral infections following allograft transplantations, AIDS, cancer, chronic active hepatitis diabetes, toxic chock syndrome and food poisoning. The compounds according to the invention and medicaments prepared therewith are particularly useful for the treatment of autoimmune diseases and inflammatory diseases, such as rheumatoid arthritis, multiple sclerosis, inflammatory bowel disease (in particular colitis and morbus crohn), inflammatory skin diseases (in particular neurodermitis and psoriasis) and lupus erythematosus.

Furthermore, the invention relates to a method of treatment or prevention of diseases which comprises the administration of an effective amount of compounds of the (I), (Ia), (Ib), (Ic), (Ih), (II), or (III) or a pharmaceutically acceptable salt or pharmaceutically acceptable prodrugs,or a stereoisomer thereof.

The invention also provides a pharmaceutical composition comprising a compound of formula (I), (Ia), (Ib), (Ic), (Ih), (II), or (III), in free form or in the form of pharmaceutically acceptable salts and pharmaceutically acceptable prodrugs, together with a pharmaceutically acceptable diluent or carrier therefore.

In a preferred embodiment, the invention relates to the use of compounds of the formula (I), (Ia), (Ib), (Ic), (Ih), (II), or (III), or a pharmaceutically acceptable salt or pharmaceutically acceptable prodrugs or a stereoisomer thereof if desired with appropriate adjuvants and additives for the production of a medicament for the treatment or prevention of skin diseases in which T cells play a role; especially preferably the skin diseases are selected from the group consisting of psoriasis, atopic dermatitis, alopecia areata, alopecia totalis, alopecia subtotalis, alopecia universalis, alopecia diffusa, lupus erythematodes of the skin, lichen planus, dermatomyostis of the skin, atopic eczema, morphea, sklerodermia, psoriasis vulgaris, psoriasis capitis, psoriasis guttata, psoriasis inversa, alopecia areata ophiasis-type, androgenetic alopecia, allergic contact eczema, irritative contact eczema, contact eczema, pemphigus vulgaris, pemphigus foliaceus, pemphigus vegetans, scarring mucosal pemphigoid, bullous pemphgoid, mucous pemphigoid, dermatitis, dermatitis herpetiformis duhring, urticaria, necrobiosis lipoidica, erythema nodosum, lichen vidal, prurigo simplex, prurigo nodularis, prurigo acuta, linear IgA dermatosis, polymorphic light dermatoses, erythema solaris, lichen sclerosus et atrophicans, exanthema of the skin, drug exanthema, purpura chronica progressiva, dihidrotic eczema, Eczema, fixed drug exanthema, photoallergic skin reaction, lichen simplex eriorale, dermatitis and “Graft versus Host-Disease”, acne, rosacea, scarring, keloids and vitiligo.

Moreover, the compounds of the present invention can be used for the treatment of diseases resulting from ischemia and/or reperfusion injury of organs and/or of parts of the body selected from the group comprising heart, brain, peripheral limb, kidney, liver, spleen and lung, and/or wherein the endothelial dysfunction is associated with diseases selected from a group comprising infarctions such as myocardial infarction and critical limb ischemia, and/or wherein the endothelial dysfunction is associated with diseases selected from the group comprising ischemic diseases such as peripheral arterial occlusive disease, e.g. critical leg ischemia, myocardial infarction and ischemic diseases of organs, e. g. of the kidney, spleen, brain and lung.

The compounds of this invention also can be applied for the prevention and the treatment of neurological diseases or disorders (diseases or disorders associated with the brain and nervous system), including but not limited to, Alzheimer's disease, Parkinson's disease, Creutzfeld-Jacob Disease, Lewy Body Dementia, amyotrophic lateral sclerosis, stroke, epilepsy, multiple sclerosis, myasthenia gravis, Huntington's Disease, Down's Syndrome, nerve deafness, and Meniere's disease.). Other neurological diseases and disorders will be apparent to those of skill in the art and are encompassed by the definition as used in this invention. The compounds according to the invention and medicaments prepared therewith are particularly useful for the treatment of stroke, reperfusion injury and Alzheimer's disease.

The compounds of the present invention can further be used for diseases that are caused by protozoal infestations in humans and animals. Such veterinary and human pathogenic protozoal are preferably intracellular active parasites of the phylum Apicomplexa or Sarcomastigophora, especially Trypanosoma, Plasmodia, Leishmania, Babesia and Theileria, Cryptosporidia, Sacrocystida, Amoebia, Coccidia and Trichomonadia. These active substances or corresponding drugs are especially suitable for the treatment of Malaria tropica, caused by Plasmodium falciparum, Malaria tertiana, caused by Plasmodium vivax or Plasmodium ovale and for the treatment of Malaria quartana, caused by Plasmodium malariae. They are also suitable for the treatment of Toxoplasmosis, caused by Toxoplasma gondii, Coccidiosis, caused for instance by Isospora belli, intestinal Sarcosporidiosis, caused by Sarcocystis suihominis, dysentery caused by Entamoeba histolytica, Cryptosporidiosis, caused by Cryptosporidium parvum, Chargas' disease, caused by Trypanosoma cruzi, sleeping sickness, caused by Trypanosoma brucei rhodesiense or gambiense, the cutaneous and visceral as well as other forms of Leishmaniosis. They are also suitable for the treatment of animals infected by veterinary pathogenic protozoa, like Theileria parva, the pathogen causing bovine East coast fever, Trypanosoma congolense congolense or Trypanosoma vivax vivax, Trypanosoma brucei brucei, pathogens causing Nagana cattle disease in Africa, Trypanosoma brucei evansi causing Surra, Babesia bigemina, the pathogen causing Texas fever in cattle and buffalos, Babesia bovis, the pathogen causing European bovine Babesiosis as well as Babesiosis in dogs, cats and sheep, Sarcocystis ovicanis and ovifelis pathogens causing Sarcocystiosis in sheep, cattle and pigs, Cryptosporidia, pathogens causing Cryptosporidioses in cattle and birds, Eimeria and Isospora species, pathogens causing Coccidiosis in rabbits, cattle, sheep, goats, pigs and birds, especially in chickens and turkeys. The use of the compounds of the present invention is preferred in particular for the treatment of Coccidiosis or Malaria infections, or for the preparation of a drug or feed stuff for the treatment of these diseases. This treatment can be prophylactic or curative. In the treatment of malaria, the compounds of the present invention may be combined with other anti-malaria agents.

The compounds of the present invention can further be used for the prophylaxis and/or treatment of infectious diseases caused among others by bacteria and viruses, including opportunistic infections in a mammal, including a human. Said method comprises administering to the mammal an amount of at least one compound of the general formula (I), (Ia), (Ib), (Ic), (Ih), (II), or (III) and/or pharmaceutically acceptable salts thereof, effective to prevent and/or treat said infectious disease and/or opportunistic infection.

The infectious disease can be selected from the group comprising AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection (CMV), Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, diseases caused by staphylococcal infections (Staphylococcus aureus and other staphylococcus species), diseases caused by infections with pseudomonas aeruginosa and other pseudomonas species, Burkholderia cepacia infections, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis, Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever. The compounds according to the invention and medicaments prepared therewith are particularly useful for the treatment of viral diseases, such as hepatits B, hepatitis C, influenza virus infections (in particular influenza A virus infections), AIDS (HIV infections) and human papilloma virus infections.

The compounds according to the invention and medicaments prepared therewith are also particularly useful for the treatment of artheriosclerosis.

The compounds according to the invention and medicaments prepared therewith are further particularly useful for the treatment of osteoporosis.

In one embodiment, the present invention relates to the use of a compound or a composition of the present invention for the preparation of a medicament for the treatment or prevention of a disease characterized by hyperproliferation of cells.

In one embodiment, the present invention relates to the use of a compound or a composition of the present invention for the preparation of a medicament for the treatment or prevention of a disease resulting from ischemia and/or reperfusion injury of organs and/or of parts of the body selected from the group comprising heart, brain, peripheral limb, kidney, liver, spleen and lung, and/or wherein the endothelial dysfunction is associated with diseases selected from a group comprising infarctions such as myocardial infarction and critical limb ischemia, and/or wherein the endothelial dysfunction is associated with diseases selected from the group comprising ischemic diseases, myocardial infarction and ischemic diseases of organs.

In one embodiment, the present invention relates to the use of a compound or a composition of the present invention for the preparation of a medicament for the treatment or prevention of a neurological diseases or disorders selected from the group comprising Alzheimer's disease, Parkinson's disease, Creutzfeld-Jacob Disease, Lewy Body Dementia, amyotrophic lateral sclerosis, stroke, epilepsy, multiple sclerosis, myasthenia gravis, Huntington's Disease, Down's Syndrome, nerve deafness, and Meniere's disease.

The compounds of formula (I), (Ia), (Ib), (Ic), (Ih), (II), or (III) and their pharmacologically acceptable salts can be administered to animals, preferably to mammals, and in particular to humans, dogs and chickens as therapeutics per se, as mixtures with one another or in the form of pharmaceutical preparations which allow enteral or parenteral use and which as active constituent contain an effective dose of at least one compound of the formula (I), (Ia), (Ib), (Ic), (Ih), (II), or (III) or a salt thereof, in addition to customary pharmaceutically innocuous excipients and additives. The compounds of formula (I), (Ia), (Ib), (Ic), (Ih), (II), or (III) can also be administered in form of their salts, which are obtainable by reacting the respective compounds with physiologically acceptable acids and bases.

The production of medicaments containing the compounds of formula (I), (Ia), (Ib), (Ic), (Ih), (II), or (III) according to the invention and their application can be performed according to well-known pharmaceutical methods.

While the compounds of formula (I), (Ia), (Ib), (Ic), (Ih), (II), or (III) according to the invention for use in therapy may be administered in the form of the raw chemical compound, it is preferred to introduce the active ingredient, optionally in the form of a physiologically acceptable salt in a pharmaceutical composition together with one or more adjuvants, excipients, carriers, buffers, diluents, and/or other customary pharmaceutical auxiliaries. Such salts of the compounds may be anhydrous or solvated.

In a preferred embodiment, the invention provides medicaments comprising compounds of formula (I), (Ia), (Ib), (Ic), (Ih), (II), or (III) according to the invention, or a pharmaceutically acceptable salt or pharmaceutically acceptable prodrugs or a stereoisomer thereof, together with one or more pharmaceutically acceptable carriers thereof, and, optionally, other therapeutic and/or prophylactic ingredients. The carrier(s) must be “acceptable” in the sense of being compatible with the other ingredients of the formulation and not harmful to the recipient thereof.

A medicament of the invention may be those suitable for oral, rectal, bronchial, nasal, topical, buccal, sub-lingual, transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The compounds according to the invention, together with a conventional adjuvant, carrier, or diluent, may thus be placed into the form of medicament and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such Medicament and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The compound useable according to the invention can be administered in a wide variety of oral and parenteral dosage forms. It will be obvious to those skilled in the art that the following dosage forms may comprise, as the active component, either a compound of formula (I), (Ia), (Ib), (Ic), (Ih), (II), or (III) according to the invention or a pharmaceutically acceptable salt or stereosomer thereof.

For preparing a medicament from a compounds of formula (I), (Ia), (Ib), (Ic), (Ih), (II), or (III) pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify. Compositions suitable for vaginal administration may be presented as pessaries, tampons, creams, gels, pastes, foams or sprays containing in addition to the active ingredient such carriers as are known in the art to be appropriate. Liquid preparations include solutions, suspensions, and emulsions, for example, water or water-propylene glycol solutions. For example, parenteral injection liquid preparations can be formulated as solutions in aqueous polyethylene glycol solution.

The compounds of formula (I), (Ia), (Ib), (Ic), (Ih), (II), or (III) according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilizing agents, and the like.

In one embodiment of the present invention, the medicament is applied topically or systemically or via a combination of the two routes.

In an especially preferred embodiment of the present invention the medicament is applied topically. This reduces possible side effects and limits the necessary treatment to those areas affected.

Preferably the medicament is prepared in form of an ointment, a gel, a plaster, an emulsion, a lotion, a foam, a cream of a mixed phase or amphiphilic emulsion system (oil/water-water/oil mixed phase), a liposome, a transfersome, a paste or a powder.

Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form. In the latter case of a dropper or pipette, this may be achieved by the patient administering an appropriate, predetermined volume of the solution or suspension.

In the case of a spray, this may be achieved for example by means of a metering atomising spray pump.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form. Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co. Easton, Pa.).

Pharmaceutical compositions can also contain two or more compounds of the formula (I), (Ia), (Ib), (Ic), (Ih), (II), or (III) or their pharmacologically acceptable salts and also other therapeutically active substances.

Thus, the compounds of the present invention can be used in the form of one compound alone or in combination with other active compounds—for example with medicaments already known for the treatment of the aforementioned diseases, whereby in the latter case a favorable additive, amplifying effect is noticed. Suitable amounts to be administered to humans may range from 5 to 500 mg.

To prepare the pharmaceutical preparations, pharmaceutically inert inorganic or organic excipients can be used. To prepare pills, tablets, coated tablets and hard gelatin capsules, for example, lactose, corn starch or derivatives thereof, talc, stearic acid or its salts, etc. can be used. Excipients for soft gelatin capsules and suppositories are, for example, fats, waxes, semi-solid and liquid polyols, natural or hardened oils etc. Suitable excipients for the production of solutions and syrups are, for example, water, sucrose, invert sugar, glucose, polyols etc. Suitable excipients for the production of injection solutions are, for example, water, alcohols, glycerol, polyols or vegetable oils.

The dose can vary within wide limits and is to be suited to the individual conditions in each individual case. For the above uses the appropriate dosage will vary depending on the mode of administration, the particular condition to be treated and the effect desired. In general, however, satisfactory results are achieved at dosage rates of about 1 to 100 mg/kg animal body weight preferably 1 to 50 mg/kg. In general, suitable dosage rates for larger mammals, for example humans, may be of the order of from about 10 mg to 3 g/day, conveniently administered once, in divided doses 2 to 4 times a day, or in sustained release form.

In general, a daily dose of approximately 10 mg to 5000 mg, preferably 50 to 500 mg, per human individual is appropriate in the case of the oral administration. In the case of other administration forms too, the daily dose is in similar ranges. For topical delivery, depending on the permeability of the skin, the type and the severity of the disease and dependent on the type of formulation and frequency of application, different concentrations of active compounds within the medicament can be sufficient to elicit a therapeutic effect by topical application. Preferably the concentration of an active compound or a pharmaceutically acceptable salt thereof or a physiologically functional derivative or a stereoisomer thereof within a medicament according to the invention is in the range of between 1 μmol/l and 100 mmol/l.

The following examples and figures are included to demonstrate preferred embodiments of the invention. It should be appreciated by those of skill in the art that the techniques disclosed in the examples that follow represent techniques discovered by the inventors to function well in the practice of the invention, and thus can be considered preferred modes for its practice. However, those of skill in the art should, in light of the present disclosure, appreciate that many changes can be made in the specific embodiments that are disclosed without departing from the spirit and scope of the invention as set out in the appended claims. All references cited are incorporated herein by reference.

### Examples

Abbreviations: min, minute(s); h, hour(s); r.t., room temperature; t-, tert-.

NMR spectra: Bruker Avance 300 MHz. The spectra were recorded at 300 MHz (¹H-NMR), respectively, using the residual solvent peak as an internal standard (DMSO-d₆, δ_{H}=2.49; CD₃OD, δ_{H}=3.31; CDCl₃, δ_{H}=7.26; CD₃CN, δ_{H}=1.93; (CD₃)₂CO, δ_{H}=2.05).

Analytical LC/ESI-MS: 2× Waters 600 Multisolvent Delivery System. 50 μl sample loop. Column, Chromolith Speed ROD RP18e (Merck, Darmstadt), 50×4.6 mm, with 2 μm prefilter (Merck). Eluent A, H₂O+0.1% HCO₂H; eluent B, MeCN. Gradient, 5% B to 100% B within 5 min; flow, 3 ml/min Waters LCZ single quadrupol mass spectrometer with electrospray source. MS method, MS8 minPM-80-800-20V; positive/negative ion mode scanning, m/z 80-800 in 1 s; capillary, 3.5 kV; cone voltage, 20 V; multiplier voltage, 400 V; probe and desolvation gas temperature, 120° C. and 350° C., respectively. Waters 2487 Dual λ Absorbance Detector, set to 254 nm.

Preparative HPLC-MS: Waters 600 Multisolvent Delivery System with peparative pump heads. 2000 μl or 5000 μl sample loop. Column, Waters X-Terra RP18, 7 μm, 19×150 mm with X-Terra RP18 guard cartridge 7 μm, 19×10 mm; used at flow rate 20 ml/min or YMC ODS-A, 120 Å, 40×150 mm with X-Terra RP18 guard cartridge 7 μm, 19×10 mm; used at flow rate 50 ml/min. Make-up solvent: MeCN—H₂O—HCO₂H 80:20:0.05 (v:v:v). Eluent A, H₂O+0.1% HCO₂H; eluent B, MeCN. Different linear gradients from 5-100% eluent B, adapted to sample. Injection volume: 500 μl-2000 μl depending on sample. Waters ZQ single quadrupol mass spectrometer with electrospray source. Positive or negative ion mode scanning m/z 80-800 in 1 s; capillary, 3.5 kV or 3.0 kV; cone voltage, 20 V; multiplier voltage, 400 V; probe and desolvation gas temperature, 120° C. and 350° C., respectively. Waters Fraction Collector II with mass-triggered fraction collection. Waters 996 photo diode array detector.

### Synthesis of 4-(Methoxy-methyl-carbamoyl)-piperidine-1-carboxylic acid t-butyl ester

Piperidine-1,4-dicarboxylic acid mono-tert-butyl ester (1.0 eq, 21.8 mmol) was dissolved under inert conditions in 35 ml dry N,N-dimethylformamide. O,N-dimethyl-hydroxylamine hydrochloride (1.03 eq, 22.5 mmol), benzotriazol-1-ol monohydrate (1.03 eq, 22.5 mmol) and triethylamine (1.5 eq, 32.7 mmol) were added. The reaction mixture was cooled to 0° C., N-(3-Dimethylaminopropyl)-N-ethylcarbodiimid hydrochloride (1.0 eq, 21.8 mmol) was added over a period of 10 minutes and the mixture was stirred vigorously at 0° C. for 1 h and at r.t. for 18 h.

The solvent was removed under vaccum and the residue was suspended in 400 ml ethylacetate. The organic layer was extracted 3 times with 100 ml of 1 M citric acid, aqueous sodium carbonate and twice with 100 ml brine, dried over MgSO₄ and filtered. The solvent was removed and the residue was purified by distillation resulting in a yield of 80%.

### Synthesis of 4-Formyl-piperidine-1-carboxylic acid t-butyl ester

4-(methoxy-methyl-carbamoyl)-piperidine-1-carboxylic acid tert-butyl ester (1.0 eq, 16.4 mmol) was dissolved in 100 ml dry tetrahydrofurane under inert atmosphere. This solution was added dropwise over a period of 1 h to a suspension of lithiumalanate (3.0 eq, 49.6 mmol) in 70 ml dry tetrahydrofurane at −50° C. During the adding of the mixture, the temperature was held at −50° C. and then allowed to warm to 0° C. within 3 h.

The mixture was cooled to −78° C. and quenched carefully with 100 ml 1 M citric acid. The mixture was warmed up to r.t. and diluted with 400 ml ethylacetate. The phases were separated and the aqueous phase was extracted 3 times with 70 ml ethylacetate. The combined organic layers were extracted 3 times with 100 ml 1 M citric acid, aqueous sodium carbonate and 2 times with 100 ml brine, dried over MgSO₄ and filtrated. The solvent was removed and the residue was purified by distillation resulting in a yield of 85%

### Synthesis of 4-(4-Ethoxycarbonyl-4,5-dihydro-thiazol-2-yl)-piperidine-1-carboxylic acid t-butyl ester

4-formyl-piperidine-1-carboxylic acid tert-butyl ester (1.0 eq, 13 mmol) was dissolved under inert conditions in 40 ml toluene. To this solution L-cystein ethylester hydrochloride (1.6 eq, 21 mmol) and triethylamine (1.6 eq, 21 mmol) were added. The mixture was refluxed for 14 h. The generated water was removed with a Dean & Stark trap.

The solvent was removed and the residue was dissolved in 100 ml ethylacetate. The organic layer was extracted 3 times with 50 ml 1 M citric acid, aqueous potassium hydrogen carbonate and 2 times with 50 ml brine, dried over MgSO₄ and filtrated. The solvent was removed and the residue was purified by silica gel chromatography using a PE/EA 4:1 gradient. Yield: 75%

### Synthesis of 4-(4-Ethoxycarbonyl-thiazol-2-yl)-piperidine-1-carboxylic acid t-butyl ester

4-(4-Ethoxycarbonyl-4,5-dihydro-thiazol-2-yl)-piperidine-1-carboxylic acid tert-butyl ester (1.0 eq, 8.7 mmol) was solved in 160 ml toluene under inert conditions. To this solution MnO₂ (15.0 eq, 130 mmol) was added. The reaction was heated to 70° C. under stirring for 5 h. The mixture was filtered over celite and the filtration agent was washed 3 times with 30 ml toluene and ethylacetate. The combined organic layers were distilled in vacuo. The residue was purified by silica gel chromatography using a DCM/MeOH 95:5 gradient. Yield: 30%

C-Terminal Functionalisation

4-(4-Ethoxycarbonyl-thiazol-2-yl)-piperidine-1-carboxylic acid tert-butyl ester (1.0 eq, 2.9 mmol) was dissolved under inert gas in 40 ml dioxane. Under stirring 1.5 ml aqueous 2 N NaOH was added dropwise over a period of 10 min. Afterwards the mixture was stirred for 2 h at r.t.

The reaction was neutralized with 2 N HCl and the solvent was evaporated in vacuo. The residue was dissolved in 50 ml ethylacetate. The organic layer was extracted 3 times with 10 ml of 1 M citric acid and water, dried over MgSO₄ and filtered. The solvent was removed and the residue was dried in vacuo. Yield 95%

4-(4-Carboxy-thiazol-2-yl)-piperidine-1-carboxylic acid tert-butyl ester (1 eq) was dissolved under inert conditions in dry dimethylacetamide (0.03 mmol/ml). To this solution aryl- or alkylamine (1 eq), diisopropylethylamine (2 eq) and O-benzotriazol-1-yl-N,N,N′,N′-tetramethyluronium hexafluorophosphate (2 eq) was added. The reaction mixture was stirred for 12 h at r.t.

The solvent was removed in vacuo and the residue was dissolved in ethylacetate. The organic layer was extracted 3 times with 1 M citric acid, aqueous potassium hydrogen carbonate and 2 times with brine, dried over MgSO₄ and filtred. The solvent was removed and the residue was purified by silica gel chromatography using a DCM/MeOH 95:5 gradient. Yield: 40-80%

N-Terminal Functionalisation

The N-protected substrate was treated under inert condition with 4 M HCl/dioxane (conc. 0.03 mmol substrate in 1 mL HCl/dioxane) and was stirred for 2 h at r.t.

The solvent was removed in vacuo to yield the HCl salt of the free amine without further purification.

The free amino compound (1 eq) was dissolved under inert conditions in dry dimethylacetamide (0.03 mmol/ml). To this solution aryl- or alkylcarboxylic acid (1 eq), diisopropylethylamine (2 eq) and O-benzotriazol-1-yl-N,N,N′,N′-tetramethyluronium hexafluorophosphate (2 eq) was added in this sequence and the reaction mixture was stirred for 12 h at r.t.

The solvent was removed in vacuo and the residue was dissolved in ethylacetate. The organic layer was extracted 3 times with 1 M citric acid, aqueous potassium hydrogen carbonate and 2 times with brine, dried over MgSO₄ and filtred. The solvent was removed and the residue was purified by silica gel chromatography using a DCM/MeOH 95:5 gradient. Yield: 40-80%

General Synthesis for Compounds of Type (III) and (II)

Procedure for the Synthesis of Compounds of type (IIIa), (IIIc), (IIa) and (IIb)

7.3×10⁻⁴ mol of the benzoic acid derivative (VI) was dissolved in 5 ml DMF and 1 eq. of Hünig's base was added, stirring the reaction mixture for a few minutes, followed by the addition of 1 eq. of HBTU and further stirring at r.t. for 2 min. Afterwards 1 eq. 2-Amino-thiazole-4-carboxylic acid ethyl ester was added, stirring the mixture overnight at the same temperature. Subsequently, the solvent was removed by filtration and the residue redissolved in 5 ml dioxane and treated with 0.5 ml of a 1M NaOH solution. After the reaction was complete, the pH was decreased to 1-2 with a 1M HCl solution and the precipitated product (VII) filtered and dried in vacuo. For the next step, intermediate (VII) was dissolved in 3 ml DMF and 1 eq. of Hünig's base was added, stirring the reaction mixture for a few minutes, followed by the addition of 1 eq. of HBTU and further stirring at r.t. for 2 min. Afterwards 1 eq. the amino component was added, stirring the mixture overnight at the same temperature. Subsequently, the solvent was removed by filtration and the crude product redissolved in 10 ml ethyl acetate, washed twice with 10 ml citric acid (1M solution), 10 ml sat. NaHCO₃ solution and 10 ml water. The organic phase was then evaporated and the residue dried over MgSO₄. The solvent was removed and final purification was realised by preparative HPLC as described above.

As a second variante for the first step, the corresponding acid chloride derivative of (VI) could be reacted with the 2-Amino-thiazole-4-carboxylic acid ethyl ester (1:1) using 1,1 eq. of Hünig's base.

Procedure for the Synthesis of Compounds of type (IIIb, d, e, f) and (IIc, d)

6.3×10⁻⁴ mol of 2-Bromo-thiazole-4-carboxylic acid ethyl ester (X) was dissolved in 10 ml THF together with 2.2 eq. of the respective piperazine (IX), allowing to reflux overnight. Afterwards the solvent was removed in vacuo and the residue purified by pTLC (PE/EE 2/1).

The second and the third step of the reaction were accomplished as described above under the procedure for the synthesis of compounds of type (IIIa) and (IIIc).

For a synthesis-protocol of type (IIIb, d, e, f and IIc, d) compounds with Z═CH, see WO 2004/058750.

Exemplary compounds of formula (I) of the present invention include, but are not limited to, the following:

| | | | LC/(+)- | |
|---|---|---|---|---|
| Cp. | Name | Mass | ESI-MS: | ¹H-NMR |
| 1 | [4-(1H-Indol-3-yl)-piperidin- | 501 | 502 [M + H]⁺ | δ = 1.69-2.04 (m, 4 H, 2 CH₂), |
| | 1-yl]-{2-[1-(pyrazine-2- | | | 2.07-2.24 (m, 4 H, 2 CH₂), |
| | carbonyl)-piperidin-4-yl]- | | | 2.96-3.4 (m, 6 H, 2 CH₂, 2 CH), |
| | thiazol-4-yl}-methanone | | | 4.05-4.91 (m, 4 H, 2 CH₂), |
| | | | | 6.99-7.78 (m, 6 H, CH_{Ar}), |
| | | | | 8.01 (s, 1 H, NH), 8.5-8.9 (m, 3 H, |
| | | | | CH_{Ar}). |
| 2 | 4-[5-(4-{4-[4-(1H-Indol-3-yl)- | 621 | 622 [M + H]⁺ | |
| | piperidine-1-carbonyl]- | | | |
| | thiazol-2-yl}-piperidin-1-yl)- | | | |
| | 5-oxo-pentyl]-tetrahydro- | | | |
| | thieno[3,4-d]imidazol-2-one | | | |
| 3 | 4-[4-(4-Benzhydryl- | 547 | 548 [M + H]⁺ | |
| | piperazine-1-carbonyl)- | | | |
| | thiazol-2-yl]-piperidine-1- | | | |
| | carboxylic acid tert-butyl ester | | | |
| 4 | 2-Phenyl-1-(4-{4-[4-(3- | 543 | 544 [M + H]⁺ | δ = 1.48-1.69 (m, 2 H, CH₂), |
| | trifluoromethyl-phenyl)- | | | 2.02-2.13 (m, 2 H, CH₂), |
| | piperazine-1-carbonyl]- | | | 2.70-2.9 (m, 2 H, CH₂), |
| | thiazol-2-yl}-piperidin-1-yl)- | | | 3.22-3.38 (m, 5 H, 2 CH₂), 1 CH), 3.74 (s, |
| | ethanone | | | 2 H, CH₂), 4.51-4.60 (m, 2 H, 1 |
| | | | | CH₂), 7.06-7.91 (m, 10 H |
| | | | | CH_{Ar}). |
| 5 | 3-[2-(4-{4-[4-(1H-Indol-3-yl)- | 597 | 598 [M + H]⁺ | |
| | piperidine-1-carbonyl]- | | | |
| | thiazol-2-yl}-piperidin-1-yl)- | | | |
| | 2-oxo-ethyl]-5-methoxy- | | | |
| | indan-1-one | | | |
| 6 | 2-(4-Fluoro-phenyl)-1-(4-{4- | 561 | 562 [M + H]⁺ | δ = 1.58-1.7 (m, 2 H, CH₂), |
| | [4-(3-trifluoromethyl-phenyl)- | | | 2.06-2.18 (m, 2 H, CH₂), |
| | piperazine-1-carbonyl]- | | | 2.75 (s, 2 H, CH₂) 2.78-2.9 (m, 2 H, |
| | thiazol-2-yl}-piperidin-1-yl)- | | | CH₂), 3.27-3.38 (m, 5 H, 2 |
| | ethanone | | | CH₂, 1 CH), 4.5-4.61 (m, 2 H, |
| | | | | CH₂), 7.02-7.94 (m, 9 H CH_{Ar}). |
| 7 | 2,2-Diphenyl-1-(4-{4-[4-(3- | 619 | 620 [M + H]⁺ | δ = 1.3-1.75 (m, 2 H, CH₂) |
| | trifluoromethyl-phenyl)- | | | 1.94-2.19 (m, 2 H, CH₂), |
| | piperazine-1-carbonyl]- | | | 2.9-3.02 (m, 2 H, CH₂) |
| | thiazol-2-yl}-piperidin-1-yl)- | | | 3.18-3.43 (m, 6 H, 2 CH, 2 CH₂) |
| | ethanone | | | 4.18-4.7 (m, 2 H, CH₂), 7.11-7.95 (m, |
| | | | | 15 H, CH_{Ar}). |
| 8 | 1-(4-{4-[4-(1H-Indol-3-yl)- | 603 | 604 [M + H]⁺ | |
| | piperidine-1-carbonyl]- | | | |
| | thiazol-2-yl}-piperidin-1-yl)- | | | |
| | 3,3-diphenyl-propan-1-one | | | |
| 9 | 4-{4-[4-(3-Trifluoromethyl- | 525 | 526 [M + H]⁺ | δ = 1.45 (s, 9 H, 3 CH₃), |
| | phenyl)-piperazine-1- | | | 1.65-1.78 (m, 2 H, CH₂), |
| | carbonyl]-thiazol-2-yl}- | | | 2.05-2.18 (m, 2 H, CH₂), 2.91-3.03 (m, 2 |
| | piperidine-1-carboxylic acid | | | H, CH₂), 3.29 (m_{c,} 1 H, CH), |
| | tert-butyl ester | | | 3.38 (m_{c}, 1 H, 2 CH₂) |
| | | | | 4.10-4.19 (m, 2 H, CH₂), |
| | | | | 7.10-7.96 (m, 5 H, CH_{Ar}) |
| 10 | (2-Piperidin-4-yl-thiazol-4- | 424 | 425 [M + H]⁺ | |
| | yl)-[4-(3-trifluoromethyl- | | | |
| | phenyl)-piperazin-1-yl]- | | | |
| | methanone | | | |
| 11 | {2-[1-(4,7-Dimethyl- | 569 | 570 [M + H]⁺ | δ = 1.75-2.07 (m, 4 H, 2 CH₂), |
| | pyrazolo[5,1-c][1,2,4]triazine- | | | 2.08-2.42 (m, 4 H, 2 CH₂), |
| | 3-carbonyl)-piperidin-4-yl]- | | | 2.63 (s, 3 H, CH₃), 2.87 (s, 3 H, |
| | thiazol-4-yl}-[4-(1H-indol-3- | | | CH₃), 2.94-3.4 (m, 6 H, 2 CH₂, |
| | yl)-piperidin-1-yl]-methanone | | | 2 CH), 3.83 (m_{C,} 1 H, CH₂), |
| | | | | 4.44-4.6 (m, 1 H, CH₂) |
| | | | | 4.84 (m_{c,} 2 H, CH₂), 6.98-7.79 (m, 7 |
| | | | | H, CH_{Ar}), 8.02 (s, 1 H, NH). |
| 12 | 1-(4-{4-[4-(1H-Indol-3-yl)- | 513 | 514 [M + H]⁺ | δ = 1.65-1.93 (m, 4 H, 2 CH₂), |
| | piperidine-1-carbonyl]- | | | 1.96-2.24 (m, 4 H, 2 CH₂), |
| | thiazol-2-yl}-piperidin-1-yl)- | | | 2.77-2.91 (m, 2 H, CH₂), |
| | 2-phenyl-ethanone | | | 2.93-3.05 (m, 1 H, CH₂), |
| | | | | 3.07-3.2 (m, 2 H, 2 CH) 3.25-3.34 (m, 1 |
| | | | | H, CH₂), 3.76 (s, 2 H, CH₂), |
| | | | | 3.94 (m_{c,} 1 H, CH₂), |
| | | | | 4.43-4.57 (m, 1 H, CH₂), 4.66 (m_{c,} 1 H, |
| | | | | CH₂), 4.75-4.90 (m, 1 H, CH₂), |
| | | | | 6.98-7.75 (m, 11 H, CH_{Ar}), |
| | | | | 7.98 (s, 1 H, NH). |
| 13 | 5-Methoxy-3-[2-oxo-2-(4-{4- | 627 | 628 [M + H]⁺ | |
| | [4-(3-trifluoromethyl-phenyl)- | | | |
| | piperazine-1-carbonyl]- | | | |
| | thiazol-2-yl}-piperidin-1-yl)- | | | |
| | ethyl]-indan-1-one | | | |
| 14 | 1-(4-Fluoro-phenyl)-2-(4-{4- | 531 | 532 [M + H]⁺ | δ = 1.58-1.85 (m, 4 H, 2 CH₂), |
| | [4-(1H-indol-3-yl)-piperidine- | | | 2.07-2.19 (m, 4 H, 2 CH₂), |
| | 1-carbonyl]-thiazol-2-yl}- | | | 2.64-3.64 (m, 6 H, 2 CH₂, 2 |
| | piperidin-1-yl)-ethanone | | | CH), 3.97 (m_{C,} 1 H, CH₂), |
| | | | | 4.41 (m_{C,} 2 H, CH₂) 4.49-4.7 (m, 1 |
| | | | | H, CH₂) 5.48 (s, 2 H, CH₂), |
| | | | | 6.84-7.71 (m, 10 H CH_{Ar}). |
| 15 | 3-Phenyl-1-(4-{4-[4-(3- | 553 | 554 [M + H]⁺ | δ = 1.71-1.97 (m 2 H, CH₂), |
| | trifluoromethyl-phenyl)- | | | 2.20-2.35 (m, 2 H, CH₂), |
| | piperazine-1-carbonyl]- | | | 3.02 (m_{c}, 1 H, CH) 3.42-3.55 (m, 6 |
| | thiazol-2-yl}-piperidin-1-yl)- | | | H, 2 CH₂), 4.51-4.61 (m, 2 H, |
| | propynone | | | CH₂), 7.2-8.0 (m, 9 H, CH_{Ar}). |
| 16 | 3,3-Diphenyl-1-(4-{4-[4-(3- | 633 | 634 [M + H]⁺ | δ = 1.44-1.59 (m, 2 H, CH₂) |
| | trifluoromethyl-phenyl)- | | | 2.04-2.12 (m, 2 H, CH₂), |
| | piperazine-1-carbonyl]- | | | 2.7-2.83 (m, 2 H, CH₂), |
| | thiazol-2-yl}-piperidin-1-yl)- | | | 3.08-3.34 (m, 4 H, 2 CH, CH₂), |
| | propan-1-one | | | 3.34-3.41 (m, 4 H, 2 CH₂), 4.46-463 (m, |
| | | | | 2 H, CH₂), 7.11-7.95 (m, 15 H, |
| | | | | CH_{Ar}). |
| 17 | {2-[1-(Pyrazine-2-carbonyl)- | 531 | 532 [M + H]⁺ | δ = 1.83-1.99 (m, 2 H, CH₂), |
| | piperidin-4-yl]-thiazol-4-yl}- | | | 2.07-2.35 (m, 2 H, CH₂), |
| | [4-(3-trifluoromethyl-phenyl)- | | | 2.73-2.95 (m, 2 H, CH₂), 3.13 (m_{c}, 1 |
| | piperazin-1-yl]-methanone | | | H, CH), 3.33-3.44 (m, 4 H, 2 |
| | | | | CH₂), 4.6-4.74 (m, 2 H, CH₂), |
| | | | | 7.11-8.88 (m, 8 H, CH_{Ar}). |
| 18 | {2-[1-(4,7-Dimethyl- | 599 | 600 [M + H]⁺ | |
| | pyrazolo[5,1-c][1,2,4]triazine- | | | |
| | 3-carbonyl)-piperidin-4-yl]- | | | |
| | thiazol-4-yl}-[4-(3- | | | |
| | trifluoromethyl-phenyl)- | | | |
| | piperazin-1-yl]-methanone | | | |
| 19 | 4-(4-{2-[4-(1H-Indol-3-yl)- | 495 | 496 [M + H]⁺ | δ = 1.46 (s 9 H 3 CH₃), |
| | piperidin-1-carbonyl]}- | | | 1.67-1.92 (m, 4 H, 2 CH₂), |
| | thiazol-2-yl)-piperidine-1- | | | 2.06-2.23 (m, 4 H, 2 CH₂), |
| | carboxylic acid tert-butyl ester | | | 2.84-2.95 (m, 2 H, 1 CH₂), |
| | | | | 2.97-3.08 (m, 1 H, 1 CH₂), |
| | | | | 3.1-3.22 (m, 2 H, 2 CH) 3.23-3.36 (m, 1 |
| | | | | H, 1 CH₂), 4.11-4.23 (m, 2 H, 1 |
| | | | | CH₂), 4.5-4.91 (m, 2 H 1 CH₂), |
| | | | | 6.96-7.77 (m, 6 H CH_{Ar}), |
| | | | | 7.98 (s 1 H, NH). |
| 20 | 1-{4-[4-(4-Benzhydryl- | 583 | 584 [M + H]⁺ | |
| | piperazine-1-carbonyl)- | | | |
| | thiazol-2-yl]-piperidin-1-yl}- | | | |
| | 2-(4-fluoro-phenyl)-ethanone | | | |
| 21 | (4-Benzhydryl-piperazin-1- | 621 | 622 [M + H]⁺ | |
| | yl)-{2-[1-(4,7-dimethyl- | | | |
| | pyrazolo[5,1-c][1,2,4]triazine- | | | |
| | 3-carbonyl)-piperidin-4-yl]- | | | |
| | thiazol-4-yl}-methanone | | | |
| 22 | (4-Benzhydryl-piperazin-1- | 447 | 448 [M + H]⁺ | |
| | yl)-(2-piperidin-4-yl-thiazol-4- | | | |
| | yl)-methanone | | | |
| 23 | 1-{4-[4-(4-Benzhydryl- | 565 | 566 [M + H]⁺ | δ = 1.35 (m_{C,} 1 H, CH₂), |
| | piperazine-1-carbonyl)- | | | 1.60 (m_{C,} 1 H, CH₂), 1.93 (m_{C,} 2 H, |
| | thiazol-2-yl]-piperidin-1-yl}- | | | CH₂), 2.37 (m_{C,} 4 H, 2 CH₂), |
| | 2-phenyl-ethanone | | | 2.71 (m_{C,} 1 H, CH₂), |
| | | | | 2.97-3.12 (m, 2 H, CH₂, CH), 3.67 (s, 2 |
| | | | | H, CH₂), 3.69-3.91 (m, 5 H, 6 |
| | | | | CH₂), 4.18 (s, 1 H, CH), |
| | | | | 4.58 (m_{C}, 1 H, CH₂), 7.09-7.68 (m, |
| | | | | 16 H, CH_{Ar}). |
| 24 | 3-(2-{4-[4-(4-Benzhydryl- | 649 | 650 [M + H]⁺ | |
| | piperazine-1-carbonyl)- | | | |
| | thiazol-2-yl]-piperidin-1-yl}- | | | |
| | 2-oxo-ethyl)-5-methoxy- | | | |
| | indan-1-one | | | |

Exemplary compounds of formula (I) of the present invention include, but are not limited to, the following:

| | | | LC/(+)-ESI- | Biological |
|---|---|---|---|---|
| Cp. | Name | Mass | MS: | activity¹⁾ |
| 29 | {2-[1-(2,5-Dimethoxy-phenyl)-piperidin-4- | 560 | 561 [M + H]⁺ | ++ |
| | yl]-thiazol-4-yl}-[4-(3-trifluoromethyl- | | | |
| | phenyl)-piperazin-1-yl]-methanone | | | |
| 35 | {2-[1-(3,3-Diphenyl-propyl)-piperidin-4- | 618 | 619 [M + H]⁺ | +++ |
| | yl]-thiazol-4-yl}-[4-(3-trifluoromethyl- | | | |
| | phenyl)-piperazin-1-yl]-methanone | | | |
| 48 | {4-[1-(4-Fluoro-benzyl)-1H- | 692 | 693 [M + H]⁺ | +++ |
| | benzoimidazol-2-yl]-piperazin-1-yl}-{2-[1- | | | |
| | (2-trifluoromethoxy-benzoyl)-piperidin-4- | | | |
| | yl]-thiazol-4-yl}-methanone | | | |
| 70 | 4-(4-{4-[4-(3-Trifluoromethyl-phenyl)- | 553 | 554 [M + H]⁺ | ++ |
| | piperazine-1-carbonyl]-thiazol-2-yl}- | | | |
| | piperidine-1-carbonyl)-benzonitrile | | | |
| 96 | [4-(1H-Benzoimidazol-2-yl)-piperazin-1- | 618 | 619 [M + H]⁺ | +++ |
| | yl]-{2-[1-(2-trifluoromethoxy-benzoyl)- | | | |
| | piperidin-4-yl]-thiazol-4-yl}-methanone | | | |
| 97 | [4-(5-Methoxy-1H-benzoimidazol-2-yl)- | 583 | 584 [M + H]⁺ | + |
| | piperazin-1-yl]-{2-[1-(2-trifluoromethoxy- | | | |
| | benzoyl)-piperidin-4-yl]-thiazol-4-yl}- | | | |
| | methanone | | | |
| 98 | {4-[1-(4-Fluoro-benzyl)-1H- | 692 | 693 [M + H]⁺ | +++ |
| | benzoimidazol-2-yl]-piperazin-1-yl}-{2-[1- | | | |
| | (2-trifluoromethoxy-benzoyl)-piperidin-4- | | | |
| | yl]-thiazol-4-yl}-methanone | | | |
| 107 | [2-(1-Pyridin-4-ylmethyl-piperidin-4-yl)- | 515 | 516 [M + H]⁺ | + |
| | thiazol-4-yl]-[4-(3-trifluoromethyl-phenyl)- | | | |
| | piperazin-1-yl]-methanone | | | |
| 108 | [2-(1-Pyridin-3-ylmethyl-piperidin-4-yl)- | 446 | 447 [M + H]⁺ | + |
| | thiazol-4-yl]-[4-(3-trifluoromethyl-phenyl)- | | | |
| | piperazin-1-yl]-methanone | | | |
| 109 | {2-[1-(2-Trifluoromethoxy-benzyl)- | 598 | 599 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 110 | 2-[1-(2-Trifluoromethoxy-benzoyl)- | 647 | 648 [M + H]⁺ | + |
| | piperidin-4-yl]-thiazole-4-carboxylic acid | | | |
| | bis-(4-chloro-benzyl)-amide | | | |
| 111 | (4-Benzotriazol-1-yl-piperidin-1-yl)-{2-[1- | 584 | 585 [M + H]⁺ | + |
| | (2-trifluoromethoxy-benzoyl)-piperidin-4- | | | |
| | yl]-thiazol-4-yl}-methanone | | | |
| 112 | 4-(4-{4-[4-(3-Trifluoromethyl-phenyl)- | 525 | 526 [M + H]⁺ | + |
| | piperazine-1-carbonyl]-thiazol-2-yl}- | | | |
| | piperidin-1-yl)-benzonitrile | | | |
| 113 | 4-{4-[4-(3-Trifluoromethyl-phenyl)- | 468 | 469 [M + H]⁺ | + |
| | piperazine-1-carbonyl]-thiazol-2-yl}- | | | |
| | piperidine-1-carboxylic acid | | | |
| 115 | {2-[1-(3-Trifluoromethoxy-benzyl)- | 598 | 599 [M + H]⁺ | ++ |
| | piperidin-4-yl]-thiazol-4-yl}-[4-(3- | | | |
| | trifluoromethyl-phenyl)-piperazin-1-yl]- | | | |
| | methanone | | | |
| 117 | {2-[1-(4-Methanesulfonyl-phenyl)- | 578 | 579 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 118 | [4-(3-Trifluoromethyl-phenyl)-piperazin-1- | 569 | 570 [M + H]⁺ | + |
| | yl]-[2-(4′-trifluoromethyl-3,4,5,6- | | | |
| | tetrahydro-2H-[1,2′]bipyridinyl-4-yl)- | | | |
| | thiazol-4-yl]-methanone | | | |
| 119 | [2-(1-Phenyl-piperidin-4-yl)-thiazol-4-yl]- | 500 | 501 [M + H]⁺ | + |
| | [4-(3-trifluoromethyl-phenyl)-piperazin-1- | | | |
| | yl]-methanone | | | |
| 120 | [2-(1-Benzyl-piperidin-4-yl)-thiazol-4-yl]- | 509 | 510 [M + H]⁺ | + |
| | [4-(4,6-dimethoxy-[1,3,5]triazin-2-yl)- | | | |
| | piperazin-1-yl]-methanone | | | |
| 121 | {2-[1-(4-Bromo-benzyl)-piperidin-4-yl]- | 587 | 588 [M + H]⁺ | + |
| | thiazol-4-yl}-[4-(4,6-dimethoxy- | | | |
| | [1,3,5]triazin-2-yl)-piperazin-1-yl]- | | | |
| | methanone | | | |
| 122 | [2-(1-Pyrazin-2-yl-piperidin-4-yl)-thiazol- | 502 | 503 [M + H]⁺ | + |
| | 4-yl]-[4-(3-trifluoromethyl-phenyl)- | | | |
| | piperazin-1-yl]-methanone | | | |
| 123 | [2-(3,4,5,6-Tetrahydro-2H- | 501 | 502 [M + H]⁺ | ++ |
| | [1,2′]bipyridinyl-4-yl)-thiazol-4-yl]-[4-(3- | | | |
| | trifluoromethyl-phenyl)-piperazin-1-yl]- | | | |
| | methanone | | | |
| 124 | (4-{4-[4-(2-Methylsulfanyl-pyrimidin-4- | 592 | 593 [M + H]⁺ | + |
| | yl)-piperazine-1-carbonyl]-thiazol-2-yl}- | | | |
| | piperidin-1-yl)-(2-trifluoromethoxy- | | | |
| | phenyl)-methanone | | | |
| 126 | 1-(4-{4-[4-(4,6-Dimethoxy-[1,3,5]triazin-2- | 555 | 556 [M + H]⁺ | + |
| | yl)-piperazine-1-carbonyl]-thiazol-2-yl}- | | | |
| | piperidin-1-yl)-2-(4-fluoro-phenyl)- | | | |
| | ethanone | | | |
| 127 | [4-(4,6-Dimethoxy-[1,3,5]triazin-2-yl)- | 607 | 608 [M + H]⁺ | + |
| | piperazin-1-yl]-{2-[1-(2-trifluoromethoxy- | | | |
| | benzoyl)-piperidin-4-yl]-thiazol-4-yl}- | | | |
| | methanone | | | |
| 129 | (4-{4-[4-(7H-Purin-6-yl)-piperazine-1- | 586 | 587 [M + H]⁺ | + |
| | carbonyl]-thiazol-2-yl}-piperidin-1-yl)-(2- | | | |
| | trifluoromethoxy-phenyl)-methanone | | | |
| 130 | (4-{4-[4-(2,6-Dimethoxy-pyrimidin-4-yl)- | 606 | 607 [M + H]⁺ | + |
| | piperazine-1-carbonyl]-thiazol-2-yl}- | | | |
| | piperidin-1-yl)-(2-trifluoromethoxy- | | | |
| | phenyl)-methanone | | | |
| 145 | 2-[1-(2-Trifluoromethoxy-benzoyl)- | 556 | 557 [M + H]⁺ | + |
| | piperidin-4-yl]-thiazole-4-carboxylic acid | | | |
| | [1-(1H-indol-2-yl)-ethyl]-methyl-amide | | | |
| 147 | 2-[1-(2-Trifluoromethoxy-benzoyl)- | 602 | 603 [M + H]⁺ | + |
| | piperidin-4-yl]-thiazole-4-carboxylic acid | | | |
| | methyl-(2-morpholin-4-yl-1-phenyl-ethyl)- | | | |
| | amide | | | |
| 148 | 2-[1-(2-Trifluoromethoxy-benzoyl)- | 533 | 534 [M + H]⁺ | ++ |
| | piperidin-4-yl]-thiazole-4-carboxylic acid | | | |
| | benzyl-(2-hydroxy-ethyl)-amide | | | |
| 149 | 2-[1-(2-Trifluoromethoxy-benzoyl)- | 593 | 594 [M + H]⁺ | + |
| | piperidin-4-yl]-thiazole-4-carboxylic acid | | | |
| | benzyl-phenethyl-amide | | | |
| 151 | (4-{4-[4-(3-Chloro-phenyl)-piperazine-1- | 578 | 579 [M + H]⁺ | + |
| | carbonyl]-thiazol-2-yl}-piperidin-1-yl)-(2- | | | |
| | trifluoromethoxy-phenyl)-methanone | | | |
| 152 | [4-(4,6-Dimethoxy-[1,3,5]triazin-2-yl)- | 419 | 420 [M + H]⁺ | ++ |
| | piperazin-1-yl]-(2-piperidin-4-yl-thiazol-4- | | | |
| | yl)-methanone | | | |
| 153 | {2-[1-(2,4-Dimethoxy-phenyl)-piperidin-4- | 560 | 561 [M + H]⁺ | + |
| | yl]-thiazol-4-yl}-[4-(3-trifluoromethyl- | | | |
| | phenyl)-piperazin-1-yl]-methanone | | | |
| 154 | {2-[1-(4-Trifluoromethoxy-phenyl)- | 584 | 585 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 155 | [4-(3-Trifluoromethyl-phenyl)-piperazin-1- | 568 | 569 [M + H]⁺ | + |
| | yl]-{2-[1-(4-trifluoromethyl-phenyl)- | | | |
| | piperidin-4-yl]-thiazol-4-yl}-methanone | | | |
| 156 | [2-(1-Benzo[1,3]dioxol-5-yl-piperidin-4- | 544 | 545 [M + H]⁺ | + |
| | yl)-thiazol-4-yl]-[4-(3-trifluoromethyl- | | | |
| | phenyl)-piperazin-1-yl]-methanone | | | |
| 157 | {2-[1-(2,3-Dihydro-benzo[1,4]dioxin-6-yl)- | 558 | 559 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 158 | {2-[1-(2-Methoxy-phenyl)-piperidin-4-yl]- | 530 | 531 [M + H]⁺ | + |
| | thiazol-4-yl}-[4-(3-trifluoromethyl-phenyl)- | | | |
| | piperazin-1-yl]-methanone | | | |
| 159 | {2-[1-(3,4-Dimethoxy-phenyl)-piperidin-4- | 560 | 561 [M + H]⁺ | + |
| | yl]-thiazol-4-yl}-[4-(3-trifluoromethyl- | | | |
| | phenyl)-piperazin-1-yl]-methanone | | | |
| 164 | {2-[1-(2,6-Dimethoxy-pyrimidin-4-yl)- | 562 | 563 [M + H]⁺ | ++ |
| | piperidin-4-yl]-thiazol-4-yl}-[4-(3- | | | |
| | trifluoromethyl-phenyl)-piperazin-1-yl]- | | | |
| | methanone | | | |
| 165 | {2-[1-(2-Bromo-benzyl)-piperidin-4-yl]- | 592 | 593 [M + H]⁺ | + |
| | thiazol-4-yl}-[4-(3-trifluoromethyl-phenyl)- | | | |
| | piperazin-1-yl]-methanone | | | |
| 166 | 4-(4-{4-[4-(3-Trifluoromethyl-phenyl)- | 539 | 540 [M + H]⁺ | + |
| | piperazine-1-carbonyl]-thiazol-2-yl}- | | | |
| | piperidin-1-ylmethyl)-benzonitrile | | | |
| 167 | [2-(1-Biphenyl-4-ylmethyl-piperidin-4-yl)- | 590 | 591 [M + H]⁺ | + |
| | thiazol-4-yl]-[4-(3-trifluoromethyl-phenyl)- | | | |
| | piperazin-1-yl]-methanone | | | |
| 168 | {2-[1-(2,6-Difluoro-benzyl)-piperidin-4-yl]- | 550 | 551 [M + H]⁺ | + |
| | thiazol-4-yl}-[4-(3-trifluoromethyl-phenyl)- | | | |
| | piperazin-1-yl]-methanone | | | |
| 169 | {2-[1-(4-Trifluoromethyl-benzyl)-piperidin- | 582 | 583 [M + H]⁺ | + |
| | 4-yl]-thiazol-4-yl}-[4-(3-trifluoromethyl- | | | |
| | phenyl)-piperazin-1-yl]-methanone | | | |
| 170 | {2-[1-(4-Fluoro-benzyl)-piperidin-4-yl]- | 532 | 533 [M + H]⁺ | + |
| | thiazol-4-yl}-[4-(3-trifluoromethyl-phenyl)- | | | |
| | piperazin-1-yl]-methanone | | | |
| 171 | {2-[1-(2-Chloro-4-fluoro-benzyl)-piperidin- | 566 | 567 [M + H]⁺ | + |
| | 4-yl]-thiazol-4-yl}-[4-(3-trifluoromethyl- | | | |
| | phenyl)-piperazin-1-yl]-methanone | | | |
| 172 | {2-[1-(4-Trifluoromethoxy-benzyl)- | 598 | 599 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 173 | {2-[1-(3-Methoxy-benzyl)-piperidin-4-yl]- | 544 | 545 [M + H]⁺ | + |
| | thiazol-4-yl}-[4-(3-trifluoromethyl-phenyl)- | | | |
| | piperazin-1-yl]-methanone | | | |
| 174 | {2-[1-(4-Bromo-benzyl)-piperidin-4-yl]- | 592 | 593 [M + H]⁺ | + |
| | thiazol-4-yl}-[4-(3-trifluoromethyl-phenyl)- | | | |
| | piperazin-1-yl]-methanone | | | |
| 175 | {2-[1-(3,5-Bis-trifluoromethyl-benzyl)- | 650 | 651 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 176 | {2-[1-(4,6-Dimethoxy-[1,3,5]triazin-2-yl)- | 563 | 564 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 177 | [2-(1-Benzyl-piperidin-4-yl)-thiazol-4-yl]- | 514 | 515 [M + H]⁺ | + |
| | [4-(3-trifluoromethyl-phenyl)-piperazin-1- | | | |
| | yl]-methanone | | | |
| 178 | {2-[1-(3-Methoxy-phenyl)-piperidin-4-yl]- | 530 | 531 [M + H]⁺ | + |
| | thiazol-4-yl}-[4-(3-trifluoromethyl-phenyl)- | | | |
| | piperazin-1-yl]-methanone | | | |
| 180 | 2-(4-Fluoro-phenyl)-1-{4-[4-(4-phenyl- | 492 | 493 [M + H]⁺ | + |
| | piperazine-1-carbonyl)-thiazol-2-yl]- | | | |
| | piperidin-1-yl}-ethanone | | | |
| 181 | 2-(4-Fluoro-phenyl)-1-(4-{4-[4-(5- | 600 | 601 [M + H]⁺ | + |
| | trifluoromethyl-benzotriazol-1-yl)- | | | |
| | piperidine-1-carbonyl]-thiazol-2-yl}- | | | |
| | piperidin-1-yl)-ethanone | | | |
| 182 | 1-{4-[4-(4-Benzotriazol-1-yl-piperidine-1- | 532 | 533 [M + H]⁺ | + |
| | carbonyl)-thiazol-2-yl]-piperidin-1-yl}-2- | | | |
| | (4-fluoro-phenyl)-ethanone | | | |
| 183 | 2-(4-{4-[4-(3-Trifluoromethyl-phenyl)- | 573 | 574 [M + H]⁺ | + |
| | piperazine-1-carbonyl]-thiazol-2-yl}- | | | |
| | piperidine-1-carbonyl)-nicotinic acid | | | |
| 184 | 2-(4-{4-[4-(3-Trifluoromethyl-phenyl)- | 616 | 617 [M + H]⁺ | + |
| | piperazine-1-carbonyl]-thiazol-2-yl}- | | | |
| | piperidine-1-carbonyl)-terephthalic acid | | | |
| 185 | 2-(4-{4-[4-(3-Trifluoromethyl-phenyl)- | 572 | 573 [M + H]⁺ | + |
| | piperazine-1-carbonyl]-thiazol-2-yl}- | | | |
| | piperidine-1-carbonyl)-benzoic acid | | | |
| 186 | 3-Hydroxy-2-(4-{4-[4-(3-trifluoromethyl- | 588 | 589 [M + H]⁺ | + |
| | phenyl)-piperazine-1-carbonyl]-thiazol-2- | | | |
| | yl}-piperidine-1-carbonyl)-benzoic acid | | | |
| 187 | 4,5-Dichloro-2-(4-{4-[4-(3-trifluoromethyl- | 640 | 641 [M + H]⁺ | + |
| | phenyl)-piperazine-1-carbonyl]-thiazol-2- | | | |
| | yl}-piperidine-1-carbonyl)-benzoic acid | | | |
| 188 | 2-(4-{4-[4-(3-Trifluoromethyl-phenyl)- | 562 | 563 [M + H]⁺ | + |
| | piperazine-1-carbonyl]-thiazol-2-yl}- | | | |
| | piperidine-1-carbonyl)-cyclopent-1- | | | |
| | enecarboxylic acid | | | |
| 189 | 2-(4-{4-[4-(3-Trifluoromethyl-phenyl)- | 578 | 579 [M + H]⁺ | + |
| | piperazine-1-carbonyl]-thiazol-2-yl}- | | | |
| | piperidine-1-carbonyl)- | | | |
| | cyclohexanecarboxylic acid | | | |
| 190 | 2-(4-{4-[4-(3-Trifluoromethyl-phenyl)- | 576 | 577 [M + H]⁺ | + |
| | piperazine-1-carbonyl]-thiazol-2-yl}- | | | |
| | piperidine-1-carbonyl)-cyclohex-1- | | | |
| | enecarboxylic acid | | | |
| 191 | 4-Oxo-4-(4-{4-[4-(3-trifluoromethyl- | 522 | 523 [M + H]⁺ | + |
| | phenyl)-piperazine-1-carbonyl]-thiazol-2- | | | |
| | yl}-piperidin-1-yl)-but-2-enoic acid | | | |
| 192 | {2-[1-(3,5-Dimethyl-isoxazole-4-sulfonyl)- | 583 | 584 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 193 | [2-(1-Pentafluorobenzenesulfonyl- | 654 | 655 [M + H]⁺ | + |
| | piperidin-4-yl)-thiazol-4-yl]-[4-(3- | | | |
| | trifluoromethyl-phenyl)-piperazin-1-yl]- | | | |
| | methanone | | | |
| 194 | {2-[1-(Thiophene-2-sulfonyl)-piperidin-4- | 570 | 571 [M + H]⁺ | + |
| | yl]-thiazol-4-yl}-[4-(3-trifluoromethyl- | | | |
| | phenyl)-piperazin-1-yl]-methanone | | | |
| 195 | {2-[1-(2-Chloro-4-fluoro-benzenesulfonyl)- | 616 | 617 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 196 | {2-[1-(2-Bromo-benzenesulfonyl)- | 642 | 643 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 197 | {2-[1-(6-Chloro-imidazo[2,1-b]thiazole-5- | 644 | 645 [M + H]⁺ | + |
| | sulfonyl)-piperidin-4-yl]-thiazol-4-yl}-[4- | | | |
| | (3-trifluoromethyl-phenyl)-piperazin-1-yl]- | | | |
| | methanone | | | |
| 198 | [2-(1-Phenylmethanesulfonyl-piperidin-4- | 578 | 579 [M + H]⁺ | + |
| | yl)-thiazol-4-yl]-[4-(3-trifluoromethyl- | | | |
| | phenyl)-piperazin-1-yl]-methanone | | | |
| 199 | {2-[1-(4-Bromo-benzenesulfonyl)- | 642 | 643 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 200 | {2-[1-(2,4-Dichloro-benzenesulfonyl)- | 632 | 633 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 201 | {2-[1-(3,5-Bis-trifluoromethyl- | 700 | 701 [M + H]⁺ | ++ |
| | benzenesulfonyl)-piperidin-4-yl]-thiazol-4- | | | |
| | yl}-[4-(3-trifluoromethyl-phenyl)- | | | |
| | piperazin-1-yl]-methanone | | | |
| 202 | {2-[1-(5-Chloro-2-methoxy- | 628 | 629 [M + H]⁺ | ++ |
| | benzenesulfonyl)-piperidin-4-yl]-thiazol-4- | | | |
| | yl}-[4-(3-trifluoromethyl-phenyl)- | | | |
| | piperazin-1-yl]-methanone | | | |
| 203 | {2-[1-(5-Bromo-thiophene-2-sulfonyl)- | 648 | 649 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 204 | {2-[1-(Toluene-4-sulfonyl)-piperidin-4-yl]- | 578 | 579 [M + H]⁺ | + |
| | thiazol-4-yl}-[4-(3-trifluoromethyl-phenyl)- | | | |
| | piperazin-1-yl]-methanone | | | |
| 205 | {2-[1-(4-Trifluoromethoxy- | 648 | 649 [M + H]⁺ | + |
| | benzenesulfonyl)-piperidin-4-yl]-thiazol-4- | | | |
| | yl}-[4-(3-trifluoromethyl-phenyl)- | | | |
| | piperazin-1-yl]-methanone | | | |
| 206 | {2-[1-(4-Chloro-benzenesulfonyl)- | 598 | 599 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 207 | N-[4-(4-{4-[4-(3-Trifluoromethyl-phenyl)- | 621 | 622 [M + H]⁺ | + |
| | piperazine-1-carbonyl]-thiazol-2-yl}- | | | |
| | piperidine-1-sulfonyl)-phenyl]-acetamide | | | |
| 208 | [2-(1-Benzenesulfonyl-piperidin-4-yl)- | 564 | 565 [M + H]⁺ | + |
| | thiazol-4-yl]-[4-(3-trifluoromethyl-phenyl)- | | | |
| | piperazin-1-yl]-methanone | | | |
| 209 | {2-[1-(3-Methoxy-benzoyl)-piperidin-4-yl]- | 558 | 559 [M + H]⁺ | + |
| | thiazol-4-yl}-[4-(3-trifluoromethyl-phenyl)- | | | |
| | piperazin-1-yl]-methanone | | | |
| 210 | {2-[1-(4-Methyl-3,4-dihydro-2H- | 599 | 600 [M + H]⁺ | ++ |
| | benzo[1,4]oxazine-7-carbonyl)-piperidin-4- | | | |
| | yl]-thiazol-4-yl}-[4-(3-trifluoromethyl- | | | |
| | phenyl)-piperazin-1-yl]-methanone | | | |
| 211 | {2-[1-(Quinoxaline-6-carbonyl)-piperidin- | 580 | 581 [M + H]⁺ | + |
| | 4-yl]-thiazol-4-yl}-[4-(3-trifluoromethyl- | | | |
| | phenyl)-piperazin-1-yl]-methanone | | | |
| 212 | {2-[1-(Thiophene-2-carbonyl)-piperidin-4- | 534 | 535 [M + H]⁺ | + |
| | yl]-thiazol-4-yl}-[4-(3-trifluoromethyl- | | | |
| | phenyl)-piperazin-1-yl]-methanone | | | |
| 213 | {2-[1-(Benzo[b]thiophene-2-carbonyl)- | 584 | 585 [M + H]⁺ | ++ |
| | piperidin-4-yl]-thiazol-4-yl}-[4-(3- | | | |
| | trifluoromethyl-phenyl)-piperazin-1-yl]- | | | |
| | methanone | | | |
| 214 | 3-Phenyl-1-(4-{4-[4-(3-trifluoromethyl- | 554 | 555 [M + H]⁺ | ++ |
| | phenyl)-piperazine-1-carbonyl]-thiazol-2- | | | |
| | yl}-piperidin-1-yl)-propenone | | | |
| 215 | {2-[1-(3-Fluoro-4-trifluoromethyl- | 614 | 615 [M + H]⁺ | +++ |
| | benzoyl)-piperidin-4-yl]-thiazol-4-yl}-[4- | | | |
| | (3-trifluoromethyl-phenyl)-piperazin-1-yl]- | | | |
| | methanone | | | |
| 216 | {2-[1-(5-tert-Butyl-2-methyl-2H-pyrazole- | 588 | 589 [M + H]⁺ | ++ |
| | 3-carbonyl)-piperidin-4-yl]-thiazol-4-yl}- | | | |
| | [4-(3-trifluoromethyl-phenyl)-piperazin-1- | | | |
| | yl]-methanone | | | |
| 217 | {2-[1-(2,3-Dihydro-benzo[1,4]dioxine-2- | 586 | 587 [M + H]⁺ | +++ |
| | carbonyl)-piperidin-4-yl]-thiazol-4-yl}-[4- | | | |
| | (3-trifluoromethyl-phenyl)-piperazin-1-yl]- | | | |
| | methanone | | | |
| 218 | (2-{1-[5-(4-Chloro-phenyl)-2- | 696 | 697 [M + H]⁺ | +++ |
| | trifluoromethyl-furan-3-carbonyl]- | | | |
| | piperidin-4-yl}-thiazol-4-yl)-[4-(3- | | | |
| | trifluoromethyl-phenyl)-piperazin-1-yl]- | | | |
| | methanone | | | |
| 219 | {2-[1-(3,5-Dimethyl-isoxazole-4-carbonyl)- | 547 | 548 [M + H]⁺ | + |
| | piperidin-4-yl]-thiazol-4-yl}-[4-(3- | | | |
| | trifluoromethyl-phenyl)-piperazin-1-yl]- | | | |
| | methanone | | | |
| 220 | (2-{1-[4-(4-Chloro-benzenesulfonyl)-3- | 722 | 723 [M + H]⁺ | + |
| | methyl-thiophene-2-carbonyl]-piperidin-4- | | | |
| | yl}-thiazol-4-yl)-[4-(3-trifluoromethyl- | | | |
| | phenyl)-piperazin-1-yl]-methanone | | | |
| 221 | {2-[1-(4-Methyl-[1,2,3]thiadiazole-5- | 550 | 551 [M + H]⁺ | + |
| | carbonyl)-piperidin-4-yl]-thiazol-4-yl}-[4- | | | |
| | (3-trifluoromethyl-phenyl)-piperazin-1-yl]- | | | |
| | methanone | | | |
| 222 | {2-[1-(Pyridine-3-carbonyl)-piperidin-4- | 529 | 530 [M + H]⁺ | + |
| | yl]-thiazol-4-yl}-[4-(3-trifluoromethyl- | | | |
| | phenyl)-piperazin-1-yl]-methanone | | | |
| 223 | {2-[1-(2-Trifluoromethoxy-benzoyl)- | 612 | 613 [M + H]⁺ | +++ |
| | piperidin-4-yl]-thiazol-4-yl}-[4-(3- | | | |
| | trifluoromethyl-phenyl)-piperazin-1-yl]- | | | |
| | methanone | | | |
| 224 | 3-Cyclopentyl-1-(4-{4-[4-(3- | 548 | 549 [M + H]⁺ | + |
| | trifluoromethyl-phenyl)-piperazine-1- | | | |
| | carbonyl]-thiazol-2-yl}-piperidin-1-yl)- | | | |
| | propan-1-one | | | |
| 225 | [4-(3-Trifluoromethyl-phenyl)-piperazin-1- | 618 | 619 [M + H]⁺ | + |
| | yl]-{2-[1-(3,4,5-trimethoxy-benzoyl)- | | | |
| | piperidin-4-yl]-thiazol-4-yl}-methanone | | | |
| 226 | {2-[1-(3-Dimethylamino-benzoyl)- | 571 | 572 [M + H]⁺ | ++ |
| | piperidin-4-yl]-thiazol-4-yl}-[4-(3- | | | |
| | trifluoromethyl-phenyl)-piperazin-1-yl]- | | | |
| | methanone | | | |
| 229 | 2-(4-Fluoro-phenyl)-1-(4-{4-[4-(2- | 511 | 523 [M + H]⁺ | + |
| | methoxy-phenyl)-piperazine-1-carbonyl]- | | | |
| | thiazol-2-yl}-piperidin-1-yl)-ethanone | | | |
| 230 | {2-[1-(Furan-2-carbonyl)-piperidin-4-yl]- | 518 | 519 [M + H]⁺ | + |
| | thiazol-4-yl}-[4-(3-trifluoromethyl-phenyl)- | | | |
| | piperazin-1-yl]-methanone | | | |
| 231 | 1-{4-[4-(4-Butyl-piperazine-1-carbonyl)- | 472 | 473 [M + H]⁺ | + |
| | thiazol-2-yl]-piperidin-1-yl}-2-(4-fluoro- | | | |
| | phenyl)-ethanone | | | |
| 237 | 2-(4-Fluoro-phenyl)-1-{4-[4-(4-pyridin-2- | 493 | 494 [M + H]⁺ | + |
| | yl-piperazine-1-carbonyl)-thiazol-2-yl]- | | | |
| | piperidin-1-yl}-ethanone | | | |
| 238 | 1-(4-{4-[4-(3-Chloro-phenyl)-piperazine-1- | 526 | 527 [M + H]⁺ | + |
| | carbonyl]-thiazol-2-yl}-piperidin-1-yl)-2- | | | |
| | (4-fluoro-phenyl)-ethanone | | | |

¹⁾The biological data refer to results obtained from the NF-κB inflammation assay.
[“+” stands for 50-80% inhibition, “++” means 80-90% and “+++” stands for 90-100% inhibition]

Exemplary compounds of formula (Ia) of the present invention include, but are not limited to, the following:

| | | | LC/(+)-ESI- | Biological |
|---|---|---|---|---|
| Cp. | Name | Mass | MS: | activity¹⁾ |
| 74 | 4-{4-[4-(3-Trifluoromethyl-phenyl)- | 619 | 620 [M + H]⁺ | ++ |
| | piperazine-1-carbonyl]-thiazol-2-yl}- | | | |
| | piperidine-1-carboxylic acid (7-fluoro-2,3- | | | |
| | dihydro-benzo[1,4]dioxin-5-yl)-amide | | | |
| 75 | 4-{4-[4-(3-Trifluoromethyl-phenyl)- | 601 | 602 [M + H]⁺ | ++ |
| | piperazine-1-carbonyl]-thiazol-2-yl}- | | | |
| | piperidine-1-carboxylic acid (2,3-dihydro- | | | |
| | benzo[1,4]dioxin-6-yl)-amide | | | |
| 76 | 4-{4-[4-(3-Trifluoromethyl-phenyl)- | 615 | 616 [M + H]⁺ | ++ |
| | piperazine-1-carbonyl]-thiazol-2-yl}- | | | |
| | piperidine-1-carboxylic acid (5-methyl-2- | | | |
| | trifluoromethyl-furan-3-yl)-amide | | | |
| 77 | 4-{4-[4-(3-Trifluoromethyl-phenyl)- | 577 | 578 [M + H]⁺ | +++ |
| | piperazine-1-carbonyl]-thiazol-2-yl}- | | | |
| | piperidine-1-carboxylic acid (2-thiophen-2- | | | |
| | yl-ethyl)-amide | | | |
| 78 | 4-{4-[4-(3-Trifluoromethyl-phenyl)- | 509 | 510 [M + H]⁺ | + |
| | piperazine-1-carbonyl]-thiazol-2-yl}- | | | |
| | piperidine-1-carboxylic acid | | | |
| | isopropylamide | | | |
| 79 | 4-{4-[4-(3-Trifluoromethyl-phenyl)- | 627 | 628 [M + H]⁺ | +++ |
| | piperazine-1-carbonyl]-thiazol-2-yl}- | | | |
| | piperidine-1-carboxylic acid (2- | | | |
| | trifluoromethoxy-phenyl)-amide | | | |
| 80 | 4-{4-[4-(3-Trifluoromethyl-phenyl)- | 573 | 574 [M + H]⁺ | + |
| | piperazine-1-carbonyl]-thiazol-2-yl}- | | | |
| | piperidine-1-carboxylic acid (3-methoxy- | | | |
| | phenyl)-amide | | | |
| 81 | 4-{4-[4-(3-Trifluoromethyl-phenyl)- | 633 | 634 [M + H]⁺ | ++ |
| | piperazine-1-carbonyl]-thiazol-2-yl}- | | | |
| | piperidine-1-carboxylic acid (3,4,5- | | | |
| | trimethoxy-phenyl)-amide | | | |
| 114 | 4-(4-{4-[4-(3-Trifluoromethyl-phenyl)- | 568 | 569 [M + H]⁺ | + |
| | piperazine-1-carbonyl]-thiazol-2-yl}- | | | |
| | piperidine-1-carbonyl)-benzonitrile | | | |
| 134 | 4-{4-[4-(3-Trifluoromethyl-phenyl)- | 543 | 544 [M + H]⁺ | + |
| | piperazine-1-carbonyl]-thiazol-2-yl}- | | | |
| | piperidine-1-carboxylic acid phenylamide | | | |
| 142 | 4-{4-[4-(3-Trifluoromethyl-phenyl)- | 561 | 562 [M + H]⁺ | ++ |
| | piperazine-1-carbonyl]-thiazol-2-yl}- | | | |
| | piperidine-1-carboxylic acid (3-fluoro- | | | |
| | phenyl)-amide | | | |
| 143 | 4-{4-[4-(3-Trifluoromethyl-phenyl)- | 561 | 562 [M + H]⁺ | + |
| | piperazine-1-carbonyl]-thiazol-2-yl}- | | | |
| | piperidine-1-carboxylic acid (4-fluoro- | | | |
| | phenyl)-amide | | | |

¹⁾The biological data refer to results obtained from the NF-κB inflammation assay.
[“+” stands for 50-80% inhibition, “++” means 80-90% and “+++” stands for 90-100% inhibition]

Exemplary compounds of formula (Ib) of the present invention include, but are not limited to, the following:

| | | | LC/(+)-ESI- | Biological |
|---|---|---|---|---|
| Cp. | Name | Mass | MS: | activity¹⁾ |
| 103 | 2-{5-Methyl-2-[1-(2-trifluoromethoxy- | 640 | 641 [M + H]⁺ | ++ |
| | benzoyl)-piperidin-4-yl]-thiazol-4-yl}-1-[4- | | | |
| | (3-trifluoromethyl-phenyl)-piperazin-1-yl]- | | | |
| | ethanone | | | |
| 128 | 1-[4-(3-Chloro-phenyl)-piperazin-1-yl]-2- | 606 | 607 [M + H]⁺ | +++ |
| | {5-methyl-2-[1-(2-trifluoromethoxy- | | | |
| | benzoyl)-piperidin-4-yl]-thiazol-4-yl}- | | | |
| | ethanone | | | |

¹⁾The biological data refer to results obtained from the NF-κB inflammation assay.
[“+” stands for 50-80% inhibition, “++” means 80-90% and “+++” stands for 90-100% inhibition]

Exemplary compounds of formula (Ic) of the present invention include, but are not limited to, the following:

| | | | LC/(+)-ESI- | Biological |
|---|---|---|---|---|
| Cp. | Name | Mass | MS: | activity¹⁾ |
| 150 | [1-(5-Chloro-2-methylamino-phenyl)-3,4- | 654 | 655 [M + H]⁺ | ++ |
| | dihydro-1H-isoquinolin-2-yl]-{2-[1-(2- | | | |
| | trifluoromethoxy-benzoyl)-piperidin-4-yl]- | | | |
| | thiazol-4-yl}-methanone | | | |
| 160 | 1-{4-[4-(6,7-Dihydroxy-3,4-dihydro-1H- | 495 | 496 [M + H]⁺ | + |
| | isoquinoline-2-carbonyl)-thiazol-2-yl]- | | | |
| | piperidin-1-yl}-2-(4-fluoro-phenyl)- | | | |
| | ethanone | | | |
| 161 | 1-{4-[4-(6,7-Dimethoxy-3,4-dihydro-1H- | 523 | 524 [M + H]⁺ | + |
| | isoquinoline-2-carbonyl)-thiazol-2-yl]- | | | |
| | piperidin-1-yl}-2-(4-fluoro-phenyl)- | | | |
| | ethanone | | | |
| 163 | 1-{4-[4-(3,4-Dihydro-1H-isoquinoline-2- | 463 | 464 [M + H]⁺ | + |
| | carbonyl)-thiazol-2-yl]-piperidin-1-yl}-2-(4- | | | |
| | fluoro-phenyl)-ethanone | | | |
| 232 | 1-{4-[4-(6,7-Dimethoxy-3-methyl-3,4- | 537 | 538 [M + H]⁺ | + |
| | dihydro-1H-isoquinoline-2-carbonyl)- | | | |
| | thiazol-2-yl]-piperidin-1-yl}-2-(4-fluoro- | | | |
| | phenyl)-ethanone | | | |
| 233 | 1-{4-[4-(6,7-Dihydroxy-1-methyl-3,4- | 509 | 510 [M + H]⁺ | + |
| | dihydro-1H-isoquinoline-2-carbonyl)- | | | |
| | thiazol-2-yl]-piperidin-1-yl}-2-(4-fluoro- | | | |
| | phenyl)-ethanone | | | |
| 234 | 1-{4-[4-(6,7-Dimethoxy-1-methyl-3,4- | 537 | 538 [M + H]⁺ | + |
| | dihydro-1H-isoquinoline-2-carbonyl)- | | | |
| | thiazol-2-yl]-piperidin-1-yl}-2-(4-fluoro- | | | |
| | phenyl)-ethanone | | | |
| 235 | 1-(4-{4-[1-(5-Chloro-2-methylamino- | 602 | 603 [M + H]⁺ | + |
| | phenyl)-3,4-dihydro-1H-isoquinoline-2- | | | |
| | carbonyl]-thiazol-2-yl}-piperidin-1-yl)-2- | | | |
| | (4-fluoro-phenyl)-ethanone | | | |

¹⁾The biological data refer to results obtained from the NF-κB inflammation assay.
[“+” stands for 50-80% inhibition, “++” means 80-90% and “+++” stands for 90-100% inhibition]

Exemplary compounds of formula (II) of the present invention include, but are not limited to, the following:

| | | | LC/(+)-ESI- | Biological |
|---|---|---|---|---|
| Cp. | Name | Mass | MS: | activity¹⁾ |
| 3 | N-{4-[4-(4-Fluoro-phenyl)-piperazine-1- | 494 | 495 [M + H]⁺ | ++ |
| | carbonyl]-thiazol-2-yl}-2-trifluoromethoxy- | | | |
| | benzamide | | | |
| 5 | N-[5-(4-Pyrimidin-2-yl-piperazine-1- | 478 | 479 [M + H]⁺ | + |
| | carbonyl)-thiazol-2-yl]-2-trifluoromethoxy- | | | |
| | benzamide | | | |
| 15 | 2-Methoxy-N-{4-[4-(3-trifluoromethyl- | 490 | 491 [M + H]⁺ | +++ |
| | phenyl)-piperazine-1-carbonyl]-thiazol-2- | | | |
| | yl}-benzamide | | | |
| 16 | 3-Fluoro-4-trifluoromethyl-N-(1-{4-[4-(3- | 643 | 644 [M + H]⁺ | ++ |
| | trifluoromethyl-phenyl)-piperazine-1- | | | |
| | carbonyl]-thiazol-2-yl}piperidin-4- | | | |
| | ylmethyl)-benzamide | | | |
| 17 | 3-Cyclopentyl-N-(1-{4-[4-(3- | 577 | 578 [M + H]⁺ | ++ |
| | trifluoromethyl-phenyl)-piperazine-1- | | | |
| | carbonyl]-thiazol-2-yl}-piperidin- | | | |
| | 4ylmethyl)-propionamide | | | |
| 18 | 2-Trifluoromethoxy-N-(1-{4-[4-(3- | 641 | 642 [M + H]⁺ | ++ |
| | trifluoromethyl-phenyl)-piperazine-1- | | | |
| | carbonyl]-thiazol-2-yl}-piperidin- | | | |
| | 4ylmethyl)-benzamide | | | |
| 19 | 4-Cyano-N-(1-{4-[4-(3-trifluoromethyl- | 582 | 583 [M + H]⁺ | ++ |
| | phenyl)-piperazine-1-carbonyl]-thiazol-2- | | | |
| | yl}-piperidin-4-ylmethyl)-benzamide | | | |
| 20 | [4-(3-Trifluoromethyl-phenyl)-piperazin-1- | 569 | 570 [M + H]⁺ | ++ |
| | yl]-{2-[4-(4-trifluoromethyl-phenyl)- | | | |
| | piperazin-1-yl]-thiazol-4-yl}-methanone | | | |
| 21 | {2-[4-(4-Trifluoromethoxy-phenyl)- | 585 | 586 [M + H]⁺ | + |
| | piperazin-1-yl]-thiazol-4-yl}-[4-(3- | | | |
| | trifluoromethyl-phenyl)-piperazin-1-yl]- | | | |
| | methanone | | | |
| 22 | {2-[4-(2-Trifluoromethoxy-benzyl)- | 599 | 600 [M + H]⁺ | +++ |
| | piperazin-1-yl]-thiazol-4-yl}-[4-(3- | | | |
| | trifluoromethyl-phenyl)-piperazin-1-yl]- | | | |
| | methanone | | | |
| 23 | {2-[4-(4-Bromo-benzyl)-piperazin-1-yl]- | 593 | 594 [M + H]⁺ | +++ |
| | thiazol-4-yl}-[4-(3-trifluoromethyl-phenyl)- | | | |
| | piperazin-1-yl]-methanone | | | |
| 24 | {2-[4-(3-Trifluoromethoxy-benzyl)- | 599 | 600 [M + H]⁺ | +++ |
| | piperazin-1-yl]-thiazol-4-yl}-[4-(3- | | | |
| | trifluoromethyl-phenyl)-piperazin-1-yl]- | | | |
| | methanone | | | |
| 32 | 2-Trifluoromethoxy-N-{4-[4-(3- | 544 | 545 [M + H]⁺ | +++ |
| | trifluoromethyl-phenyl)-piperazine-1- | | | |
| | carbonyl]-thiazol-2-yl}-benzamide | | | |
| 33 | 3-Cyclopentyl-N-{4-[4-(3-trifluoromethyl- | 480 | 481 [M + H]⁺ | +++ |
| | phenyl)-piperazine-1-carbonyl]-thiazol-2- | | | |
| | yl}-propionamide | | | |
| 34 | 3-Fluoro-4-trifluoromethyl-N-{4-[4-(3- | 546 | 547 [M + H]⁺ | +++ |
| | trifluoromethyl-phenyl)-piperazine-1- | | | |
| | carbonyl]-thiazol-2-yl}-benzamide | | | |
| 37 | 3-Cyclopentyl-1-(4-{4-[4-(3- | 549 | 550 [M + H]⁺ | +++ |
| | trifluoromethyl-phenyl)-piperazine-1- | | | |
| | carbonyl]-thiazol-2-yl}-piperazin-1-yl)- | | | |
| | propan-1-one | | | |
| 38 | {2-[4-(2-Methoxy-benzoyl)-piperazin-1- | 559 | 560 [M + H]⁺ | +++ |
| | yl]-thiazol-4-yl}-[4-(3-trifluoromethyl- | | | |
| | phenyl)-piperazin-1-yl]-methanone | | | |
| 39 | {2-[4-(2-Trifluoromethoxy-benzoyl)- | 613 | 614 [M + H]⁺ | +++ |
| | piperazin-1-yl]-thiazol-4-yl}-[4-(3- | | | |
| | trifluoromethyl-phenyl)-piperazin-1-yl]- | | | |
| | methanone | | | |
| 41 | N-{4-[4-(3,4-Dichloro-phenyl)-piperazine- | 544 | 545 [M + H]⁺ | +++ |
| | 1-carbonyl]-thiazol-2-yl}-2- | | | |
| | trifluoromethoxy-benzamide | | | |
| 44 | N-{4-[4-(2-Methoxy-phenyl)-piperazine-1- | 506 | 507 [M + H]⁺ | ++ |
| | carbonyl]-thiazol-2-yl}-2-trifluoromethoxy- | | | |
| | benzamide | | | |
| 55 | 1-(2-Trifluoromethoxy-phenyl)-3-{4-[4-(3- | 559 | 560 [M + H]⁺ | +++ |
| | trifluoromethyl-phenyl)-piperazine-1- | | | |
| | carbonyl]-thiazol-2-yl}-urea | | | |
| 58 | N-[4-(4-Benzhydryl-piperazine-1- | 566 | 567 [M + H]⁺ | ++ |
| | carbonyl)-thiazol-2-yl]-2-trifluoromethoxy- | | | |
| | benzamide | | | |
| 59 | {2-[4-(3,5-Bis-trifluoromethyl-benzoyl)- | 665 | 666 [M + H]⁺ | + |
| | piperazin-1-yl]-thiazol-4-yl}-[4-(3- | | | |
| | trifluoromethyl-phenyl)-piperazin-1-yl]- | | | |
| | methanone | | | |
| 60 | {2-[4-(3-Fluoro-4-trifluoromethyl- | 615 | 616 [M + H]⁺ | +++ |
| | benzoyl)-piperazin-1-yl]-thiazol-4-yl}-[4- | | | |
| | (3-trifluoromethyl-phenyl)-piperazin-1-yl]- | | | |
| | methanone | | | |
| 61 | 4-(4-{4-[4-(3-Trifluoromethyl-phenyl)- | 554 | 555 [M + H]⁺ | +++ |
| | piperazine-1-carbonyl]-thiazol-2-yl}- | | | |
| | piperazine-1-carbonyl)-benzonitrile | | | |
| 62 | 4-(4-{4-[4-(3-Trifluoromethyl-phenyl)- | 540 | 541 [M + H]⁺ | +++ |
| | piperazine-1-carbonyl]-thiazol-2-yl}- | | | |
| | piperazine-1-ylmethyl)-benzonitrile | | | |
| 63 | 4-{4-[4-(3-Trifluoromethyl-phenyl)- | 525 | 526 [M + H]⁺ | ++ |
| | piperazine-1-carbonyl]-thiazol-2-yl}- | | | |
| | piperazine-1-carboxylic acid tert-butyl ester | | | |
| 64 | (2-Piperazin-1-yl-thiazol-4-yl)-[4-(3- | 425 | 426 [M + H]⁺ | + |
| | trifluoromethyl-phenyl)-piperazin-1-yl]- | | | |
| | methanone | | | |
| 65 | {2-[4-(2-Methoxy-phenyl)-piperazin-1-yl]- | 531 | 532 [M + H]⁺ | ++ |
| | thiazol-4-yl}-[4-(3-trifluoromethyl-phenyl)- | | | |
| | piperazin-1-yl]-methanone | | | |
| 66 | 1-[4-(4-{4-[4-(3-Trifluoromethyl-phenyl)- | 543 | 544 [M + H]⁺ | ++ |
| | piperazine-1-carbonyl]-thiazol-2-yl}- | | | |
| | piperazin-1-yl)-phenyl]-ethanone | | | |
| 67 | [4-(3-Trifluoromethyl-phenyl)-piperazin-1- | 569 | 570 [M + H]⁺ | + |
| | yl]-{2-[4-(3-trifluoromethyl-phenyl)- | | | |
| | piperazin-1-yl]-thiazol-4-yl}-methanone | | | |
| 68 | [2-(4-Phenyl-piperazin-1-yl)-thiazol-4-yl]- | 501 | 502 [M + H]⁺ | + |
| | [4-(3-trifluoromethyl-phenyl)-piperazin-1- | | | |
| | yl]-methanone | | | |
| 69 | {2-[4-(4-Fluoro-phenyl)-piperazin-1-yl]- | 519 | 520 [M + H]⁺ | ++ |
| | thiazol-4-yl}-[4-(3-trifluoromethyl-phenyl)- | | | |
| | piperazin-1-yl]-methanone | | | |
| 71 | (4-Benzhydryl-piperazin-1-yl)-[2-(4- | 537 | 538 [M + H]⁺ | +++ |
| | benzyl-piperazin-1-yl)-thiazol-4-yl]- | | | |
| | methanone | | | |
| 72 | [2-(4-Benzyl-piperazin-1-yl)-thiazol-4-yl]- | 515 | 516 [M + H]⁺ | +++ |
| | [4-(3-trifluoromethyl-phenyl)-piperazin-1- | | | |
| | yl]-methanone | | | |
| 99 | 3-Fluoro-4-trifluoromethyl-N-(1-{4-[4-(3- | 642 | 643 [M + H]⁺ | + |
| | trifluoromethyl-phenyl)-piperazine-1- | | | |
| | carbonyl]-thiazol-2-yl}-piperidin-4- | | | |
| | ylmethyl)-benzamide | | | |
| 100 | 3-Cyclopentyl-N-(1-{4-[4-(3- | 576 | 577 [M + H]⁺ | + |
| | trifluoromethyl-phenyl)-piperazine-1- | | | |
| | carbonyl]-thiazol-2-yl}-piperidin-4- | | | |
| | ylmethyl)-propionamide | | | |
| 101 | 2-Trifluoromethoxy-N-(1-{4-[4-(3- | 640 | 641 [M + H]⁺ | + |
| | trifluoromethyl-phenyl)-piperazine-1- | | | |
| | carbonyl]-thiazol-2-yl}-piperidin-4- | | | |
| | ylmethyl)-benzamide | | | |
| 102 | 4-Cyano-N-(1-{4-[4-(3-trifluoromethyl- | 581 | 582 [M + H]⁺ | + |
| | phenyl)-piperazine-1-carbonyl]-thiazol-2- | | | |
| | yl}-piperidin-4-ylmethyl)-benzamide | | | |
| 241 | {2-[1-(2,6-Dimethoxy-pyrimidin-4-yl)- | 576 | 577 [M + H⁺] | ++ |
| | piperidin-4-yl]-5-methoxy-oxazol-4-yl}-[4- | | | |
| | (3-trifluoromethyl-phenyl)-piperazin-1-yl]- | | | |
| | methanone | | | |
| 242 | {2-[1-(4,6-Dimethoxy-[1,3,5]triazin-2-yl)- | 577 | 578 [M + H⁺] | ++ |
| | piperidin-4-yl]-5-methoxy-oxazol-4-yl}-[4- | | | |
| | (3-trifluoromethyl-phenyl)-piperazin-1-yl]- | | | |
| | methanone | | | |
| 243 | {2-[1-(2,6-Dimethoxy-pyrimidin-4-yl)- | 616 | 617 [M + H⁺] | ++ |
| | piperidin-4-yl]-5-methoxy-oxazol-4-yl}-[4- | | | |
| | (5-trifluoromethyl-benzotriazol-1-yl)- | | | |
| | piperidin-1-yl]-methanone | | | |
| 244 | {2-[1-(4,6-Dimethoxy-[1,3,5]triazin-2-yl)- | 617 | 618 [M + H⁺] | ++ |
| | piperidin-4-yl]-5-methoxy-oxazol-4-yl}-[4- | | | |
| | (5-trifluoromethyl-benzotriazol-1-yl)- | | | |
| | piperidin-1-yl]-methanone | | | |
| 245 | 4-(4-{4-[4-(5-Trifluoromethyl-benzotriazol- | 580 | 581 [M + H]+ | ++ |
| | 1-yl)-piperidine-1-carbonyl]-thiazol-2-yl}- | | | |
| | piperazin-1-yl-methyl)-benzonitrile | | | |
| 246 | (2-Morpholin-4-yl-oxazol-4-yl)-[4-(3- | 410 | 411 [M + H]+ | + |
| | trifluoromethyl-phenyl)-piperazin-1-yl]- | | | |
| | methanone | | | |

¹⁾The biological data refer to results obtained from the NF-κB inflammation assay.
[“+” stands for 50-80% inhibition, “++” means 80-90% and “+++” stands for 90-100% inhibition]

Exemplary compounds of formula (III) of the present invention include, but are not limited to, the followings:

| | | | LC/(+)-ESI- | Biological |
|---|---|---|---|---|
| Cp. | Name | Mass | MS: | activity¹⁾ |
| 1 | 2-Morpholin-4-yl-thiazole-4-carboxylic | 357 | 358 [M + H]⁺ | + |
| | acid (5,6-dimethyl-1H-benzoimidazol-2- | | | |
| | yl)-amide | | | |
| 2 | 2-[3-(2-Trifluoromethoxy-phenyl)-ureido]- | 462 | 463 [M + H]⁺ | + |
| | thiazole-4-carboxylic acid (1H- | | | |
| | benzoimidazol-2-yl)-amide | | | |
| 4 | 2-(2-Fluoro-benzoylamino)-thiazole-4- | 409 | 410 [M + H]⁺ | + |
| | carboxylic acid (5,6-dimethyl-1H- | | | |
| | benzoimidazol-2-yl)-amide | | | |
| 6 | N-{4-[N′-(1H-Benzoimidazol-2-yl)- | 441 | 442 [M + H]⁺ | ++ |
| | guanidinocarbonyl]-thiazol-2-yl}-3,4- | | | |
| | difluoro-benzamide | | | |
| 7 | N-{4-[N′-(1H-Benzoimidazol-2-yl)- | 423 | 424 [M + H]⁺ | ++ |
| | guanidinocarbonyl]-thiazol-2-yl}-4-fluoro- | | | |
| | benzamide | | | |
| 8 | N-{4-[N′-(1H-Benzoimidazol-2-yl)- | 473 | 474 [M + H]⁺ | +++ |
| | guanidinocarbonyl]-oxazol-2-yl}-2- | | | |
| | trifluoromethoxy-benzamide | | | |
| 9 | 2-(2-Trifluoromethoxy-benzoylamino)- | 453 | 454 [M + H]⁺ | + |
| | thiazole-4-carboxylic acid (4- | | | |
| | dimethylamino-[1,3,5]triazin-2-yl)-amide | | | |
| 10 | N-{4-[N′-(1H-Benzoimidazol-2-yl)- | 483 | 484 [M + H]⁺ | ++ |
| | guanidinocarbonyl]-thiazol-2-yl}-4-bromo- | | | |
| | benzamide | | | |
| 11 | N-{4-[N′-(1H-Benzoimidazol-2-yl)- | 435 | 436 [M + H]⁺ | ++ |
| | guanidinocarbonyl]-thiazol-2-yl}-2- | | | |
| | methoxy-benzamide | | | |
| 12 | N-{4-[N′-(1H-Benzoimidazol-2-yl)- | 489 | 490 [M + H]⁺ | +++ |
| | guanidinocarbonyl]-thiazol-2-yl}-2- | | | |
| | trifluoromethoxy-benzamide | | | |
| 13 | N-(1H-Benzoimidazol-2-yl)-N′-{2-[4-(2- | 558 | 559 [M + H]⁺ | + |
| | trifluoromethoxy-benzoyl)-piperazin-1-yl]- | | | |
| | thiazole-4-carbonyl}-guanidine | | | |
| 14 | N-(1H-Benzoimidazol-2-yl)-N′-[2-(2,3- | 420 | 421 [M + H]⁺ | + |
| | dihydro-benzo[1,4]dioxin-2-yl)-thiazole-4- | | | |
| | carbonyl]-guanidine | | | |
| 25 | 2-(2-Trifluoromethoxy-benzoylamino)- | 475 | 476 [M + H]⁺ | +++ |
| | thiazole-4-carboxylic acid (5,6-dimethyl- | | | |
| | 1H-benzoimidazol-2-yl)-amide | | | |
| 26 | 2-(3-Fluoro-4-trifluoromethyl- | 536 | 537 [M + H]⁺ | +++ |
| | benzoylamino)-oxazole-4-carboxylic acid | | | |
| | ethyl ester | | | |
| 27 | N-{4-[N′-(1H-Benzoimidazol-2-yl)- | 475 | 476 [M + H]⁺ | +++ |
| | guanidinocarbonyl]-oxazol-2-yl}-3-fluoro- | | | |
| | 4-trifluoromethyl-benzamide | | | |
| 30 | N-(1H-Benzoimidazol-2-yl)-N′-[2-(4- | 460 | 461 [M + H]⁺ | +++ |
| | benzyl-piperazin-1-yl)-thiazole-4- | | | |
| | carbonyl]-guanidine | | | |
| 31 | 2-(2-Trifluoromethoxy-benzoylamino)- | 447 | 448 [M + H]⁺ | +++ |
| | thiazole-4-carboxylic acid (1H- | | | |
| | benzoimidazol-2-yl)-amide | | | |
| 36 | N-(1H-Benzoimidazol-2-yl)-N′-{2-[1-(2- | 557 | 558 [M + H]⁺ | +++ |
| | trifluoromethoxy-benzoyl)-piperidin-4-yl]- | | | |
| | thiazole-4-carbonyl}-guanidine | | | |
| 40 | 2-(2-Trifluoromethoxy-benzoylamino)- | 459 | 460 [M + H]⁺ | +++ |
| | oxazole-4-carboxylic acid (5,6-dimethyl- | | | |
| | 1H-benzoimidazol-2-yl)-amide | | | |
| 42 | 2-(2-Trifluoromethoxy-benzoylamino)- | 562 | 563 [M + H]⁺ | +++ |
| | thiazole-4-carboxylic acid [6-(4-methyl- | | | |
| | piperazin-1-yl)-benzothiazol-2-yl]-amide | | | |
| 43 | 2-(2-Trifluoromethoxy-benzoylamino)- | 492 | 493 [M + H]⁺ | +++ |
| | thiazole-4-carboxylic acid (5-nitro-1H- | | | |
| | benzoimidazol-2-yl)-amide | | | |
| 45 | N-{4-[N′-(1H-Benzoimidazol-2-yl)- | 487 | 488 [M + H]⁺ | +++ |
| | guanidinocarbonyl]-thiazol-2-yl}-2- | | | |
| | cyclohexyl-benzamide | | | |
| 46 | 2-(2-Trifluoromethoxy-benzoylamino)- | 461 | 462 [M + H]⁺ | +++ |
| | thiazole-4-carboxylic acid (1-methyl-1H- | | | |
| | benzoimidazol-2-yl)-amide | | | |
| 47 | 2-(2-Trifluoromethoxy-benzoylamino)- | 553 | 554 [M + H]⁺ | +++ |
| | thiazole-4-carboxylic acid [5-(propane-1- | | | |
| | sulfonyl)-1H-benzoimidazol-2-yl]-amide | | | |
| 49 | N-(1H-Benzoimidazol-2-yl)-N′-(2- | 371 | 372 [M + H]⁺ | +++ |
| | morpholin-4-yl-thiazole-4-carbonyl)- | | | |
| | guanidine | | | |
| 51 | N-{4-[N′-(1H-Benzoimidazol-2-yl)- | 473 | 474 [M + H]⁺ | +++ |
| | guanidinocarbonyl]-thiazol-2-yl}-4- | | | |
| | trifluoromethyl-benzamide | | | |
| 52 | 2-[1-(2-Trifluoromethoxy-benzoyl)- | 621 | 622 [M + H]⁺ | +++ |
| | piperidin-4-yl]-thiazole-4-carboxylic acid | | | |
| | [5-(propane-1-sulfonyl)-1H-benzoimidazol- | | | |
| | 2-yl]-amide | | | |
| 53 | N-{4-[N′-(1H-Benzoimidazol-2-yl)- | 491 | 492 [M + H]⁺ | +++ |
| | guanidinocarbonyl]-thiazol-2-yl}-2-fluoro- | | | |
| | 4-trifluoromethyl-benzamide | | | |
| 54 | 1-{4-[N′-(1H-Benzoimidazol-2-yl)- | 504 | 505 [M + H]⁺ | +++ |
| | guanidinocarbonyl]-thiazol-2-yl}-3-(2- | | | |
| | trifluoromethoxy-phenyl)-urea | | | |
| 56 | N-{4-[N′-(1H-Benzoimidazol-2-yl)- | 491 | 492 [M + H]⁺ | +++ |
| | guanidinocarbonyl]-thiazol-2-yl}-3-fluoro- | | | |
| | 4-trifluoromethyl-benzamide | | | |
| 57 | N-{4-[N′-(1H-Benzoimidazol-2-yl)- | 441 | 442 [M + H]⁺ | +++ |
| | guanidinocarbonyl]-thiazol-2-yl}-2,6- | | | |
| | difluoro-benzamide | | | |
| 91 | N-(1H-Benzoimidazol-2-yl)-N′-(2- | 371 | 372 [M + H]⁺ | + |
| | morpholin-4-yl-thiazole-4-carbonyl)- | | | |
| | guanidine | | | |
| 250 | N-(1H-Benzoimidazol-2-yl)-N′-{2-[1-(4- | 484 | 485 [M + H]+ | ++ |
| | cyano-benzyl)-piperidin-4-yl]-thiazole-4- | | | |
| | carbonyl}-guanidine | | | |
| 251 | N-Benzothiazol-2-yl-N′-{2-[1-(4-cyano- | 501 | 502 [M + H]+ | ++ |
| | benzyl)-piperidin-4-yl]-thiazole-4- | | | |
| | carbonyl}-guanidine | | | |
| 252 | N-(1H-Benzoimidazol-2-yl)-N′-{2-[4-(4- | 485 | 485 [M + H]+ | +++ |
| | cyano-benzyl)-piperazin-1-yl]-thiazole-4- | | | |
| | carbonyl}-guanidine | | | |
| 253 | N-{2-[4-(4-Cyano-benzyl)-piperazin-1-yl]- | 466 | 467 [M + H]+ | ++ |
| | thiazole-4-carbonyl}-N′-(4-methyl-thiazol- | | | |
| | 2-yl)-guanidine | | | |
| 254 | N-(4-Methyl-thiazol-2-yl)-N′-(2-morpholin- | 352 | 353 [M + H]+ | + |
| | 4-yl-thiazole-4-carbonyl)-guanidine | | | |
| 255 | N-(1H-Benzoimidazol-2-yl)-N′-(2- | 371 | 372 [M + H]+ | +++ |
| | morpholin-4-yl-thiazole-4-carbonyl)- | | | |
| | guanidine | | | |
| 256 | N-Benzothiazol-2-yl-N′-(2-morpholin-4-yl- | 388 | 389 [M + H]+ | +++ |
| | thiazole-4-carbonyl)-guanidine | | | |
| 257 | N-(1H-Benzoimidazol-2-yl)-N′-(2- | 355 | 356 [M + H]+ | ++ |
| | morpholin-4-yl-oxazole-4-carbonyl)- | | | |
| | guanidine | | | |
| 258 | N-Benzooxazol-2-yl-N′-(2-morpholin-4-yl- | 372 | 373 [M + H]+ | ++ |
| | thiazole-4-carbonyl)-guanidine | | | |
| 259 | N-(2-Morpholin-4-yl-thiazole-4-carbonyl)- | 417 | 418 [M + H]+ | ++ |
| | N′-(5-nitro-benzooxazol-2-yl)-guanidine | | | |
| 260 | N-(5-Methyl-benzooxazol-2-yl)-N′-(2- | 386 | 387 [M + H]+ | + |
| | morpholin-4-yl-thiazole-4-carbonyl)- | | | |
| | guanidine | | | |
| 261 | N-(5-Chloro-benzooxazol-2-yl)-N′-(2- | 406 | 407 [M + H]+ | ++ |
| | morpholin-4-yl-thiazole-4-carbonyl)- | | | |
| | guanidine | | | |
| 262 | N-{2-[1-(4-Cyano-benzyl)-piperidin-4-yl]- | 465 | 466 [M + H]+ | + |
| | thiazole-4-carbonyl}-N′-(4-methyl-thiazol- | | | |
| | 2-yl)-guanidine | | | |
| 263 | N-(6-Chloro-1H-benzoimidazol-2-yl)-N′- | 591 | 592 [M + H]+ | +++ |
| | {2-[1-(2-trifluoromethoxy-benzoyl)- | | | |
| | piperidin-4-yl]-thiazole-4-carbonyl}- | | | |
| | guanidine | | | |
| 264 | N-(5,6-Dichloro-1H-benzoimidazol-2-yl)- | 625 | 626 [M + H]+ | ++ |
| | N′-{2-[1-(2-trifluoromethoxy-benzoyl)- | | | |
| | piperidin-4-yl]-thiazole-4-carbonyl}- | | | |
| | guanidine | | | |
| 265 | 2-Morpholin-4-yl-thiazole-4-carboxylic | 490 | 491 [M + H]+ | |
| | acid [4-(5-trifluoromethyl-benzothiazol-2- | | | |
| | yl)-phenyl]-amide | | | |
| 266 | 2-Morpholin-4-yl-thiazole-4-carboxylic | 423 | 424 [M + H]+ | |
| | acid (5-benzothiazol-2-yl-pyridin-2-yl)- | | | |
| | amide | | | |
| 267 | 2-Morpholin-4-yl-thiazole-4-carboxylic | 440 | 441 [M + H]+ | |
| | acid (4-benzothiazol-2-yl-2-fluoro-phenyl)- | | | |
| | amide | | | |
| 268 | 2-Morpholin-4-yl-thiazole-4-carboxylic | 506 | 507 [M + H]+ | |
| | acid (4-benzothiazol-2-yl-2-trifluoromethoxy- | | | |
| | phenyl)-amide | | | |
| 269 | 2-(1-Furo[2,3-c]pyridin-4-yl-piperidin-4- | 548 | 549 [M + H]+ | |
| | yl)-thiazole-4-carboxylic acid (5-benzoyl- | | | |
| | 1H-benzoimidazol-2-yl)-amide | | | |
| 270 | 2-[1-(4,6-Dimethoxy-[1,3,5]triazin-2-yl)- | 570 | 571 [M + H]+ | |
| | piperidin-4-yl]-thiazole-4-carboxylic acid | | | |
| | (5-benzoyl-1H-benzoimidazol-2-yl)-amide | | | |
| 271 | 2-[1-(4,6-Dimethoxy-[1,3,5]triazin-2-yl)- | 501 | 502 [M + H]+ | |
| | piperidin-4-yl]-thiazole-4-carboxylic acid | | | |
| | (5-fluoro-benzothiazol-2-yl)-amide | | | |
| 272 | 2-[1-(4-Cyano-benzyl)-piperidin-4-yl]- | 546 | 547 [M + H⁺] | ++ |
| | thiazole-4-carboxylic acid (5-benzoyl-1H- | | | |
| | benzoimidazol-2-yl)-amide | | | |
| 273 | 2-(1-Thieno[3,2-d]pyrimidin-4-yl- | 565 | 566 [M + H⁺] | ++ |
| | piperidin-4-yl)-thiazole-4-carboxylic acid | | | |
| | (5-benzoyl-1H-benzoimidazol-2-yl)-amide | | | |
| 274 | 2-[1-(4,6-Dimethoxy-[1,3,5]triazin-2-yl)- | 570 | 571 [M + H⁺] | +++ |
| | piperidin-4-yl]-thiazole-4-carboxylic acid | | | |
| | (5-benzoyl-1H-benzoimidazol-2-yl)-amide | | | |
| 275 | 2-[1-(6-Trifluoromethyl-pyrimidin-4-yl)- | 577 | 578 [M + H⁺] | ++ |
| | piperidin-4-yl]-thiazole-4-carboxylic acid | | | |
| | (5-benzoyl-1H-benzoimidazol-2-yl)-amide | | | |
| 276 | 2-[1-(4-Cyano-benzyl)-piperidin-4-yl]- | 486 | 487 [M + H]+ | + |
| | thiazole-4-carboxylic acid (5-ethoxy-1H- | | | |
| | benzoimidazol-2-yl)-amide | | | |
| 277 | 2-[1-(4-Cyano-benzyl)-piperidin-4-yl]- | 476 | 477 [M + H]+ | ++ |
| | thiazole-4-carboxylic acid (5-chloro-1H- | | | |
| | benzoimidazol-2-yl)-amide | | | |
| 278 | 2-[1-(4-Cyano-benzyl)-piperidin-4-yl]- | 510 | 511 [M + H]+ | ++ |
| | thiazole-4-carboxylic acid (5,6-dichloro- | | | |
| | 1H-benzoimidazol-2-yl)-amide | | | |

¹⁾The biological data refer to results obtained from the NF-κB inflammation assay.
[“+” stands for 50-80% inhibition, “++” means 80-90% and “+++” stands for 90-100% inhibition]

Exemplary compounds of formula (Ih) of the present invention include, but are not limited to, the following:

| | | | LC/(+)-ESI- | Biological |
|---|---|---|---|---|
| Cp. | Name | Mass | MS: [M + H]⁺ | activity |
| 279 | 2-(1-(2,6-dimethoxypyrimidin-4- | 575 | 576 | + |
| | yl)piperidin-4-yl)-N-(6-(thiophene-3- | | | |
| | carbonyl)-1H-benzo[d]imidazol-2- | | | |
| | yl)thiazole-4-carboxamide | | | |
| 280 | 2-(1-(thieno[3,2-d]pyrimidin-4-yl)piperidin- | 571 | 572 | +++ |
| | 4-yl)-N-(6-(thiophene-3-carbonyl)-1H- | | | |
| | benzo[d]imidazol-2-yl)thiazole-4- | | | |
| | carboxamide | | | |
| 281 | 2-(1-(4,6-dimethoxy-1,3,5-triazin-2- | 576 | 577 | +++ |
| | yl)piperidin-4-yl)-N-(6-(thiophene-3- | | | |
| | carbonyl)-1H-benzo[d]imidazol-2- | | | |
| | yl)thiazole-4-carboxamide | | | |
| 282 | N-(6-benzoyl-1H-benzo[d]imidazol-2-yl)-2- | 569 | 570 | +++ |
| | (1-(2,6-dimethoxypyrimidin-4-yl)piperidin- | | | |
| | 4-yl)thiazole-4-carboxamide | | | |
| 283 | N-(5-acetylbenzo[d]thiazol-2-yl)-2-(1- | 520 | 521 | +++ |
| | (thieno[3,2-d]pyrimidin-4-yl)piperidin-4- | | | |
| | yl)thiazole-4-carboxamide | | | |
| 284 | N-(5-(3-fluoro-4- | 630 | 631 | + |
| | methoxybenzoyl)benzo[d]thiazol-2-yl)-2-(1- | | | |
| | (thieno[3,2-d]pyrimidin-4-yl)piperidin-4- | | | |
| | yl)thiazole-4-carboxamide | | | |
| 285 | N-(5-(4-hydroxy-3- | 628 | 629 | + |
| | methoxybenzoyl)benzo[d]thiazol-2-yl)-2-(1- | | | |
| | (thieno[3,2-d]pyrimidin-4-yl)piperidin-4- | | | |
| | yl)thiazole-4-carboxamide | | | |
| 286 | N-(6-benzoyl-1H-benzo[d]imidazol-2-yl)-2- | 577 | 578 | ++ |
| | (1-(thiophen-2-ylsulfonyl)piperidin-4- | | | |
| | yl)thiazole-4-carboxamide | | | |
| 287 | N-(5-benzoyl-1H-benzo[d]imidazol-2-yl)-2- | 619 | 620 | +++ |
| | (1-(6,7-dimethoxyquinazolin-4-yl)piperidin- | | | |
| | 4-yl)thiazole-4-carboxamide | | | |
| 288 | N-(5-(3-methoxybenzoyl)benzo[d]thiazol-2- | 612 | 613 | + |
| | yl)-2-(1-(thieno[3,2-d]pyrimidin-4- | | | |
| | yl)piperidin-4-yl)thiazole-4-carboxamide | | | |
| 289 | 2-(1-(7H-purin-6-yl)piperidin-4-yl)-N-(6- | 549 | 550 | +++ |
| | benzoyl-1H-benzo[d]imidazol-2-yl)thiazole- | | | |
| | 4-carboxamide | | | |
| 290 | N-(6-benzoyl-1H-benzo[d]imidazol-2-yl)-2- | 619 | 620 | +++ |
| | (1-(2,6-dichloropyridin-4- | | | |
| | ylcarbamoyl)piperidin-4-yl)thiazole-4- | | | |
| | carboxamide | | | |
| 291 | N-(6-benzoyl-1H-benzo[d]imidazol-2-yl)-2- | 533 | 534 | + |
| | (1-(4-cyanopyridin-2-yl)piperidin-4- | | | |
| | yl)thiazole-4-carboxamide | | | |
| 292 | N-(5-benzoyl-1H-benzo[d]imidazol-2-yl)-2- | 570 | 571 | +++ |
| | (1-(4,6-dimethoxy-1,3,5-triazin-2- | | | |
| | yl)piperidin-4-yl)thiazole-4-carboxamide | | | |
| 293 | N-(6-benzoyl-1H-benzo[d]imidazol-2-yl)-2- | 565 | 566 | ++ |
| | (1-(thieno[3,2-d]pyrimidin-4-yl)piperidin-4- | | | |
| | yl)thiazole-4-carboxamide | | | |
| 294 | N-(6-benzoyl-1H-benzo[d]imidazol-2-yl)-2- | 577 | 578 | ++ |
| | (1-(6-(trifluoromethyl)pyrimidin-4- | | | |
| | yl)piperidin-4-yl)thiazole-4-carboxamide | | | |
| 295 | N-(5-benzoyl-1H-benzo[d]imidazol-2-yl)-2- | 546 | 547 | ++ |
| | (1-(4-cyanobenzyl)piperidin-4-yl)thiazole-4- | | | |
| | carboxamide | | | |
| 296 | 2-(1-([1,2,4]triazolo[1,5-a]pyrimidin-7- | 549 | 550 | +++ |
| | yl)piperidin-4-yl)-N-(5-benzoyl-1H- | | | |
| | benzo[d]imidazol-2-yl)thiazole-4- | | | |
| | carboxamide | | | |
| 297 | N-(5-benzoyl-1H-benzo[d]imidazol-2-yl)-2- | 562 | 563 | +++ |
| | (1-(2-methylpyrazolo[1,5-a]pyrimidin-7- | | | |
| | yl)piperidin-4-yl)thiazole-4-carboxamide | | | |
| 298 | 4-{4-[4-(5-Benzoyl-1H-benzoimidazol-2- | 620 | 621 | + |
| | ylcarbamoyl)-thiazol-2-yl]-piperidin-1-yl}-5- | | | |
| | methyl-pyrrolo[2,1-f][1,2,4]triazine-6- | | | |
| | carboxylic acid methyl ester | | | |
| 299 | methyl 7-(4-(4-(5-benzoyl-1H- | 607 | 608 | ++ |
| | benzo[d]imidazol-2-ylcarbamoyl)thiazol-2- | | | |
| | yl)piperidin-1-yl)-[1,2,4]triazolo[1,5- | | | |
| | a]pyrimidine-2-carboxylate | | | |

¹⁾The biological data refer to results obtained from the NF-κB inflammation assay.
[“+” stands for an EC50 of 30-100 μM, “++” stands for an EC50 of 10-30 μM and “+++” stands for an EC50 of smaller than 10 μM.]

Exemplary compounds of formula (IIIa) of the present invention include, but are not limited to, the following:

| | | | LC/(+/−)-ESI- | |
|---|---|---|---|---|
| | | | MS: [M + H]⁺ | Biological |
| Cp. | Name | Mass | ([M − H]⁻) | activity¹⁾ |
| 300 | 2-(isonicotinamido)-N-(6- | 459 | 460 | + |
| | (methylsulfonyl)benzo[d]thiazol-2- | | | |
| | yl)thiazole-4-carboxamide | | | |
| 301 | 2-cinnamamido-N-(6- | 484 | 485 | + |
| | (methylsulfonyl)benzo[d]thiazol-2- | | | |
| | yl)thiazole-4-carboxamide | | | |
| 302 | 2-(furan-2-carboxamido)-N-(6- | 448 | 449 | + |
| | (methylsulfonyl)benzo[d]thiazol-2- | | | |
| | yl)thiazole-4-carboxamide | | | |
| 303 | N-(5,6-dimethyl-1H-benzo[d]imidazol-2-yl)- | 381 | 382 | + |
| | 2-(furan-2-carboxamido)thiazole-4- | | | |
| | carboxamide | | | |
| 304 | N-(5,6-dimethyl-1H-benzo[d]imidazol-2-yl)- | 392 | 393 | + |
| | 2-(isonicotinamido)thiazole-4-carboxamide | | | |
| 305 | 2-cinnamamido-N-(5,6-dimethyl-1H- | 417 | 418 | + |
| | benzo[d]imidazol-2-yl)thiazole-4- | | | |
| | carboxamide | | | |
| 306 | ethyl 2-(2-(2- | 519 | 518 [M − H]⁻ | +++ |
| | (trifluoromethoxy)benzamido)thiazole-4- | | | |
| | carboxamido)-1H-benzo[d]imidazole-6- | | | |
| | carboxylate | | | |
| 307 | ethyl 2-(2-(2- | 536 | 535 [M − H]⁻ | ++ |
| | (trifluoromethoxy)benzamido)thiazole-4- | | | |
| | carboxamido)benzo[d]thiazole-6-carboxylate | | | |
| 308 | 2-(2-chlorobenzamido)-N-(6-(phenylthio)- | 506 | 505 [M − H]⁻ | + |
| | 1H-benzo[d]imidazol-2-yl)thiazole-4- | | | |
| | carboxamide | | | |
| 309 | N-(5-benzoyl-1H-benzo[d]imidazol-2-yl)-2- | 501 | 500 [M − H]⁻ | ++ |
| | (2-chlorobenzamido)thiazole-4-carboxamide | | | |
| 310 | ethyl 2-(2-(2-chlorobenzamido)thiazole-4- | 486 | 485 [M − H]⁻ | + |
| | carboxamido)benzo[d]thiazole-6-carboxylate | | | |
| 311 | 2-(2-chlorobenzamido)-N-(6- | 482 | 481 [M − H]⁻ | ++ |
| | (trifluoromethyl)benzo[d]thiazol-2- | | | |
| | yl)thiazole-4-carboxamide | | | |
| 312 | 2-(2-chlorobenzamido)-N-(4- | 444 | 443 [M − H]⁻ | ++ |
| | methoxybenzo[d]thiazol-2-yl)thiazole-4- | | | |
| | carboxamide | | | |
| 313 | 2-(2-chlorobenzamido)-N-(6- | 428 | 427 [M − H]⁻ | +++ |
| | methylbenzo[d]thiazol-2-yl)thiazole-4- | | | |
| | carboxamide | | | |
| 314 | 2-(2-chlorobenzamido)-N-(6- | 459 | 458 [M − H]⁻ | ++ |
| | nitrobenzo[d]thiazol-2-yl)thiazole-4- | | | |
| | carboxamide | | | |
| 315 | 2-(2-chlorobenzamido)-N-(6- | 458 | 457 [M − H]⁻ | + |
| | ethoxybenzo[d]thiazol-2-yl)thiazole-4- | | | |
| | carboxamide | | | |
| 316 | 2-(2-chlorobenzamido)-N-(6- | 430 | 429 [M − H]⁻ | ++ |
| | hydroxybenzo[d]thiazol-2-yl)thiazole-4- | | | |
| | carboxamide | | | |
| 317 | 2-(2-chlorobenzamido)-N-(6- | 444 | 443 [M − H]⁻ | ++ |
| | methoxybenzo[d]thiazol-2-yl)thiazole-4- | | | |
| | carboxamide | | | |
| 318 | 2-(2-chlorobenzamido)-N-(4- | 428 | 427 [M − H]⁻ | + |
| | methylbenzo[d]thiazol-2-yl)thiazole-4- | | | |
| | carboxamide | | | |
| 319 | 2-(2-chlorobenzamido)-N-(6- | 432 | 431 [M − H]⁻ | +++ |
| | fluorobenzo[d]thiazol-2-yl)thiazole-4- | | | |
| | carboxamide | | | |
| 320 | 2-(2-chlorobenzamido)-N-(6- | 448 | 447 [M − H]⁻ | + |
| | chlorobenzo[d]thiazol-2-yl)thiazole-4- | | | |
| | carboxamide | | | |
| 321 | N-(6-bromobenzo[d]thiazol-2-yl)-2-(2- | 493 | 492 [M − H]⁻ | + |
| | chlorobenzamido)thiazole-4-carboxamide | | | |
| 322 | 2-(2-chlorobenzamido)-N-(4- | 448 | 447 [M − H]⁻ | +++ |
| | chlorobenzo[d]thiazol-2-yl)thiazole-4- | | | |
| | carboxamide | | | |
| 323 | 2-(2-chlorobenzamido)-N-(5,6- | 442 | 441 [M − H]⁻ | ++ |
| | dimethylbenzo[d]thiazol-2-yl)thiazole-4- | | | |
| | carboxamide | | | |
| 324 | 2-(2-chlorobenzamido)-N-(6- | 498 | 497 [M − H]⁻ | +++ |
| | (trifluoromethoxy)benzo[d]thiazol-2- | | | |
| | yl)thiazole-4-carboxamide | | | |
| 325 | 2-(2-chlorobenzamido)-N-(6- | 492 | 491 [M − H]⁻ | + |
| | (methylsulfonyl)benzo[d]thiazol-2- | | | |
| | yl)thiazole-4-carboxamide | | | |
| 326 | N-(5,6-dimethyl-1H-benzo[d]imidazol-2-yl)- | 479 | 478 [M − H]⁻ | + |
| | 2-(1-(thiophen-2- | | | |
| | yl)cyclohexanecarboxamido)thiazole-4- | | | |
| | carboxamide | | | |
| 327 | N-(5,6-dimethyl-1H-benzo[d]imidazol-2-yl)- | 431 | 430 [M − H]⁻ | + |
| | 2-(1- | | | |
| | phenylcyclopropanecarboxamido)thiazole-4- | | | |
| | carboxamide | | | |
| 328 | N-(5,6-dimethyl-1H-benzo[d]imidazol-2-yl)- | 425 | 424 [M − H]⁻ | + |
| | 2-(2,5-dimethylthiophene-3- | | | |
| | carboxamido)thiazole-4-carboxamide | | | |
| 329 | N-(5,6-dimethyl-1H-benzo[d]imidazol-2-yl)- | 385 | 386 | + |
| | 2-(tetrahydrofuran-3-carboxamido)thiazole- | | | |
| | 4-carboxamide | | | |
| 330 | N-(5,6-dimethyl-1H-benzo[d]imidazol-2-yl)- | 463 | 462 [M − H]⁻ | + |
| | 2-(5-methyl-2-(trifluoromethyl)furan-3- | | | |
| | carboxamido)thiazole-4-carboxamide | | | |
| 331 | N-(5,6-dimethyl-1H-benzo[d]imidazol-2-yl)- | 372 | 373 | + |
| | 2-(2-(dimethylamino)acetamido)thiazole-4- | | | |
| | carboxamide | | | |
| 332 | N-(5,6-dimethyl-1H-benzo[d]imidazol-2-yl)- | 480 | 479 [M − H]⁻ | + |
| | 2-(5-(morpholinomethyl)furan-2- | | | |
| | carboxamido)thiazole-4-carboxamide | | | |
| 333 | 2-(2-(2-methoxybenzamido)thiazole-4- | 454 | 453 [M − H]⁻ | +++ |
| | carboxamido)benzo[d]thiazole-6-carboxylic | | | |
| | acid | | | |
| 334 | 2-(2-methoxybenzamido)-N-(6-(phenylthio)- | 501 | 502 | ++ |
| | 1H-benzo[d]imidazol-2-yl)thiazole-4- | | | |
| | carboxamide | | | |
| 335 | N-(4-(5,6-dimethyl-1H-benzo[d]imidazol-2- | 449 | 448 [M − H]⁻ | +++ |
| | ylcarbamoyl)thiazol-2-yl)thieno[2,3- | | | |
| | b]pyrazine-6-carboxamide | | | |
| 336 | N-(5,6-dimethyl-1H-benzo[d]imidazol-2-yl)- | 477 | 476 [M − H]⁻ | + |
| | 2-(2-morpholinoisonicotinamido)thiazole-4- | | | |
| | carboxamide | | | |
| 337 | ethyl 2-(2-(2-methoxybenzamido)thiazole-4- | 482 | 481 [M − H]⁻ | + |
| | carboxamido)benzo[d]thiazole-6-carboxylate | | | |
| 338 | N-(5-benzoyl-1H-benzo[d]imidazol-2-yl)-2- | 496 | 495 [M − H]⁻ | +++ |
| | (2-methoxybenzamido)thiazole-4- | | | |
| | carboxamide | | | |
| 339 | 2-(2-methoxybenzamido)-N-(6- | 494 | 493 [M − H]⁻ | +++ |
| | (trifluoromethoxy)benzo[d]thiazol-2- | | | |
| | yl)thiazole-4-carboxamide | | | |
| 340 | 2-(2-methoxybenzamido)-N-(6- | 478 | 477 [M − H]⁻ | + |
| | (trifluoromethyl)benzo[d]thiazol-2- | | | |
| | yl)thiazole-4-carboxamide | | | |
| 341 | 2-(2-methoxybenzamido)-N-(4- | 440 | 441 | +++ |
| | methoxybenzo[d]thiazol-2-yl)thiazole-4- | | | |
| | carboxamide | | | |
| 342 | 2-(2-methoxybenzamido)-N-(6- | 424 | 423 [M − H]⁻ | + |
| | methylbenzo[d]thiazol-2-yl)thiazole-4- | | | |
| | carboxamide | | | |
| 343 | 2-(2-methoxybenzamido)-N-(6- | 455 | 454 [M − H]⁻ | + |
| | nitrobenzo[d]thiazol-2-yl)thiazole-4- | | | |
| | carboxamide | | | |
| 344 | N-(6-ethoxybenzo[d]thiazol-2-yl)-2-(2- | 454 | 453 [M − H]⁻ | + |
| | methoxybenzamido)thiazole-4-carboxamide | | | |
| 345 | tert-butyl 4-(5,6-dimethyl-1H- | 387 | 388 | + |
| | benzo[d]imidazol-2-ylcarbamoyl)thiazol-2- | | | |
| | ylcarbamate | | | |
| 346 | N-(6-methyl-1H-benzo[d]imidazol-2-yl)-2- | 461 | 462 | +++ |
| | (2-(trifluoromethoxy)benzamido)thiazole-4- | | | |
| | carboxamide | | | |
| 347 | N-(5-ethoxy-1H-benzo[d]imidazol-2-yl)-2- | 491 | 492 | +++ |
| | (2-(trifluoromethoxy)benzamido)thiazole-4- | | | |
| | carboxamide | | | |
| 348 | N-(6-fluoro-1H-benzo[d]imidazol-2-yl)-2-(2- | 465 | 466 | +++ |
| | (trifluoromethoxy)benzamido)thiazole-4- | | | |
| | carboxamide | | | |
| 349 | N-(1-ethyl-1H-benzo[d]imidazol-2-yl)-2-(2- | 475 | 476 | +++ |
| | (trifluoromethoxy)benzamido)thiazole-4- | | | |
| | carboxamide | | | |
| 350 | N-(5-nitro-1H-benzo[d]imidazol-2-yl)-2-(2- | 492 | 493 | +++ |
| | (trifluoromethoxy)benzamido)thiazole-4- | | | |
| | carboxamide | | | |
| 351 | N-(5-methyl-7-(trifluoromethyl)- | 531 | 532 | + |
| | [1,2,4]triazolo[1,5-a]pyrimidin-2-yl)-2-(2- | | | |
| | (trifluoromethoxy)benzamido)thiazole-4- | | | |
| | carboxamide | | | |
| 352 | N-(6-benzoyl-1H-benzo[d]imidazol-2-yl)-2- | 551 | 552 | + |
| | (2-(trifluoromethoxy)benzamido)thiazole-4- | | | |
| | carboxamide | | | |
| 353 | N-(1-methyl-1H-benzo[d]imidazol-2-yl)-2- | 461 | 462 | +++ |
| | (2-(trifluoromethoxy)benzamido)thiazole-4- | | | |
| | carboxamide | | | |
| 354 | N-(6-hydroxybenzo[d]thiazol-2-yl)-2-(2- | 426 | 425 [M − H]⁻ | +++ |
| | methoxybenzamido)thiazole-4-carboxamide | | | |
| 355 | 2-(2-methoxybenzamido)-N-(4- | 424 | 425 | +++ |
| | methylbenzo[d]thiazol-2-yl)thiazole-4- | | | |
| | carboxamide | | | |
| 356 | N-(6-fluorobenzo[d]thiazol-2-yl)-2-(2- | 428 | 429 | +++ |
| | methoxybenzamido)thiazole-4-carboxamide | | | |
| 357 | N-(6-bromobenzo[d]thiazol-2-yl)-2-(2- | 488 | 487 [M − H]⁻ | +++ |
| | methoxybenzamido)thiazole-4-carboxamide | | | |
| 358 | N-(6-chlorobenzo[d]thiazol-2-yl)-2-(2- | 444 | 445 | +++ |
| | methoxybenzamido)thiazole-4-carboxamide | | | |
| 359 | N-(4-chlorobenzo[d]thiazol-2-yl)-2-(2- | 444 | 445 | +++ |
| | methoxybenzamido)thiazole-4-carboxamide | | | |
| 360 | N-(5,6-dimethylbenzo[d]thiazol-2-yl)-2-(2- | 438 | 439 | +++ |
| | methoxybenzamido)thiazole-4-carboxamide | | | |
| 361 | 2-(2-methoxybenzamido)-N-(6- | 494 | 495 | +++ |
| | (trifluoromethoxy)benzo[d]thiazol-2- | | | |
| | yl)thiazole-4-carboxamide | | | |
| 362 | 2-(2-methoxybenzamido)-N-(6- | 488 | 489 | +++ |
| | (methylsulfonyl)benzo[d]thiazol-2- | | | |
| | yl)thiazole-4-carboxamide | | | |
| 363 | 2-{[2-(2-Trifluoromethoxy-benzoylamino)- | 536 | 537 | ++ |
| | thiazole-4-carbonyl]-amino}-benzothiazole- | | | |
| | 6-carboxylic acid ethyl ester | | | |
| 364 | N-(6-hydroxybenzo[d]thiazol-2-yl)-2-(2- | 480 | 481 | ++ |
| | (trifluoromethoxy)benzamido)thiazole-4- | | | |
| | carboxamide | | | |
| 365 | N-(6-chlorobenzo[d]thiazol-2-yl)-2-(2- | 498 | 499 | ++ |
| | (trifluoromethoxy)benzamido)thiazole-4- | | | |
| | carboxamide | | | |
| 366 | N-(6-fluorobenzo[d]thiazol-2-yl)-2-(2- | 482 | 483 | +++ |
| | (trifluoromethoxy)benzamido)thiazole-4- | | | |
| | carboxamide | | | |
| 367 | N-(6-bromobenzo[d]thiazol-2-yl)-2-(2- | 542 | 543 | +++ |
| | (trifluoromethoxy)benzamido)thiazole-4- | | | |
| | carboxamide | | | |
| 368 | N-(7-chlorobenzo[d]thiazol-2-yl)-2-(2- | 498 | 499 | + |
| | (trifluoromethoxy)benzamido)thiazole-4- | | | |
| | carboxamide | | | |
| 369 | N-(6-(methylsulfonyl)benzo[d]thiazol-2-yl)- | 542 | 541 [M − H]⁻ | ++ |
| | 2-(2-(trifluoromethoxy)benzamido)thiazole- | | | |
| | 4-carboxamide | | | |
| 370 | N-(6-nitrobenzo[d]thiazol-2-yl)-2-(2- | 509 | 510 | + |
| | (trifluoromethoxy)benzamido)thiazole-4- | | | |
| | carboxamide | | | |
| 371 | N-(5-nitrobenzo[d]thiazol-2-yl)-2-(2- | 509 | 510 | + |
| | (trifluoromethoxy)benzamido)thiazole-4- | | | |
| | carboxamide | | | |
| 372 | N-(5-methoxybenzo[d]thiazol-2-yl)-2-(2- | 494 | 495 | + |
| | (trifluoromethoxy)benzamido)thiazole-4- | | | |
| | carboxamide | | | |
| 373 | N-(7-methoxybenzo[d]thiazol-2-yl)-2-(2- | 494 | 495 | + |
| | (trifluoromethoxy)benzamido)thiazole-4- | | | |
| | carboxamide | | | |
| 374 | N-(5-ethoxybenzo[d]thiazol-2-yl)-2-(2- | 508 | 509 | + |
| | (trifluoromethoxy)benzamido)thiazole-4- | | | |
| | carboxamide | | | |
| 375 | 2-(2-(trifluoromethoxy)benzamido)-N-(5- | 532 | 533 | + |
| | (trifluoromethyl)benzo[d]thiazol-2- | | | |
| | yl)thiazole-4-carboxamide | | | |
| 376 | 2-(2-(trifluoromethoxy)benzamido)-N-(5- | 548 | 549 | + |
| | (trifluoromethoxy)benzo[d]thiazol-2- | | | |
| | yl)thiazole-4-carboxamide | | | |
| 377 | N-(5-fluorobenzo[d]thiazol-2-yl)-2-(2- | 482 | 481 [M − H]⁻ | + |
| | (trifluoromethoxy)benzamido)thiazole-4- | | | |
| | carboxamide | | | |

¹⁾The biological data refer to results obtained from the NF-κB inflammation assay.
[“+” stands for an EC50 of 30-100 μM, “++” stands for an EC50 of 10-30 μM and “+++” stands for an EC50 of smaller than 10 μM.]

Proteasome Assay:

The chymotryptic activity of the 20S proteasome (Immatics, Tübingen) was determined using a Tecan Ultra plate reader and Suc-LLVT-AMC as substrate (Bachem). In the wells of a black 96 well polypropylene plate, 2 μl of the respective inhibitor dissolved in DMSO were mixed with 50 μl substrate solution (25 mM HEPES pH 7.5 at 20° C., 0.5 mM EDTA and Suc-LLVT-AMC (in the appropriate concentration) and the reaction was initiated by adding 150 μl proteasome solution (1.3 μg/ml 20S proteasome in 25 mM HEPES pH 7.5 at 20° C., 0.5 mM EDTA, 0.033% (w/v) SDS). Substrate hydrolysis was followed by fluorescence spectroscopy (excitation wavelength: 360 nm; emission wavelength: 465 nm) for 20 min at 30° C. and initial velocities were calculated and expressed as change in relative fluorescence units (RFU) per second.

For the determination of the IC₅₀ values (concentration of inhibitor required for 50% inhibition) at least four different inhibitor concentrations were applied. Each data point was recorded in triplicates. Curves were fitted with the a suitable program.

T-Lymphocyte Proliferation Assay:

Inhibition of stimulated peripheral blood monocytes (PBMC).

PBMCs were isolated from the blood of healthy volunteers with the help of ACCUSPIN™ System Histopaque®-1077 tubes, washed and resuspended with 10⁶ cells/mi in Dulbecco's modified eagles medium, containing 10% fetal calf serum and 2 mM glutamine. The cells were stimulated with 2 μg/ml phytohemoagglutinin in the presence of test compound or blank vehicle for 72 h. 4 h prior to the end of the incubation period, 5-bromo-2′-desoxyuridine (BrdU) was added to label the proliferating cells. After the incubation, the cells were separated by centrifugation and the culture supernatant removed. Incorporated BrdU was quantified with the help of an enzyme-linked immunosorbent assay. For the determination of the IC₅₀ values (concentration of inhibitor required for 50% inhibition) at least four different inhibitor concentrations were applied. Each data point was recorded in triplicates. Curves were fitted with the a suitable program.

Based on results obtained in the T-lymphocyte proliferation assay, the compounds of the present invention are suitable for treating inflammatory diseases or diseases associated with T-cells.

Inhibition of NF-κB-Induced Inflammation:

For the determination of anti-inflammatory activity of the compounds the PRINCESS® NINA Instant Assay from Cell Culture Service GmBH was used. This assay is based on recombinant A549-NF-κB-SEAP reporter cells preseeded in 96-well flat bottom plates. As the transfected reportergen for SEAP (secreted embryonic alkaline phosphatase) is under transcriptional control of a NF-κB-responsive element, the expression of this reporter is activated upon stimulation with TNF-α. SEAP secretion into the culture supernatant can be detected by the chemiluminescent substrate CSPD®. Test compounds that inhibit the NF-κB activation show reduced SEAP activity and reduced luminescent readout.

Following 18 h of reactivation at 37° C., 5% CO₂ and 90% relative humidity, the cells were incubated with 0.01 up to 100 μM of test compound for 4.5 h before stimulation with 2 ng/ml TNF-α. After stimulation with TNF-α for 22 h endogenous phosphatases were inactivated and CSPD® substrate was supplied for 40 min. SEAP activity then was quantified by measuring luminescence as relative light units (RLU) using a Tecan Ultra reader. Each data point was recorded in quadruplicates and EC50 values were calculated via fitting function and the Microsoft Excel Solver.

Additional advantages, features and modifications will readily occur to those skilled in the art. Therefore, the invention in its broader aspects is not limited to the specific details, and representative devices, shown and described herein. Accordingly, various modifications may be made without departing from the spirit or scope of the general inventive concept as defined by the appended claims and their equivalents.

All documents referred to herein are specifically incorporated herein by reference in their entireties.

As used herein and in the following claims, articles such as “the”, “a” and “an” can connote the singular or plural.

In the present description and in the following claims, to the extent a numerical value is enumerated, such value is intended to refer to the exact value and values close to that value that would amount to an insubstantial change from the listed value.

1. A compound of formula (I) and/or a salt and/or a physiologically functional derivative thereof, 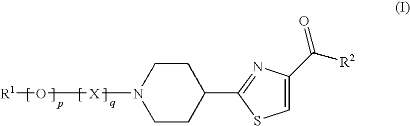
wherein
R¹ independently represents hydrogen, alkyl, cycloalkyl, hydroxyalkyl, haloalkyl, haloalkyloxy, aryl, substituted aryl, heteroaryl, substituted heteroaryl, arylalkyl or substituted arylalkyl;
R² independently represents —NR³R⁴, 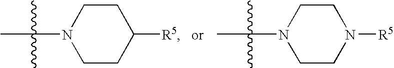
R³ independently represents alkyl, cycloalkyl, alkoxy, alkylamine, —OH, —SH, alkylthio, hydroxyalkyl, haloalkyl, haloalkyloxy, aryl or heteroaryl,
R⁴ independently represents alkyl, cycloalkyl, alkoxy, alkylamine, alkylthio, hydroxyalkyl, haloalkyl, haloalkyloxy, aryl or heteroaryl;
R⁵ independently represents H, COR⁶, CO₂R⁶, SOR⁶, SO₂R⁶, SO₃R⁶, alkyl, cycloalkyl, alkoxy, —NH₂, alkylamine, —NR⁷COR⁶, halogen, —OH, —SH, alkylthio, hydroxyalkyl, haloalkyl, haloalkyloxy, aryl or heteroaryl;
R⁶ independently represents H, alkyl, cycloalkyl, —NH₂, alkylamine, aryl or heteroaryl;
R⁷ independently represents H, alkyl, cycloalkyl, alkoxy, —OH, —SH, alkylthio, hydroxyalkyl, aryl, or heteroaryl;
p is 0, or 1;
q is 0, or 1;
X is CO or SO₂;
wherein an alkyl group, if not stated otherwise, denotes a linear or branched C₁-C₆-alkyl, preferably a linear or branched chain of one to five carbon atoms, a linear or branched C₂-C₆-alkenyl or a linear or branched C₂-C₆-alkinyl group, which can optionally be substituted by one or more substituents R′;
wherein R′ independently represents H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkyloxy, aryl, arylalkyl or heteroaryl;
wherein R″ independently represents H, haloalkyl, hydroxyalkyl, alkyl, cycloalkyl, aryl, heteroaryl or aminoalkyl;
wherein a cycloalkyl group denotes a non-aromatic ring system containing three to eight carbon atoms, wherein one or more of the carbon atoms in the ring can be substituted by a group E, E being O, S, SO, SO₂, N, or NR″, R″ being as defined above;
wherein an alkoxy group denotes an O-alkyl group, the alkyl group being as defined above; the alkoxy group is preferably a methoxy, ethoxy, isopropoxy, t-butoxy or pentoxy group;
wherein an alkylthio group denotes an S-alkyl group, the alkyl group being as defined above;
wherein an haloalkyl group denotes an alkyl group which is substituted by one to five halogen atoms, the alkyl group being as defined above;
wherein a hydroxyalkyl group denotes an HO-alkyl group, the alkyl group being as defined above;
wherein a haloalkyloxy group denotes an alkoxy group which is substituted by one to five halogen atoms, the alkyl group being as defined above;
wherein a hydroxyalkylamino group denotes an (HO-alkyl)₂-N— group or HO-alkyl-NH— group, the alkyl group being as defined above;
wherein an alkylamino group denotes an HN-alkyl or N-dialkyl group, the alkyl group being as defined above;
wherein a halogen group is chlorine, bromine, fluorine or iodine;
wherein an aryl group denotes an aromatic group having five to fifteen carbon atoms, which can optionally be substituted by one or more substituents R′, where R′ is as defined above;
wherein a heteroaryl group denotes a 5- or 6-membered heterocyclic group which contains at least one heteroatom like O, N, S, wherein said heterocyclic group can be fused to another ring and can optionally be substituted by one or more substituents R′, wherein R′ is as defined above.

2. A compound according to claim 1, wherein
p=0, q=1, and X═CO.

3. A compound according to claim 1, wherein
p=0, q=1, and X═SO₂.

4. A compound according to claim 1, wherein
p=1, q=1, and X═CO.

5. A compound according to claim 1, wherein
p=1, q=1, and X═SO₂.

6. A compound of formula (Ih) and/or a pharmaceutically acceptable salt thereof with an acid or a base, and or a pharmaceutically acceptable prodrug and/or a stereoisomer thereof, 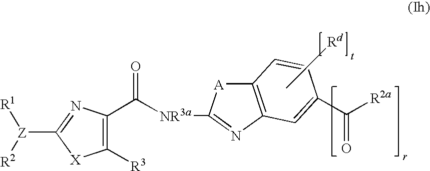
wherein
A is NR^{2′}, S or O;
t is 0 to 4;
r is 0, or 1;
R^{2a} is independently H, OH, SH, NH₂, alkyl, cycloalkyl, hydroxyalkyl, haloalkyl, haloalkyloxy, alkoxy, alkylamino, hydroxyalkylamino, halogen, aryl, or heteroaryl;
R^{3a} is H, OH, SH, NH₂, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, alkyl, cycloalkyl, hydroxyalkyl, haloalkyl, haloalkyloxy, alkoxy, alkylamino, hydroxyalkylamino, halogen, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R¹ is —C(O)R^{7a}, —C(O)CHR⁷R⁸, —C(O)NR⁷R⁸, —C(O)OR⁷, —R⁷C(O)R⁸, or —C(S)R^{7b};
R² is H, alkyl, cycloalkyl, heterocycloalkyl, haloalkyl, hydroxyalkyl, hydroxyalkylamino, alkylamino, or heteroaryl;
or R¹ and R² together with the N-atom or the C-atom to which they are attached form a 3 to 8 membered saturated or at least partially unsaturated monocycloc or polycyclic ring system, wherein at least one or more of the carbon atoms in the ring is a heteroatom selected from O, N, and S and the ring can be substituted by one or more R⁹;
R³ is H, —C(O)NR^{a}R^{b}, halogen, alkyl, haloalkyl, aryl, heteroaryl, OH, SH, NR^{4′}OR^{5′}, NH₂, hydroxyalkylamino, alkylamino, alkoxy, cycloalkyl, heterocycloalkyl, hydroxyalkyl, or haloalkyloxy;
R^{a} is H, halogen, alkyl, —C(NR⁷)NR^{7′}R⁸, —(CH₂)ₚaryl, —(CH₂)ₚNR⁷R⁸, aryl, —C(O)NR⁷R⁸, —N═CR⁷R⁸, —NR⁷C(O)R⁸, cycloalkyl, heterocycloalkyl, haloalkyl, hydroxyalkyl, hydroxyalkylamino, alkylamino, or heteroaryl;
R^{b} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, cycloalkyl, alkylamino, alkoxy, alkylthio, halogen, haloalkyl, haloalkyloxy, —O(CH₂)ₚ[O(CH₂)ₚ]_{q}OCH₃, —C(NR^{4″})NR^{4′}benzimidazolyl, —C(NR^{4″})NR^{4′}-benzthiazolyl, —C(NR^{4″})NR^{4′}benzoxazolyl, hydroxyalkyl, hydroxycycloalkyl, hydroxyalkylamino, heterocycloalkyl, aryl or heteroaryl;
R^{4′}, R^{4″}, R^{5′} independently are H, halogen, alkyl, —C(NR⁷)NR^{7′}R⁸, —(CH₂)ₚaryl, haloalkyl, —CH₂)ₚNR⁷R⁸, —C(O)NR⁷R⁸, —N═CR⁷R⁸, —NR⁷C(O)R⁸, cycloalkyl, heterocycloalkyl, hydroxyalkyl, hydroxyalkylamino, alkylamino, heteroaryl, or aryl;
R⁷, R^{7′}, R⁸ independently are H, halogen, alkyl, cycloalkyl, heterocycloalkyl, haloalkyl, hydroxyalkyl, hydroxyalkylamino, alkylamino, arylamino heteroaryl, or aryl;
R^{7a} is cycloalkyl, haloalkyl, hydroxyalkyl, hydroxyalkylamino, heteroaryl, or aryl;
R^{7b} is H, halogen, alkyl, cycloalkyl, heterocycloalkyl, haloalkyl, hydroxyalkyl, hydroxyalkylamino, heteroaryl, or aryl;
X is NR^{2′}, O, or S;
Z is N or CR^{2′};
R^{2′} is H, alkyl, —C(O)NR⁷, —C(O)R^{b}, cycloalkyl, heterocycloalkyl, haloalkyl, hydroxyalkyl, hydroxyalkylamino, alkylamino, heteroaryl, or aryl;
p is 1 to 6;
q is 1 to 6;
R⁹ independently represents H, —CN, —OH, —SH, alkoxy, alkylthio, —CO₂R^{4′}, —C(O)R^{4a}, —C(O)NR⁷R⁸, —SO₂NR^{4′}, —NR^{4′}R^{5′}, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, halogen, haloalkyl, alkylamino, —O(CH₂)ₚ[O(CH₂)ₚ]_{q}OCH₃, —C(NR^{4″})NR^{4′}benzimidazolyl, —C(NR^{4″})NR^{4′}-benzthiazolyl, —C(NR^{4″})NR^{4′}benzoxazolyl, hydroxycycloalkyl, hydroxyalkylamino, haloalkyloxy, heterocycloalkyl, —(CH₂)ₚNR⁷COR⁸, aryl, or heteroaryl;
R^{4a} is H, C₁-C₆-alkyl, C₂-C₆-alkenyl, cycloalkyl, haloalkyl, hydroxyalkyl, hydroxyalkylamino, alkylamino, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, halogen, heteroaryl, or aryl;
wherein
an C₁-C₆-alkyl group, if not stated otherwise, denotes a linear or branched C₁-C₆-alkyl, which can optionally be substituted by one or more substituents R′;
R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;
R″ is independently H, haloalkyl, hydroxyalkyl, alkyl, cycloalkyl, aryl, or heteroaryl;
an C₂-C₆-alkenyl group, if not stated otherwise, denotes a linear or branched C₂-C₆-alkenyl, which can optionally be substituted by one or more substituents R′;
an alkyl group, if not stated otherwise, denotes a linear or branched C₁-C₆-alkyl, a linear or branched C₂-C₆-alkenyl or a linear or branched C₂-C₆-alkynyl group, which can be substituted by one or more substituents R′; R′ being defined as above.
a cycloalkyl group denotes a non-aromatic ring system containing three to eight carbon atoms, wherein one or more of the carbon atoms in the ring can be substituted by one or more substituents R′; R′ being defined as above;
a heterocycloalkyl group denotes a non-aromatic ring system containing two to ten carbon atoms and at least one heteroatom selected from O, N, and S, wherein one or more of the carbon atoms in the ring can be substituted by R′ being as defined above;
an alkoxy group denotes an O-alkyl group, the alkyl group being as defined above;
an alkylthio group denotes an S-alkyl group, the alkyl group being as defined above;
an haloalkyl group denotes an alkyl group which is substituted by one to five halogen atoms, the alkyl group being as defined above;
a hydroxyalkyl group denotes an HO-alkyl group, the alkyl group being as defined above;
an haloalkyloxy group denotes an alkoxy group which is substituted by one to five halogen atoms, the alkyl group being as defined above;
a hydroxyalkylamino group denotes an (HO-alkyl)₂-N— group or HO-alkyl-NH— group, the alkyl group being as defined above;
an alkylamino group denotes an HN-alkyl or N-dialkyl group, the alkyl group being as defined above;
a halogen group is fluorine, chlorine, bromine, or iodine;
an aryl group denotes an aromatic group having five to fifteen carbon atoms, which can be substituted by one or more substituents R′, where R′ is as defined above;
an arylamino group denotes an HN-aryl or N-diaryl group, the aryl group being as defined above;
a heteroaryl group denotes a 5- to 10-membered aromatic heterocyclic group which contains at least one heteroatom selected from O, N, and S, wherein the heterocyclic group may be fused to another ring and the heterocyclic group or the fused ring can both be substituted independently by one or more substituents R′, wherein R′ is as defined above.

7. A compound of formula (Ia) and/or a pharmaceutically acceptable salt thereof with an acid or a base, and/or a pharmaceutically acceptable prodrug and/or a stereoisomer thereof, 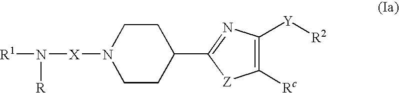
R is independently hydrogen, alkyl, cycloalkyl, hydroxyalkyl, haloalkyl, haloalkyloxy, aryl, or heteroaryl;
R¹ is independently alkyl, cycloalkyl, hydroxyalkyl, haloalkyl, haloalkyloxy, aryl, or heteroaryl;
X is CO, CS or SO₂;
Y is CO, CS or SO₂;
Z is NR^{2′}, S, or O;
R^{2′} is H, alkyl, —C(O)NR⁷, —C(O)R^{e}, cycloalkyl, haloalkyl, hydroxyalkyl, hydroxyalkylamino, alkylamino, heteroaryl, or aryl;
R^{c} is independently H, OH, SH, NROR¹, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R⁷, R^{7′} independently represent H, alkyl, cycloalkyl, haloalkyl, hydroxyalkyl, hydroxyalkylamino, alkylamino, heteroaryl or aryl;
R⁸ is H, NH₂, alkyl, cycloalkyl, haloalkyl, hydroxyalkyl, hydroxyalkylamino, alkylamino, heteroaryl or aryl;
R^{e} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, aryl hydroxyalkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4″-}benzthiazolyl, —C(NR^{4″})NR^{4′}benz-oxazolyl, or heteroaryl;
R^{4′}, R^{4″}, R^{5′} independently represent H, alkyl, cycloalkyl, haloalkyl, hydroxyalkyl, hydroxyalkylamino, alkylamino, —C(NR⁷)NR^{7′}R⁸, —(CH₂)ₚaryl, —CH₂)ₚNR⁷R⁸, —C(O)NR⁷R⁸, —N═CR⁷R⁸, —NR⁷C(O)R⁸, halogen, heteroaryl, or aryl
p is 1 to 6;
q is 1 to 6;
R² is independently 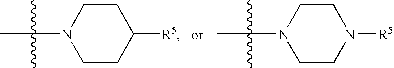
R⁵ is independently H, COR⁶, CO₂R⁶, SOR⁶, SO₂R⁶, SO₃R⁶, alkyl, cycloalkyl, alkoxy, —NH₂, alkylamine, —NR⁷COR⁶, halogen, —OH, —SH, alkylthio, hydroxyalkyl, haloalkyl, haloalkyloxy, aryl or heteroaryl;
R⁶ is independently H, alkyl, cycloalkyl, amino, haloalkyl, hydroxyalkyl, hydroxyalkylamino, alkylamino, aryl or heteroaryl;
wherein
an alkyl group, if not stated otherwise, denotes a linear or branched C₁-C₆-alkyl, a linear or branched C₂-C₆-alkenyl or a linear or branched C₂-C₆-alkynyl group, which can be substituted by one or more substituents R′, R′, being defined as above.
R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;
R″ is independently H, haloalkyl, hydroxyalkyl, alkyl, cycloalkyl, aryl, or heteroaryl;
a cycloalkyl group denotes a non-aromatic ring system containing three to eight carbon atoms, wherein one or more of the carbon atoms in the ring can be substituted by a group E, E being O, S, SO, SO₂, N, or NR″, R″ being as defined above;
an alkoxy group denotes an O-alkyl group, the alkyl group being as defined above;
an alkylthio group denotes an S-alkyl group, the alkyl group being as defined above;
a haloalkyl group denotes an alkyl group which is substituted by one to five halogen atoms, the alkyl group being as defined above;
a hydroxyalkyl group denotes an HO-alkyl group, the alkyl group being as defined above;
a haloalkyloxy group denotes an alkoxy group which is substituted by one to five halogen atoms, the alkyl group being as defined above;
a hydroxyalkylamino group denotes an (HO-alkyl)₂-N— group or HO-alkyl-NH— group, the alkyl group being as defined above;
an alkylamino group denotes an HN-alkyl or N-dialkyl group, the alkyl group being as defined above;
a halogen group is fluorine, chlorine, bromine, or iodine;
an aryl group denotes an aromatic group having five to fifteen carbon atoms, which can be substituted by one or more substituents R′, where R′ is as defined above;
a heteroaryl group denotes a 5- to 10-membered aromatic heterocyclic group which contains at least one heteroatom selected from O, N, and S, wherein the heterocyclic group may be fused to another ring and the heterocyclic group or the fused ring can both be substituted independently by one or more substituents R′, wherein R′ is as defined above.

8. A compound of formula (Ib) and/or a pharmaceutically acceptable salt thereof with an acid or a base, and/or a pharmaceutically acceptable prodrug and/or a stereoisomer thereof, 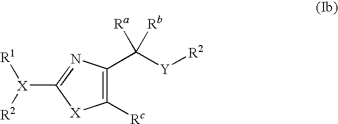
wherein
R¹ is —C(O)R⁷, —C(O)CHR⁷R⁸, —C(O)NR⁷R⁸, —C(O)OR⁷, —R⁷C(O)R⁸, or —C(S)R⁷;
R⁹ independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, heteroaryl, hydroxyalkyl, cycloalkyl, alkylamino, aryl hydroxyalkylamino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′-}benzthiazolyl, —C(NR^{4″})NR^{4′}benzoxazolyl, —(CH₂)ₚNR⁷COR⁸, or alkyl;
R⁴ is H, alkyl, cycloalkyl, haloalkyl, hydroxyalkyl, hydroxyalkylamino, alkylamino, heteroaryl, or aryl;
or R¹ and R⁴ together with the X to which they are attached form a 3 to 8 membered saturated or at least partially unsaturated monocyclic or polycyclic ring system, wherein at least one ring atom is a heteroatom selected from O, N, and S and the ring optionally has one or more substituents R⁹;
X is N, or CR^{2′};
Y is CO, CS or SO₂;
Z is NR^{2″}, S, or O;
R^{2″} is H, alkyl, —C(O)NR⁷, —C(O)R^{e}, cycloalkyl, haloalkyl, hydroxyalkyl, hydroxyalkylamino, alkylamino, heteroaryl, or aryl;
R^{2′} is H, alkyl, —C(O)NR^{4′}, —C(O)R^{4′}, cycloalkyl, haloalkyl, hydroxyalkyl, hydroxyalkylamino, alkylamino, heteroaryl, or aryl;
R^{4′}, R^{4″}, R^{5′} independently represent H, alkyl, cycloalkyl, haloalkyl, hydroxyalkyl, hydroxyalkylamino, alkylamino, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, halogen, heteroaryl, or aryl;
p is 1 to 6;
q is 1 to 6;
R^{a} is independently H, OH, SH, NH₂, alkyl, cycloalkyl, hydroxyalkyl, haloalkyl, haloalkyloxy, alkoxy, alkylamino, hydroxyalkylamino, halogen, aryl, or heteroaryl;
R^{b} is independently H, OH, SH, NH₂, alkyl, cycloalkyl, hydroxyalkyl, haloalkyl, haloalkyloxy, alkoxy, alkylamino, hydroxyalkylamino, halogen, aryl, or heteroaryl;
R^{c} is independently H, OH, SH, NR^{4′}OR^{5′}, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R⁷, R^{7′} independently represent H, alkyl, cycloalkyl, haloalkyl, hydroxyalkyl, hydroxyalkylamino, alkylamino, heteroaryl or aryl;
R⁸ is H, NH₂, alkyl, cycloalkyl, haloalkyl, hydroxyalkyl, hydroxyalkylamino, alkylamino, heteroaryl or aryl;
R^{e} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, hydroxy-alkylamino, alkoxy, alkylthio, —O(CH₂)ₚ[O(CH₂)ₚ]_{q}OCH₃, —C(NR^{4″})NR^{4′}benz-imidazolyl, —C(NR^{4″})NR^{4′}-benzthiazolyl, —C(NR^{4″})NR^{4′}benzoxazolyl, aryl or heteroaryl;
R² is independently 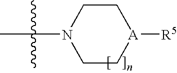
A is N, O, or CR^{2′};
R⁵ is independently H, SOR⁷, SO₂R⁷, SO₃R⁷, —C(O)R⁷, —C(O)CHR⁷R⁸, —C(O)NR⁷R⁸, —C(O)OR⁷, —R⁷C(O)R⁸, —C(S)R⁷, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R^{7′}, alkyl, cycloalkyl, alkoxy, —NH₂, alkylamino, hydroxyalkylamino, halogen, —OH, —SH, alkylthio, hydroxyalkyl, haloalkyl, haloalkyloxy, aryl or heteroaryl;
n is 0 to 2;
wherein
an alkyl group, if not stated otherwise, denotes a linear or branched C₁-C₆-alkyl, a linear or branched C₂-C₆-alkenyl or a linear or branched C₂-C₆-alkynyl group, which can be substituted by one or more substituents R′; R′ being defined as above;
R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;
R″ is independently H, haloalkyl, hydroxyalkyl, alkyl, cycloalkyl, aryl, or heteroaryl;
a cycloalkyl group denotes a non-aromatic ring system containing three to eight carbon atoms, wherein one or more of the carbon atoms in the ring can be substituted by a group E, E being O, S, SO, SO₂, N, or NR″, R″ being as defined above;
an alkoxy group denotes an O-alkyl group, the alkyl group being as defined above;
an alkylthio group denotes an S-alkyl group, the alkyl group being as defined above;
a haloalkyl group denotes an alkyl group which is substituted by one to five halogen atoms, the alkyl group being as defined above;
a hydroxyalkyl group denotes an HO-alkyl group, the alkyl group being as defined above;
a haloalkyloxy group denotes an alkoxy group which is substituted by one to five halogen atoms, the alkyl group being as defined above;
a hydroxyalkylamino group denotes an (HO-alkyl)₂-N— group or HO-alkyl-NH— group, the alkyl group being as defined above;
an alkylamino group denotes an HN-alkyl or N-dialkyl group, the alkyl group being as defined above;
a halogen group is fluorine, chlorine, bromine, or iodine;
an aryl group denotes an aromatic group having five to fifteen carbon atoms, which can be substituted by one or more substituents R′, where R′ is as defined above;
a heteroaryl group denotes a 5- to 10-membered aromatic heterocyclic group which contains at least one heteroatom selected from O, N, and S, wherein the heterocyclic group may be fused to another ring and the heterocyclic group or the fused ring can both be substituted independently by one or more substituents R′, wherein R′ is as defined above.

9. A compound of formula (Ic) and/or a pharmaceutically acceptable salt thereof with an acid or a base, and/or a pharmaceutically acceptable prodrug and/or a stereoisomer thereof; 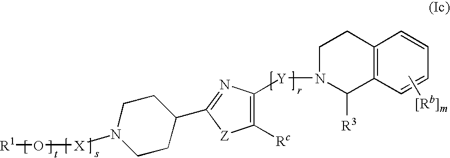
wherein
R¹ independently represents H, alkyl, cycloalkyl, hydroxyalkyl, haloalkyl, haloalkyloxy, aryl, or heteroaryl;
X is CO, CS or SO₂;
Y is CO, CS or SO₂;
Z is NR^{2″}, S, or O;
R^{2″} is H, alkyl, —C(O)NR⁷, —C(O)R^{e}, cycloalkyl, haloalkyl, hydroxyalkyl, hydroxyalkylamino, alkylamino, heteroaryl, or aryl;
R^{4′}, R^{4″}, R^{5′} independently represent H, alkyl, cycloalkyl, haloalkyl, hydroxyalkyl, hydroxyalkylamino, alkylamino, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, halogen, heteroaryl, or aryl
p is 1 to 6;
q is 1 to 6;
m is 0 to 4;
r is 0, or 1;
t is 0, or 1;
s is 0, or 1;
R^{b} is independently H, OH, SH, NR^{4′}OR^{5′}, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{c} is independently H, OH, SH, NR^{4′}OR^{5′}, NH₂, alkylamino, hydroxyalkylamino, halogen, CONR^{d}R^{e}, alkoxy, alkyl, cycloalkyl, hydroxyalkyl, haloalkyl, haloalkyloxy, aryl, or heteroaryl;
R^{d} is H, halogen, alkyl, —C(NR⁷)NR^{7′}R⁸, —(CH₂)ₚaryl, —(CH₂)ₚNR⁷R⁸, —C(O)NR⁷R⁸, —N═CR⁷R⁸, —NR⁷C(O)R⁸, cycloalkyl, haloalkyl, hydroxyalkyl, hydroxyalkylamino, alkylamino, heteroaryl or aryl;
R⁷, R^{7′} independently represent H, alkyl, cycloalkyl, haloalkyl, hydroxyalkyl, hydroxyalkylamino, alkylamino, heteroaryl or aryl;
R⁸ is H, NH₂, alkyl, cycloalkyl, haloalkyl, hydroxyalkyl, hydroxyalkylamino, alkylamino, heteroaryl or aryl;
R^{e} independently represents H, —CN, —OH, —SH, —CO₂R^{4′}, —C(O)R^{4′}, —SO₂NR^{4′}, —NR^{4′}R^{5′}, —C(O)NR⁷R⁸, —SO₂-alkyl, —SO₂R^{4′}, SO₃R^{4′}, —N═CR^{4′}R^{5′}, —NR^{4′}C(O)R^{4″}, —NR^{4′}—CO-haloalkyl, —NO₂, —NR^{4′}—SO₂-haloalkyl, —NR^{4′}—SO₂-alkyl, —NR^{4′}—CO-alkyl, —NR^{4′}(CH₂)ₚheteroaryl, alkyl, hydroxyalkyl, cycloalkyl, alkylamino, hydroxyalkyl-amino, alkoxy, alkylthio, —O(CH₂)ₚ[O(CH₂)ₚ]_{q}OCH₃, —C(NR^{4″})NR^{4′}benzimidazolyl, —C(NR^{4″})NR^{4′}-benzthiazolyl, —C(NR^{4″})NR^{4′}benz-oxazolyl, aryl or heteroaryl;
R³ is independently H, OH, SH, NR^{4′}OR^{5′}, NH₂, hydroxyalkylamino, alkylamino, halogen, CONR^{d}R^{e}, alkoxy, alkyl, cycloalkyl, hydroxyalkyl, haloalkyl, haloalkyloxy, aryl, or heteroaryl;
wherein
an alkyl group, if not stated otherwise, denotes a linear or branched C₁-C₆-alkyl, a linear or branched C₂-C₆-alkenyl or a linear or branched C₂-C₆-alkynyl group, which can be substituted by one or more substituents R′; R′ being defined as above;
R′ is independently H, —CO₂R″, —CONHR″, —CR″O, —SO₂NR″, —NR″—CO-haloalkyl, —NO₂, —NR″—SO₂-haloalkyl, —NR″—SO₂-alkyl, —SO₂-alkyl, —NR″—CO-alkyl, —CN, alkyl, cycloalkyl, alkylamino, alkoxy, —OH, —SH, alkylthio, hydroxyalkyl, hydroxyalkylamino, halogen, haloalkyl, haloalkyloxy, aryl, or heteroaryl;
R″ is independently H, haloalkyl, hydroxyalkyl, alkyl, cycloalkyl, aryl, or heteroaryl;
a cycloalkyl group denotes a non-aromatic ring system containing three to eight carbon atoms, wherein one or more of the carbon atoms in the ring can be substituted by a group E, E being O, S, SO, SO₂, N, or NR″, R″ being as defined above;
an alkoxy group denotes an O-alkyl group, the alkyl group being as defined above;
an alkylthio group denotes an S-alkyl group, the alkyl group being as defined above;
a haloalkyl group denotes an alkyl group which is substituted by one to five halogen atoms, the alkyl group being as defined above;
a hydroxyalkyl group denotes an HO-alkyl group, the alkyl group being as defined above;
a haloalkyloxy group denotes an alkoxy group which is substituted by one to five halogen atoms, the alkyl group being as defined above;
a hydroxyalkylamino group denotes an (HO-alkyl)₂-N— group or HO-alkyl-NH— group, the alkyl group being as defined above;
an alkylamino group denotes an HN-alkyl or N-dialkyl group, the alkyl group being as defined above;
a halogen group is fluorine, chlorine, bromine, or iodine;
an aryl group denotes an aromatic group having five to fifteen carbon atoms, which can be substituted by one or more substituents R′, where R′ is as defined above;
a heteroaryl group denotes a 5- to 10-membered aromatic heterocyclic group which contains at least one heteroatom selected from O, N, and S, wherein the heterocyclic group may be fused to another ring and the heterocyclic group or the fused ring can both be substituted independently by one or more substituents R′, wherein R′ is as defined above.

10. A composition comprising a compound according to claim 1 and a pharmaceutically acceptable carrier and/or diluent.

11. A method for the treatment and/or prevention of a disease characterized by hyperproliferation of cells, wherein said method comprises administering a compound according to claim 1 to a subject in need thereof.

12. A method for the treatment and/or prevention of a disease resulting from ischemia and/or reperfusion injury of organs and/or of parts of the body selected from the group consisting of heart, brain, peripheral limb, kidney, liver, spleen and lung, and/or wherein the endothelial dysfunction is associated with diseases selected from a group comprising infarctions such as myocardial infarction and critical limb ischemia, and/or wherein the endothelial dysfunction is associated with diseases selected from the group comprising ischemic diseases, myocardial infarction and ischemic diseases of organs, wherein said method comprises administering a compound according to claim 1 to a subject in need thereof.

13. A method for the treatment and/or prevention of a neurological disease and/or disorder selected from the group consisting of Alzheimer's disease, Parkinson's disease, Creutzfeld-Jacob Disease, Lewy Body Dementia, amyotrophic lateral sclerosis, stroke, epilepsy, multiple sclerosis, myasthenia gravis, Huntington's Disease, Down's Syndrome, nerve deafness, and Meniere's disease, wherein said method comprises administering a compound according to claim 1 to a subject in need thereof.

14. A method for the treatment and/or prevention of a disease that is caused by protozoal infestations in humans and animals, by bacteria, a virus and/or a proteinaceous agent, wherein said method comprises administering a compound according to claim 1 to a subject in need thereof.

15. A method for the treatment and/or prevention of a disease characterized by hyperproliferation of cells, wherein the disease is selected from the group consisting of psoriasis, atopic dermatitis, alopecia areata, alopecia totalis, alopecia subtotalis, alopecia universalis, alopecia diffusa, lupus erythematodes of the skin, lichen planus, dermatomyostis of the skin, atopic eczema, morphea, sklerodermia, psoriasis vulgaris, psoriasis capitis, psoriasis guttata, psoriasis inversa, alopecia areata ophiasis-type, androgenetic alopecia, allergic contact eczema, irritative contact eczema, contact eczema, pemphigus vulgaris, pemphigus foliaceus, pemphigus vegetans, scarring mucosal pemphigoid, bullous pemphgoid, mucous pemphigoid, dermatitis, dermatitis herpetiformis duhring, urticaria, necrobiosis lipoidica, erythema nodosum, lichen vidal, prurigo simplex, prurigo nodularis, prurigo acuta, linear IgA dermatosis, polymorphic light dermatoses, erythema solaris, lichen sclerosus et atrophicans, exanthema of the skin, drug exanthema, purpura chronica progressiva, dihidrotic ekzema, Ekzema, fixed drug exanthema, photoallergic skin reaction, lichen simplex eriorale, dermatitis and “Graft versus Host-Disease”, acne, rosacea, scarring, keloids, vitiligo, actinic keratoses, hyperkeratoses Hyperkeratosis Lenticularis Perstans, Keratosis pilaris and Ichthyoses, wherein said method comprises administering a compound according to claim 1 to a subject in need thereof.

16. A method for the treatment and/or prevention of a disease characterized by hyperproliferation of cells, wherein the disease is selected from the group consisting of hematological and solid tumors, wherein said method comprises administering a compound according to claim 1 to a subject in need thereof.

17. A method according to claim 16, wherein the disease is selected from the group consisting of prostate cancer, melanoma, ovarial cancer and multiple myeloma.

18. A method according to claim 16 for the treatment and/or prevention of an autoimmune disease and/or an inflammatory disease.

19. The method according to claim 18, wherein the disease is rheumatoid arthritis, multiple sclerosis, inflammatory bowel disease, inflammatory skin diseases and/or lupus erythematosus.

20. A method according to claim 16 for the treatment and/or prevention of stroke, reperfusion injury and/or Alzheimer's disease.

21. A method according to claim 16, for the treatment and/or prevention of a viral disease.

22. A method according to claim 21, wherein the viral disease is selected from the group consisting of hepatits B, hepatitis C, influenza virus infections, AIDS (HIV infections) and human papilloma virus infections.

23. A method according to claim 16, for the treatment and/or prevention of artheriosclerosis.

24. A method according to claim 16, for the treatment and/or prevention of osteoporosis.
